(19) United States
(12) Patent Application Publication
(10) Pub. No.: US 2026/0250595 A1
(43) Pub. Date: Aug. 27, 2026

(54) FLUORINE-CONTAINING ETHER COMPOUND, LUBRICANT FOR MAGNETIC RECORDING MEDIUM, AND MAGNETIC RECORDING MEDIUM

(71) Applicant: Resonac Corporation, Tokyo (JP)

(72) Inventors: Yutaka TANJI, Tokyo (JP); Daisuke YAGYU, Tokyo (JP); Kohei SAGA, Tokyo (JP)

(73) Assignee: Resonac Corporation, Tokyo (JP)

(21) Appl. No.: 18/861,646

(22) PCT Filed: Apr. 16, 2024
(86) PCT No.: PCT/JP2024/015067

§ 371 Date: Oct. 30, 2024

(30) Foreign Application Priority Data
Apr. 28, 2023 (JP) 2023-074981

(51) Int. Cl. C10M 147/04; C10N 40/18; G11B 5/725; G11B 5/84

(52) U.S. Cl. CPC C10M 147/04; G11B 5/7257; G11B 5/8408; C10M 2213/06; C10N 2040/18

(57) ABSTRACT

Provided is a fluorine-containing ether compound represented by Formula (1). R¹—CH₂—R²—CH₂—R³ (1) R² represents a perfluoropolyether chain. R¹ and R³ represent an end group having one to four polar groups and 1 to 50 carbon atoms. At least one of R¹ or R³ represents an end group represented by Formula (2). —O—X—CH(OH)—CH₂OH (2) X represents a divalent organic group having 2 to 30 carbon atoms, which may have at least one of one or two polar groups or one to three ether oxygen atoms. X has at least one carbon atom which is not bonded to any of the polar groups and the ether oxygen atoms.

### TECHNICAL FIELD

The present invention relates to a fluorine-containing ether compound, a lubricant for a magnetic recording medium, and a magnetic recording medium.

Priority is claimed on Japanese Patent Application No. 2023-074981, filed Apr. 28, 2023, the content of which is incorporated herein by reference.

### BACKGROUND ART

In order to improve the recording density of a magnetic recording and reproducing device, development of a magnetic recording medium suitable for a high recording density has been promoted. In the related art, as a magnetic recording medium, there is a magnetic recording medium in which a recording layer is formed on a substrate and a protective layer made of carbon or the like is formed on the recording layer. The protective layer protects information recorded on the recording layer and increases the slidability of a magnetic head. However, the durability of the magnetic recording medium is not sufficiently obtained only by providing the protective layer on the recording layer. Therefore, in general, a lubricating layer is formed by coating a surface of the protective layer with a lubricant.

As a lubricant used in a case of forming a lubricating layer of a magnetic recording medium, for example, a lubricant which contains a compound containing a polar group such as a hydroxy group or an amino group at an end of a fluorine-based polymer having a repeating structure containing —CF₂— has been proposed.

For example. Patent Document 1 and 2 disclose a fluorine-containing ether compound in which a glycerin linking group (—O—CH₂—CH(OH)—CH₂—O—) and an end group, which is an organic group having a polar group, are bonded to both ends of a perfluoropolyether chain in this order through a methylene group (—CH₂—).

Patent Document 3 discloses a fluorine-containing ether compound in which a linking group having a structure (—O—CH₂—CH(OH)—CH₂—(CH₂)ₙ—O—) in which a glycerin linking group is carbon-increased and an end group are bonded to both ends; of a perfluoropolyether chain in this order through a methylene group (—CH₂—).

Patent Document 4 discloses a fluorine-containing ether compound in which one or a plurality of glycerin structures are bonded to both ends of a perfluoropolyether chain through a methylene group (—CH₂—).

### CITATION LIST

### Patent Documents

Patent Document 1: PCT International Publication No. WO2021/090940
Patent Document 2: Japanese Patent Publication No. 6804893
Patent Document 3: PCT International Publication No. WO2019/054148
Patent Document 4: PCT International Publication No. WO2009/013785

### SUMMARY OF INVENTION

### Technical Problem

In recent years, in order to increase the capacity of a magnetic recording medium, further reduction in magnetic spacing (distance between a magnetic head and a magnetic layer of the magnetic recording medium) is required. Therefore, it is required to further reduce the thickness of the lubricating layer in the magnetic recording medium.

However, in general, in a case where the thickness of the lubricating layer is reduced, the corrosion resistance of the magnetic recording medium tends to be degraded. In addition, in a case where spin-off (a phenomenon in which a lubricant scatters or evaporates due to a centrifugal force and heat generation associated with the rotation of the magnetic recording medium) occurs in a case of a decrease in the thickness of the lubricating layer, a sufficient film thickness to satisfy the function of the lubricating layer may not be maintained. Therefore, there is a demand for a lubricating layer which has excellent corrosion resistance and can suppress spin-off even in a case where the thickness of the lubricating layer is reduced.

The present invention has been made in consideration of the above-described circumstances, and an object thereof is to provide a fluorine-containing ether compound which has excellent corrosion resistance, can form a lubricating layer capable of suppressing spin-off, and can be suitably used as a material of a lubricant for a magnetic recording medium.

Another object of the present invention is to provide a lubricant for a magnetic recording medium, which contains the fluorine-containing ether compound of the present invention and can form a lubricating layer having satisfactory corrosion resistance and capable of suppressing spin-off.

Still another object of the present invention is to provide a magnetic recording medium which has a lubricating layer containing the fluorine-containing ether compound of the present invention, has satisfactory corrosion resistance, and is capable of suppressing spin-off.

### Solution to Problem

The present invention includes the following aspects.
[1] A fluorine-containing ether compound represented by Formula (1).

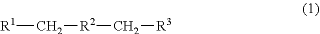

(In Formula (1). R² represents a perfluoropolyether chain. Each R¹ and R³ represents an end group having one to four polar groups and 1 to 50 carbon atoms. R¹ and R³ may be the same as or different from each other. At least one of R¹ or R³ represents an end group represented by Formula (2).)

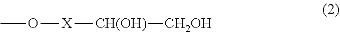

(In Formula (2), X represents a divalent organic group having 2 to 30 carbon atoms, which may have at least one of one or two polar groups or one to three ether oxygen atoms. X has at least one carbon atom which is bonded to neither the polar groups nor the ether oxygen atoms.)
[2] The fluorine-containing ether compound according to [1], in which the end group represented by Formula (2) is a group represented by any of Formulae (2-1) to (2-7).

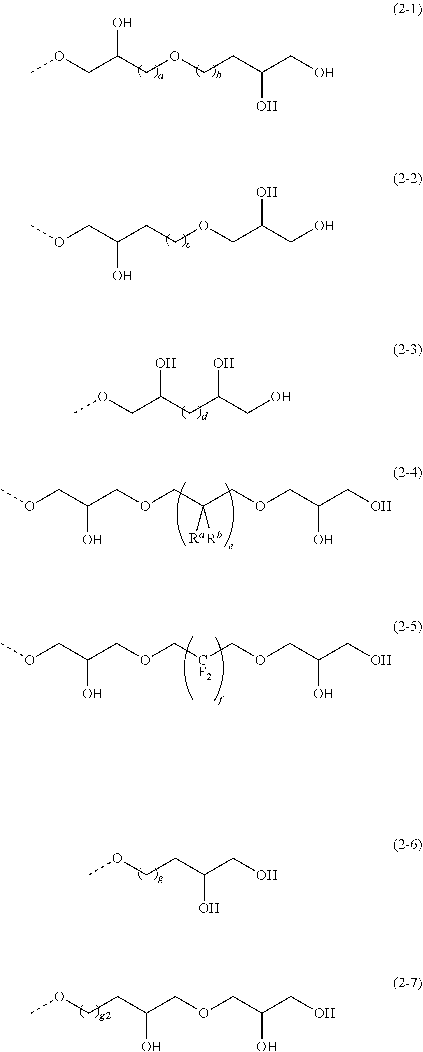

(In Formula (2-1), a represents an integer of 1 to 8, b represents an integer of 1 to 7.)

(In Formula (2-2), c represents an integer of 1 to 7.)

(In Formula (2-3), d represents an integer of 1 to 6.)

(In Formula (2-4), e represents an integer of 1 to 6, e pieces of R^{a}s and R^{b}s each independently represent a hydrogen atom or a methyl group.)

(In Formula (2-5), f represents an integer of 1 to 6.)

(In Formula (2-6), g represents an integer of 1 to 6.)

(In Formula (2-7), g2 represents an integer of 1 to 6.)

The fluorine-containing ether compound according to [1] or [2], in which R¹ and R³ in Formula (1) each independently represent an end group represented by Formula (2).
[4] The fluorine-containing ether compound according to any one of [1] to [3], in which R¹ and R³ in Formula (1) are the same as each other.
[5] The fluorine-containing ether compound according to [1] or [2], in which one of R¹ or R³ in Formula (1) represents an end group represented by Formula (2) and the other represents an end group represented by Formula (3) not corresponding to Formula (2).

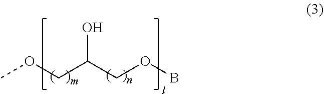

(In Formula (3), l represents an integer of 1 to 3. l pieces of m's each independently represent an integer of 1 to 6. l pieces of n's each independently represent an integer of 1 to 6. In one repeating unit, at least one of m or n represents 1. B represents an alkyl group which may have only one polar group, an organic group having one or more carbon-carbon unsaturated bonds, or a hydrogen atom.)
[6] The fluorine-containing ether compound according to any one of [1], [2], or [5], in which one of R¹ or R³ in Formula (1) represents an end group represented by Formula (2) and the other represents an end group represented by any of Formulae (3-1) to (3-3).

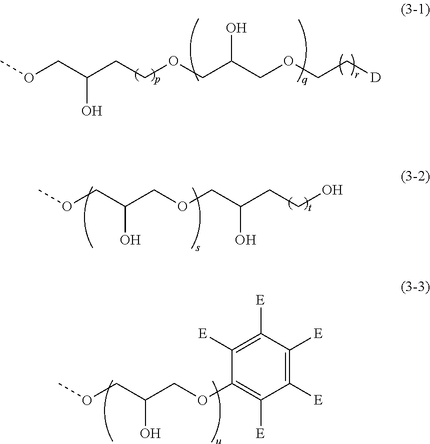

(In Formula (3-1), p represents an integer of 0 to 3, q represents an integer of 0 to 2. r represents an integer of 0 to 5. The total value of p and r is in a range of 1 to 5. D represents a polar group, a vinyl group, an ethynyl group, or an aryl group which may have a substituent.)

(In Formula (3-2), s represents an integer of 0 to 2. t represents an integer of 1 to 5.)

(In Formula (3-3), u represents an integer of 1 to 3. Five E's each independently represent a polar group. an alkoxy group having 1 to 8 carbon atoms, a halogeno group, or a hydrogen atom. Here, in a case where the five E's include a polar group, the number of polar groups in the five E's is 1.)
[7] The fluorine-containing ether compound according to any one of [1] to [6], in which a total number of polar groups included in R¹ and R³ in Formula (1) is in a range of 4 to 6.
[8] The fluorine-containing ether compound according to any one of [1] to [7], in which R² in Formula (1) represents a perfluoropolyether chain represented by Formula (4).

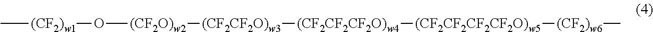

(In Formula (4), each w2, w3, w4, and w5 represents an average degree of polymerization, and each independently represent 0 to 20. Here, all of w2, w3, w4, and w5 do not represent 0 at the same time. Each w1 and w6 represents an average value representing the number of CFIs, and each independently represent 1 to 3. An arrangement order of (CF₂O), (CF₂CF₂O), (CF₂CF₂CF₂O), and (CF₂CF₂CF₂CF₂O), which are the repeating units in Formula (4), is not particularly limited.)
[9] The fluorine-containing ether compound according to any one of [1] to [8], in which R² in Formula (1) represents any one selected from perfluoropolyether chains represented by any of Formulae (4-1) to (4-4).

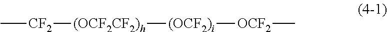

(In Formula (4-1), each h and i represents an average degree of polymerization, where h represents 1 to 20 and i represents 0 to 20.)

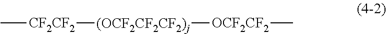

(In Formula (4-2), j represents an average degree of polymerization and represents 1 to 15.)

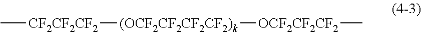

(In Formula (4-3), k represents an average degree of polymerization and represents 1 to 10.)

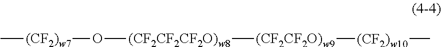

(In Formula (4-4), each w8 and w9 represents an average degree of polymerization, and each independently represents 1 to 20. Each w7 and w10 represents an average value representing the number of CF₂'s, and each independently represent 1 or 2.)
[10] The fluorine-containing ether compound according to any one of [1] to [9], in which the fluorine-containing ether compound has a number-average molecular weight of 500 to 10,000.
[11] A lubricant for a magnetic recording medium, including: the fluorine-containing ether compound according to any one of [1] to [10].
[12] A magnetic recording medium, which is provided with at least a magnetic layer, a protective layer, and a lubricating layer in this order on a substrate, in which the lubricating layer contains the fluorine-containing ether compound according to any one of [1] to [10].
[13] The magnetic recording medium according to [12], in which the lubricating layer has an average film thickness of 0.5 nm to 2.0 nm.

### Advantageous Effects of Invention

The fluorine-containing ether compound of the present invention is a compound represented by Formula (1), and is suitable as a material of a lubricant for a magnetic recording medium.

Since the lubricant for a magnetic recording medium of the present invention contains the fluorine-containing ether compound of the present invention, it is possible to form a lubricating layer that has satisfactory corrosion resistance and is capable of suppressing spin-off.

The magnetic recording medium of the present invention includes a lubricating layer containing the fluorine-containing ether compound of the present invention. Therefore, the magnetic recording medium of the present invention has satisfactory corrosion resistance, is capable of suppressing spin-off, and has excellent reliability and durability. Therefore, the magnetic recording medium of the present invention can reduce the thickness of the lubricating layer, and thus can contribute to the reduction of magnetic spacing and can further reduce the floating amount of the magnetic head.

### BRIEF DESCRIPTION OF DRAWINGS

FIG. 1 A schematic cross-sectional view showing an embodiment of a magnetic recording medium according to the present invention.

### DESCRIPTION OF EMBODIMENTS

In order to solve the above-described problems, the present inventors have conducted intensive examination as described below.

In the related art, as a material of a lubricant for a magnetic recording medium (hereinafter, also referred to as “lubricant”) which is applied to a surface of a protective layer, a fluorine-containing ether compound that contains an end group having a polar group such as a hydroxy group has been preferably used.

However, in a case where a lubricating layer having a small thickness is formed on a protective layer using the fluorine-containing ether compound that contains an end group having a polar group in the related art, it is difficult to realize a lubricating layer having satisfactory corrosion resistance and capable of suppressing spin-off as described below.

That is, the polar group in the fluorine-containing ether compound is bonded to the active point on the protective layer to improve the adhesion of the lubricating layer to the protective layer. In a case where the adhesion of the lubricating layer to the protective layer is insufficient, a state of coating the protective layer with the lubricating layer is insufficient, water, which causes corrosion, is likely to be taken into the protective layer, and thus sufficient corrosion resistance cannot be obtained.

In addition, the polar group in the fluorine-containing ether compound is involved not only in the interaction with the protective layer by being bonded to the active point on the protective layer, but also in the intramolecular or intermolecular interaction. In a case where the polar group involved in the intermolecular interaction between the fluorine-containing ether compounds is insufficient, the fluorine-containing ether compound in the lubricating layer is easily scattered with the rotation of the magnetic recording medium, which is likely to cause spin-off.

Therefore, the fluorine-containing ether compound is required to have a sufficient amount of the polar group involved in the interaction with the protective layer and the polar group involved in the intermolecular interaction so that a lubricating layer having satisfactory corrosion resistance and suppressed spin-off can be formed. However, in a case where the number of polar groups in the fluorine-containing ether compound is increased, the hydrophobicity of the lubricating layer containing the fluorine-containing ether compound is lowered, and thus sufficient corrosion resistance cannot be obtained.

Therefore, the present inventors have repeatedly conducted intensive examination by focusing on the strength of polarity of the polar group contained in the fluorine-containing ether compound and the interaction of the polar groups. As a result, it was considered that at least some of the polar groups in the fluorine-containing ether compound may be hydroxy groups having a 1,2-diol structure, which are disposed at the end of the perfluoropolyether chain. In this case, as described below, a hydroxy group capable of interacting with the protective layer and a hydroxy group capable of being involved in the intermolecular interaction can be ensured while a decrease in hydrophobicity due to the hydroxy group contained in the 1,2-diol structure is suppressed.

In the 1,2-diol structure —CH(OH)—CH₂OH), since carbon atoms, to which hydroxy groups are bonded, are bonded to each other, the distance between the hydroxy groups is short, and steric repulsion and electrostatic repulsion between the hydroxy groups are likely to occur. Therefore, two hydroxy groups contained in the 1,2-diol structure are disposed in opposite directions with respect to the carbon atom to which the 1,2-diol structure is bonded. Therefore, the dipole moments generated by two hydroxy groups contained in the 1,2-diol structure cancel each other, and an increase in polarity of the entire fluorine-containing ether compound molecules is suppressed.

In addition, the distance between the active points on the protective layer is sufficiently large as compared with the distance between the hydroxy groups contained in the 1,2-diol structure. Moreover, two hydroxy groups contained in the 1,2-diol structure are disposed in opposite directions with respect to the carbon atom to which the 1,2-diol structure is bonded. Therefore, two hydroxy groups contained in the 1,2-diol structure are not directed to be close to the protective layer at the same time, and only one hydroxy group between the two hydroxy groups can interact with the active point on the protective layer. Therefore, the other hydroxy group between the two hydroxy groups can be involved in the intermolecular interaction between the fluorine-containing ether compounds.

In the related art, the structure of the fluorine-containing ether compound used as a lubricant has been examined such that as many polar groups as possible in the compound are disposed to easily interacted with the active point on the protective layer in order to improve adhesion to the protective layer. Therefore, a structure in which adjacent hydroxy groups are directed in opposite directions on the protective layer tends to be avoided because the carbon atoms to which the hydroxy groups are bonded are bonded to each other.

However, the present inventors have ensured a polar group which can be involved in the intermolecular interaction between the fluorine-containing ether compounds by intentionally using some polar groups in the fluorine-containing ether compound as the hydroxy groups of the 1,2-diol structure. In addition, the present inventors have considered that the number of polar groups which can interact with the active point on the protective layer may be adjusted as necessary.

Further, the present inventors have repeatedly conducted examination on a fluorine-containing ether compound in which a 1,2-diol structure is disposed at one or both ends of a perfluoropolyether chain, in order to improve the corrosion resistance of a lubricating layer containing the compound and to suppress spin-off.

As a result, it has been found that a fluorine-containing ether compound in which an end group having one to four polar groups and 1 to 50 carbon atoms are disposed at each of both ends of the perfluoropolyether chain, and at least one of the two end groups is an end group having at least one carbon atom that is not bonded to any of the polar group and the ether oxygen atom, having a 1,2-diol structure, and represented by Formula (2) may be obtained, thereby completing the present invention.

In such a fluorine-containing ether compound, since the number of polar groups is appropriate and some of the polar groups are hydroxy groups having a 1,2-diol structure which are disposed at the end of the perfluoropolyether chain, hydroxy groups capable of interacting with the protective layer and hydroxy groups capable of being involved in the intermolecular interaction can be ensured while a decrease in hydrophobicity due to the polar groups is sufficiently suppressed. Further, the end group represented by Formula (2), which is disposed at least at one end, has at least one carbon atom that is not, bonded to any of the polar groups and the ether oxygen atoms. Since this carbon atom has an extremely low affinity for water, the interaction between the fluorine-containing ether compound molecule and water is strongly hindered. Therefore, even in a case where the fluorine-containing ether compound contains a plurality of polar groups, the hydrophobicity is sufficiently obtained. In addition, since the movement of the above-described carbon atom is suppressed, the carbon atom imparts moderate rigidity to the fluorine-containing ether compound molecule, and suppresses the intramolecular interaction between the polar groups, and thus the polar groups are likely to be involved in the intermolecular interaction between the fluorine-containing ether compounds.

As described above, the fluorine-containing ether compound can maintain low polarity while ensuring the number of polar groups involved in the interaction with the protective layer, and has sufficient hydrophobicity. Therefore, the lubricant containing this compound is unlikely to take in water, which causes corrosion, and thus the corrosion resistance is excellent. In addition, the above-described fluorine-containing ether compound easily generates a polar group which is not involved in the interaction with the protective layer and the intramolecular interaction, and the polar group easily forms an intermolecular interaction. Therefore, a lubricating layer in which scattering of the fluorine-containing ether compound is unlikely to occur with the rotation of the magnetic recording medium and which has excellent spin-off resistance can be formed.

On the contrary, for example, in a case where the fluorine-containing ether compound is extremely flexible, since the polar group contained in the fluorine-containing ether compound is likely to form an intramolecular interaction, the polar group is unlikely to be involved in an intermolecular interaction.

In addition, in a case where all of a plurality of polar groups in the fluorine-containing ether compound are disposed with a sufficient distance between adjacent polar groups, all the polar groups are likely to be involved in the interaction with the protective layer, and accordingly, the interaction between the fluorine-containing ether compounds due to the polar groups is not sufficiently obtained.

Further, the present inventors have confirmed that a lubricating layer having satisfactory corrosion resistance and capable of suppressing spin-off can be formed even in a case where the thickness thereof is reduced by using a lubricant containing the fluorine-containing ether compound described above, thereby completing the present invention.

Hereinafter, the fluorine-containing ether compound, the lubricant for a magnetic recording medium, and the magnetic recording medium of the present invention will be described in detail. Further, the present invention is not limited to the embodiments described below.

### (Fluorine-Containing Ether Compound)

The fluorine-containing ether compound of the present embodiment is represented by Formula (1).

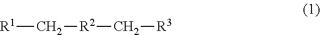

(In Formula (1), R² represents a perfluoropolyether chain. R and R³ represent an end group having one to four polar groups and 1 to 50 carbon atoms. R¹ and R³ may be the same as or different from each other. At least one of R¹ or R³ represents an end group represented by Formula (2.)

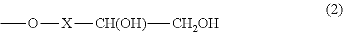

(In Formula (2). X represents a divalent organic group having 2 to 30 carbon atoms, which may have at least one of one or two polar groups or one to three ether oxygen atoms. X has at least one carbon atom which is bonded to neither the polar groups nor the ether oxygen atoms.)

As shown in Formula (1), the fluorine-containing ether compound of the present embodiment has an end group represented by R¹ which is bonded to one end of a skeleton of a perfluoropolyether chain (hereinafter, also referred to as a PIPE chain) represented by R² through a methylene group, and has an end group represented by R³ which is bonded to the other end of the skeleton through a methylene group.

(End Group Represented by R¹ and R³)

R¹ and R³ represent an end group having one to four polar groups and 1 to 50 carbon atoms. At least one of R¹ or R³ represents an end group represented by Formula (2).

In a case where one of R¹ or R³ represents an end group that does not correspond to Formula (2), the number of polar groups contained in the end group that does not correspond to Formula (2) in R¹ and R³ is 1 or more. In addition, the number of polar groups included in the end group represented by Formula (2) is 2 or more. Therefore, in a case where a lubricating layer is formed on the protective layer using a lubricant containing a fluorine-containing ether compound, a suitable interaction occurs between the lubricating layer and the protective layer. The number of polar groups included in R¹ and R³ is preferably 2 or more. In a case where the number of polar groups included in R¹ and R³ is 2 or more, the fluorine-containing ether compound can form a lubricating layer in which adhesion with the protective layer is excellent, an intermolecular interaction between the polar groups is suitably formed, and spin-off can be suppressed.

The numbers of polar groups included in R¹ and R³ are each 4 or less. Therefore, in the lubricating layer containing the fluorine-containing ether compound, the polarity of the fluorine-containing ether compound is not extremely high, and aggregation of the fluorine-containing ether compound and formation of a lump are suppressed so that the lubricating layer can be suppressed from losing the smoothness. The number of polar groups included in R^{f} and R is preferably 3 or less. In a case where the number of polar groups included in R¹ and R³ is 3 or less, the hydrophilicity of the fluorine-containing ether compound is not extremely high, and water, which causes corrosion, can be prevented from being taken into the magnetic recording medium so that a fluorine-containing ether compound that can form a lubricating layer with high corrosion resistance can be obtained.

The total number of polar groups included in R¹ and R³ in Formula (1) is preferably in a range of 3 to 6 and more preferably in a range of 4 to 6. In a case where the total number of the above-described polar groups is 3 or more, the interaction between the polar groups included in R¹ and R³ and the protective layer is effectively obtained. As a result, the fluorine-containing ether compound can form a lubricating layer having high adhesion to the protective layer. Therefore, a lubricating layer having more excellent spin-off resistance is obtained. In addition, in a case where the total number of the above-described polar groups is 6 or less, the polarity of the fluorine-containing ether compound is not extremely increased so that water, which causes corrosion, can be prevented from being taken into the layer. Therefore, a lubricating layer having more excellent corrosion resistance can be formed.

The polar groups included in R¹ and R³ may be partially or entirely the same as or different from each other. In addition, the number of polar groups included in R, and the number of polar groups included in R³ may be the same as or different from each other. The number of polar groups included in R¹ and the number of polar groups included in R³ are preferably the same as each other because the state of coating the protective layer with the fluorine-containing ether compound is more uniform, and a lubricating layer having satisfactory adhesion can be formed.

The polar group included in R¹ and R³ is preferably at least one polar group selected from the group consisting of a hydroxy group (—OH), a carboxy group (—COOH), a formyl group (—(C═O)H), a carbonyl group (—(C═O)R⁷; R⁷ represents an organic group), a sulfo group (—SO₃H), a cyano group (—CN), a group having an amide bond (—NR⁸COR⁹ or —CONR¹⁰R¹¹; R⁸, R⁹, R¹⁰, and R¹¹ each independently represent a hydrogen atom or an organic group), and an amino group (—NR¹²R¹³; R¹² and R¹³ each independently represent a hydrogen atom or an organic group). The group having an amide bond includes, as shown in the formula above, both a group (for example, a carboxamide group (—C(═O)NH₂)) bonded to a carbon atom constituting the amide bond and a group (for example, an acetamido group (—NHC(═O)CH₃)) bonded to a nitrogen atom constituting the amide bond. In the group having an amide bond, R⁸ and R⁹ may be bonded to each other to form a ring, and R¹⁰ and R¹¹ may be bonded to each other to form a ring. It is preferable that R⁸, R⁹, R¹⁰, and R¹¹ in the group having an amide bond are each independently selected from the group consisting of a hydrogen atom, a methyl group, an ethyl group, a propyl group, and a butyl group.

The term “polar group” in the present specification does not include a halogeno group (—F, —Cl, —Br, or the like) or an ether bond (—O—).

It is preferable that R¹ and R³ each independently include at least one polar group selected from the group consisting of a hydroxy group, a cyano group, and a group having an amide bond. The reason for this is that the hydroxy group, the cyano group, and the group having an amide bond are chemically stable, and a lubricating layer containing a fluorine-containing ether compound having these polar groups does not deteriorate for a long period of time. In addition, it is because the acidity of the hydroxy group, the cyano group, and the group having an amide bond is not extremely high, and thus the substrate is unlikely to be corroded.

It is preferable that the polar group included in R¹ and R³ in Formula (1) each include at least one hydroxy group. It is more preferable that the polar groups included in R¹ and R³ are all hydroxy groups. This is because, in a case where all the polar groups included in R¹ and R³ are hydroxy groups, the state of coating the protective layer with the fluorine-containing ether compound is more uniform.

The number of carbon atoms in the end group represented by R¹ and R³ is in a range of 1 to 50, preferably in a range of 3 to 20, and more preferably in a range of 4 to 15, in a case where the number of carbon atoms in the end group represented by R¹ and R³ is 1 or more, since the hydrophobicity of the end group can be ensured, water, which causes corrosion, can be prevented from being attracted to the lubricating layer, and thus a lubricating layer having satisfactory corrosion resistance is formed. In a case where the number of carbon atoms in the end group represented by R¹ and R³ is 50 or less, the end group has a flexible structure, and thus adhesion between the lubricating layer containing the fluorine-containing ether compound and the protective layer is improved. As a result, a lubricating layer capable of suppressing spin-off can be obtained.

At least one of R¹ or R³ represents an end group represented by Formula (2). The end group represented by Formula (2) has an oxygen atom (ether oxygen atom) bonded to a methylene group (—CH₂—) bonded to R². Oxygen atoms disposed at the ends of the end groups represented by Formula (2) form an ether bond (—O—) with atoms bonded to both sides of the oxygen atoms. This ether bond imparts moderate flexibility to the fluorine-containing ether compound represented by Formula (1), and increases the affinity between the polar group of the end group represented by Formula (2) and the protective layer. In this manner, the fluorine-containing ether compound represented by Formula (1) can form a lubricating layer having excellent adhesion to the protective layer.

The end group represented by Formula (2) has a 1,2-diol structure (—CH(OH)—CH₂OH) at the end. Therefore, the lubricating layer containing the fluorine-containing ether compound represented by Formula (I) has satisfactory corrosion resistance and can suppress spin-off.

The end group represented by Formula (2) has a divalent organic group having 2 to 30 carbon atoms, which is represented by X. Since the number of carbon atoms in the organic group represented by X is 2 or more, the organic group is moderately rigid, and the intramolecular interaction can be suppressed, and hydrophobicity can be sufficiently obtained, in addition, since the number of carbon atoms is 30 or less, the end group represented by Formula (2) is not extremely bulky, and the movement of the polar group is not hindered so that inhibition of the interaction between the polar group and the protective layer can be suppressed. The number of carbon atoms in the organic group represented by X is preferably in a range of 2 to 15 and more preferably in a range of 3 to 10.

The organic group represented by X has at least one carbon atom that is not bonded to any of the polar group and the ether oxygen atom. Examples of the carbon atom that is not bonded to any of the polar group and the ether oxygen atom include a carbon atom of a methylene group, a methine group, and groups in which these groups are fluorinated, which are not bonded to any of the polar group and the ether oxygen atom. Further, the polar group does not include a fluoro group as described above.

In a case where the organic group represented by X has a polar group having a carbon atom (for example, a carboxy group, a formyl group, a carbonyl group, a cyano group, or a group having an amide bond), the carbon atom of the polar group is not included in “carbon atom that is not bonded to any of the polar group and the ether oxygen atom”.

The number of carbon atoms that are not bonded to any of the polar group and the ether oxygen atom may be 1 or more, and may be, for example, 1 to 6 or 1 to 4. In a case where the number of cardon atoms that are not bonded to any of the polar group and the ether oxygen atom is 1 or more, hydrophobicity of the lubricating layer containing the fluorine-containing ether compound is enhanced. In addition, in a case where the number of carbon atoms that are not bonded to any of the polar group and the ether oxygen atom is 6 or less, the ratio of the number of carbon atoms to the number of polar groups is appropriate, and the polarity of the molecules is appropriate, the ratio of the number of carbon atoms to the number of ether oxygen atoms is appropriate, and thus a fluorine-containing ether compound in which the flexibility of the molecules is appropriate is obtained. In addition, in a case where the compound has a plurality of carbon atoms that are not bonded to any of the polar groups and the ether oxygen atoms, the carbon atoms that are not bonded to any of the polar groups and the ether oxygen atoms may be bonded to each other. In this case, the fluorine-containing ether compound molecules are more rigid, and the intramolecular interaction between the polar groups is suppressed so that the polar groups are more likely to be further involved in the intermolecular interaction between the fluorine-containing ether compounds.

The organic group represented by X may include one or two polar groups. In this case, the adhesion of the fluorine-containing ether compound represented by Formula (1) to the protective layer is improved, and a lubricating layer in which the coating state can be sufficiently obtained even in a case where the thickness thereof is reduced is likely to be formed. In a case where the organic group represented by X contains a polar group, the number of polar groups is preferably 1. In this case, the hydrophilicity of the fluorine-containing ether compound represented by Formula (1) is not extremely high so that water, which causes corrosion, can be further suppressed from being attracted to the layer.

In a case where the organic group represented by X contains a polar group, examples of the polar group include the polar groups included in R¹ and R³ as described above. Among these, a polar group selected from the group consisting of a hydroxy group, a cyano group, and a group having an amide bond is more preferable.

The organic group represented by X in Formula (2) may have 1 to 3 ether oxygen atoms. In this case, the ether oxygen atom imparts moderate flexibility to the fluorine-containing ether compound represented by Formula (1), and increases the affinity between the polar group and the protective layer. In a case where the organic group represented by X has an ether oxygen atom, the number of ether oxygen atoms is preferably 1 or 2. In this case, the fluorine-containing ether compound represented by Formula (1) is not extremely flexible so that the occurrence of the intramolecular interaction can be further suppressed.

The organic group represented by X in Formula (2) may be partially fluorinated. In this case, the hydrophilicity of the organic group represented by X in Formula (2) is decreased as compared with a case where the organic group is not fluorinated. As a result, attraction of water, which causes corrosion, can be suppressed, and thus a fluorine-containing ether compound capable of forming a lubricating layer having more satisfactory corrosion resistance is obtained.

It is preferable that the organic group represented by X in Formula (2) is an acyclic organic group. In a case where the organic group represented by X is acyclic, the organic group is not extremely bulky, the movement of the polar group is not hindered so that the inhibition of the interaction between the polar group and the protective layer can be suppressed as compared with a case where X has a cyclic structure. The acyclic organic group may be linear or branched.

It is preferable that the organic group represented by X in Formula (2) does not have an unsaturated carbon-carbon bond. In a case where the organic group represented by X does not have an unsaturated carbon-carbon bond, the alignment of the molecules is not limited, and the movement of the polar group is not hindered so that the inhibition of the interaction between the polar group and the protective layer can be suppressed as compared with a case where X has an unsaturated carbon-carbon bond.

The end group represented by Formula (2) is preferably a polar group represented by any of Formulae (2-1) to (2-7).

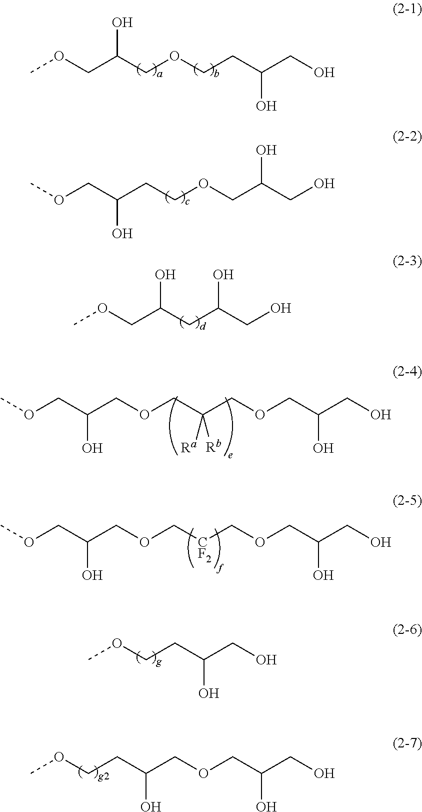

(In Formula (2-1), a represents an integer of 1 to 8, b represents an integer of 1 to 7.)

(In Formula (2-2), c represents an integer of 1 to 7.)

(In Formula (2-3), d represents an integer of 1 to 6.)

(In Formula (2-4), e represents an integer of 1 to 6, e pieces of R^{a}'s and R^{b}'s each independently represent a hydrogen atom or a methyl group.)

(In Formula (2-5), f represents an integer of 1 to 6.)

(In Formula (2-6), g represents an integer of 1 to 6.)

(In Formula (2-7) g2 represents an integer of 1 to 6.)

The end group represented by Formula (2-1) is an end group in which the organic group represented by X in Formula (2) is —CH₂—CH(OH)—(CH₂)ₙ—O—(CH₂)ₙ—CH₂—, a represents an integer of 1 to 8, b represents an integer of 1 to 7.

The end group represented by Formula (2-1) has a structure in which a glycerin structure (—O—CH₂—CH(OH)—CH₂OH) is carbon-increased by b pieces of methylene groups at an end. Since the structure in which the glycerin structure is carbon-increased has moderate rigidity, the formation of the intramolecular interaction by the end group represented by Formula (2-1) can be suppressed. In addition, since the ether bond having a structure in which the glycerin structure is carbon-increased imparts moderate mobility to the end group represented by Formula (2-1), in a case where one hydroxy group of two hydroxy groups in the end portion interacts with the protective layer, the other hydroxy group is more likely to be involved in the intermolecular interaction between the fluorine-containing ether compounds. Therefore, the polar group in the fluorine-containing ether compound represented by Formula (1) can easily form the intermolecular interaction. In this manner, the lubricating layer containing the fluorine-containing ether compound represented by Formula (1) is unlikely to scatter the fluorine-containing ether compound with the rotation of the magnetic recording medium, and has more excellent spin-off resistance.

Since a in Formula (2-1) represents 1 or greater, the end group represented by Formula (2-1) has sufficient hydrophobicity. In addition, since a represents 8 or less, the number of a is not extremely large, the end group represented by Formula (2-1) is not extremely bulky, and the movement of the hydroxy group in the end group represented by Formula (2-1) is not hindered so that inhibition of the interaction with the protective layer can be suppressed, a represents preferably 1 to 6 and more preferably 1 to 4.

Since b in Formula (2-1) represents 1 or greater, the end group represented by Formula (2-1) has sufficient hydrophobicity. In addition, since b represents 7 or less, the number of b is not extremely large, the end group represented by Formula (2-1) is not extremely bulky, and the movement of the hydroxy group in the end group represented by Formula (2-1) is not hindered so that inhibition of the interaction with the protective layer can be suppressed, b represents preferably 1 to 5 and more preferably 1 to 3.

The sum of a and bin Formula (2-1) is preferably 9 or less. In this case, the end group represented by Formula (2-1) is not extremely bulky, and the movement of the hydroxy group in the end group represented by Formula (2-1) is not hindered so that inhibition of the interaction with the protective layer can be further suppressed. The sum of a and b is more preferably 6 or less and still more preferably 4 or less.

The end group represented by Formula (2-2) is an end group in which the organic group represented by X in Formula (2) is —CH₂CH(OH)—CH₂—(CH₂—O—CH₂—, c represents an integer of 1 to 7.

The end group represented by Formula (2-2) has a glycerin structure (—O—CH₂—CH(OH)—CH₂OH) at an end portion. Since the glycerin structure has high mobility, in a case where one hydroxy group of the two hydroxy groups at the end portion interacts with the protective layer, the other hydroxy group is likely to interact with the polar group in the other fluorine-containing ether compound between molecules. Therefore, the polar group in the fluorine-containing ether compound represented by Formula (1) can more easily form an intermolecular interaction. In this manner, the lubricant containing the fluorine-containing ether compound represented by Formula (1) is unlikely to scatter the fluorine-containing ether compound with the rotation of the magnetic recording medium, and has more excellent spin-off resistance.

Since c in Formula (2-2) represents 1 or greater, the end group represented by Formula (2-2) has sufficient hydrophobicity. In addition, since c represents 7 or less, the number of c is not extremely large, the end group represented by Formula (2-2) is not extremely bulky, and the movement of the hydroxy group in the end group represented by Formula (2-2) is not hindered so that inhibition of the interaction with the protective layer can be suppressed, c represents preferably 1 to 5 and more preferably 1 to 3.

The end group represented by Formula (2-3) is an end group in which the organic group represented by X in Formula (2) is —CH₂—CH(OH)—(CH₂)_{d}—, d represents an integer of 1 to 6.

The end group represented by Formula (2-3) does not have an ether oxygen atom, which is a hydrophilic moiety, in X. Therefore, the end group represented by Formula (2-3) has a low affinity for water. Therefore, the lubricating layer containing the fluorine-containing ether compound represented by Formula (1) is unlikely to take in water which causes corrosion, and thus has more satisfactory corrosion resistance.

Since the end group represented by Formula (2-3) does not have an ether oxygen atom in X of Formula (2), and d represents 1 or greater, the end group is moderately rigid. Therefore, the intramolecular interaction between the polar groups is more effectively suppressed, the intermolecular interaction is more easily formed, and a lubricating layer having more excellent spin-off resistance can be formed. In addition, since d represents 6 or less, the number of d is not extremely large, the end group represented by Formula (2.3) is not extremely bulky, and the movement of the hydroxy group in the end group represented by Formula (2-3) is not hindered so that inhibition of the interaction with the protective layer can be suppressed, d represents preferably 1 to 4 and more preferably 2 to 4.

The end group represented by Formula (2-4) is an end group in which the organic group represented by X in Formula (2) is —CH₂—CH(OH)—CH₂—O—CH₂—(CR^{a}R^{b})ₑ—CH₂O—CH₂—, e represents an integer of 1 to 6, e pieces of R^{a}'s and R^{b}'s each independently represent a hydrogen atom or a methyl group.

e pieces of —CR^{a}R^{b}-'s in the end group represented by Formula (2-4) may be any of —CH₂—, −CH(CH₃)—, or —C(CH₃)₂—.

In a case where the organic group represented by X in Formula (2) has a linear structure, that is, in a case where R^{a} and R^{b} represent a hydrogen atom, the end group represented by Formula (2-4) is not extremely bulky as compared with a case where X has a branched structure. Therefore, the movement of the hydroxy group is not hindered so that inhibition of the interaction with the protective layer can be suppressed. As a result, in a case where one hydroxy group of the two hydroxy groups forming the end 1,2-diol structure interacts with the protective layer, the other hydroxy group can more easily form an intermolecular interaction with the polar group in the other fluorine-containing ether compound. In this manner, the lubricant containing the fluorine-containing ether compound represented by Formula (1) is unlikely to scatter the fluorine-containing ether compound with the rotation of the magnetic recording medium, and has more excellent spin-off resistance.

In a case where the organic group represented by X in Formula (2) has a branched structure, that is, −CH(CH₃)— and/or —C(CH₃)₂—, in the end group represented by Formula (2-4), X is moderately rigid, and the intramolecular interaction between the hydroxy groups contained in the end group can be effectively suppressed. Therefore, the polar group in the fluorine-containing ether compound represented by Formula (1) can more easily form an intermolecular interaction. In this manner, the lubricant containing the fluorine-containing ether compound represented by Formula (1) is unlikely to scatter the fluorine-containing ether compound with the rotation of the magnetic recording medium, and has more excellent spin-off resistance.

Since e in Formula (2-4) represents 1 or greater, the end group represented by Formula (2-4) has sufficient hydrophobicity. In addition, since e represents 6 or less, the end group represented by Formula (2-4) is not extremely bulky, and the movement of the hydroxy group is not hindered so that inhibition of the interaction between the hydroxy group and the protective layer can be suppressed, e represents preferably 1 to 4 and more preferably 1 or 2.

The end group represented by Formula (2-5) is an organic group represented by X in formula (2) that is —CH—CH(OH)—CH₂—O—CH—(CF₃)_{f}—CH₂—O—CH₂—, f represents an integer of 1 to 6.

The end group represented by Formula (2-5) has a linear perfluoroalkylene chain having 1 to 6 carbon atoms, which reduces an affinity for water. In a case of having the end group represented by Formula (2-5), the saturated hydrocarbon group having a perfluoroalkylene chain (—CH₂—(CF₂)_{f}—CH₂— in Formula (2-5)) reduces the polarity of the entire molecule and improves the hydrophobicity. In this manner, the affinity between the fluorine-containing ether compound and water, which causes corrosion, is reduced. As a result, the lubricating layer containing the fluorine-containing ether compound represented by Formula (1) suppresses water, which causes corrosion, from being taken into the layer and has further satisfactory corrosion resistance.

Since f in Formula (2-5) represents 1 or greater, the end group represented by Formula (2-5) is moderately rigid, and the intramolecular interaction can be suppressed. In addition, since f represents 6 or less, the end group represented by Formula (2-5) is not extremely bulky, and the movement of the hydroxy group is not hindered so that inhibition of the interaction between the hydroxy group and the protective layer can be suppressed, f in Formula (2-5) represents preferably 1 to 4 and more preferably 2 to 4.

The end group represented by Formula (2-6) is an end group in which the organic group represented by X in Formula (2) is —(CH₂)_{g}CH₂—, g represents an integer of 1 to 6.

The end group represented by Formula (2-6) does not contain a polar group which is a hydrophilic moiety and an ether oxygen atom in X. Therefore, the end group represented by Formula (2-6) has a low affinity for water. Therefore, the lubricating layer containing the fluorine-containing ether compound represented by Formula (1) is unlikely to take in water which causes corrosion, and thus has more satisfactory corrosion resistance.

Since g in Formula (2-6) represents 1 or greater, the end group represented by Formula (2-6) is moderately rigid, and the hydroxy group having a 1,2-diol structure is unlikely to form an intramolecular interaction. In addition, since g in Formula (2-6) represents 6 or less, the end group represented by Formula (2-6) is not extremely bulky, and the movement of the hydroxy group is not hindered so that inhibition of the interaction between the hydroxy group and the protective layer can be suppressed, g represents preferably 1 to 5 and more preferably 1 to 3.

The end group represented by Formula (2-7) is an end group in which the organic group represented by X in Formula (2) is —(CH₂CH—CH(O)—CH₂O—CH₃—, g2 represents an integer of 1 to 6.

The end group represented by Formula (2-7) has a glycerin structure (—O—CH₂—CH(OH)—CH₂OH) at an end portion. Since the glycerin structure has high mobility, in a case where one hydroxy group of the two hydroxy groups at the end portion interacts with the protective layer, the other hydroxy group is likely to interact with the polar group in the other fluorine-containing ether compound between molecules. Therefore, the polar group in the fluorine-containing ether compound represented by Formula (1) can more easily form an intermolecular interaction. In this manner, the lubricant containing the fluorine-containing ether compound represented by Formula (1) is unlikely to scatter the fluorine-containing ether compound with the rotation of the magnetic recording medium, and has more excellent spin-off resistance.

Since g2 in Formula (2-7) represents 1 or greater, the end group represented by Formula (2-7) is moderately rigid, and the hydroxy group having a 1,2-diol structure is unlikely to form an intramolecular interaction. In addition, since g2 in Formula (2-7) represents 6 or less, the end group represented by Formula (2-7) is not extremely bulky, and the movement of the hydroxy group is not hindered so that inhibition of the interaction between the hydroxy group and the protective layer can be suppressed, g2 represents preferably 1 to 5 and more preferably 1 to 3.

Among the end groups represented by Formulae (2-1) to (2-7), the end groups represented by Formulae (2-3), (2-5), and (2-6) have more satisfactory corrosion resistance.

Among the end groups represented by Formulae (2-1) to (2-7), the end groups represented by Formulae (2-1), (2-2), (2-4), and (2-7) each have more satisfactory spin-off resistance in a case where the sum of a and b in Formula (2-1) represent 4 or less, c in Formula (2-2) represents 3 or less, e in Formula (2-4) represents 2 or less, and g2 in Formula (2-7) represents 3 or less.

In the fluorine-containing ether compound represented by Formula (1), R¹ and R³ may be the same as or different from each other. It is preferable that R¹ and R³ are the same as each other. In this case, the fluorine-containing ether compound can be easily and efficiently produced. Further, the expression “R¹ and R³ are the same as each other” denotes that the atom in R¹ and the atom in R³ are symmetrically disposed with respect to —CH₂—R²—CH₂—.

In the fluorine-containing ether compound represented by Formula (1), in a case where R¹ and R³ are different from each other, both R¹ and R³ each independently represent an end group represented by Formula (2), or one of R¹ and R³ may represent an end group represented by Formula (2) and the other may represent an end group that does not correspond to Formula (2). The end group that does not correspond to Formula (2) may be an end group having one to four polar groups and 1 to 50 carbon atoms as described above.

In a case where one of R¹ or R³ represents an end group represented by Formula (2) and the other represents an end group that does not correspond to Formula (2), it is preferable that the end group that does not correspond to Formula 12) is represented by Formula (3),

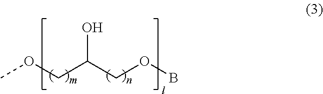

(In Formula (3), 1 represents an integer of 1 to 3. l pieces of m's each independently represent an integer of 1 to 6. l pieces of n's each independently represent an integer of 1 to 6. In one repeating unit, at least one of m or n represents 1. B represents an alkyl group which may have only one polar group, an organic group having one or more carbon-carbon unsaturated bonds, or a hydrogen atom.)

The end group represented by Formula (3) has an oxygen atom (ether oxygen atom) bonded to a methylene group (—CH₂—) bonded to R². Oxygen atoms disposed at the ends of the end group represented by Formula (3) form an ether bond (—O—) with atoms bonded to both sides of the oxygen atoms. This ether bond imparts moderate flexibility to the fluorine-containing ether compound represented by Formula (1), and increases the affinity between the polar group of the end group represented by Formula (3) and the protective layer. In this manner, the fluorine-containing ether compound represented by Formula (1) can form a lubricating layer having excellent adhesion to the protective layer.

1 in Formula (3) represents an integer of 1 to 3, preferably an integer of 1 or 2, and more preferably 1 In a case where 1 in Formula (3) represents 3 or less, the amount of the hydroxy group of the end group represented by Formula (3) is extremely large, and thus water, which causes corrosion, can be prevented from being attracted to the lubricating layer. Therefore, a lubricating layer with satisfactory corrosion resistance can be obtained.

In a case where 1 in Formula (3) represents 2 or 3, combinations of m's and n's in two or three repeating units (—((CH₂)ₙ—CH(OH)—(CH₂)ₙ—O—) may be partially or entirely the same as or different from each other.

l pieces of m's in Formula (3) each independently represent an integer of 1 to 6. l pieces of n's each independently represent an integer of 1 to 6. In one repeating unit (—(CH₂)ₘ—CH(OH)—(CH₂)ₙ—O—) of Formula (3), at least one of m or n represents 1, in this case, the number of carbon atoms in the alkylene group between the carbon atom to which the hydroxy group is bonded and the ether oxygen atom is not extremely increased so that reduction of the mobility of the hydroxy group in the repeating unit can be suppressed.

B in Formula (3) represents an alkyl group which may have only one polar group, an organic group having one or more carbon-carbon unsaturated bonds, or a hydrogen atom.

In a case where B in Formula (3) represents an alkyl group having no polar group, examples of B include a methyl group, an ethyl group, a propyl group, a butyl group, a pentyl group, and a hexyl group.

In a case where B in Formula (3) represents an alkyl group having a polar group, the polar group is preferably any of the preferred examples of the polar group included in R¹ and R³. Among the above-described polar groups, a polar group selected from the group consisting of a hydroxy group, a cyano group, or a group having an amide bond is more preferable.

In a case where B in Formula (3) represents an alkyl group having a polar group, examples of B include a 2-hydroxyethyl group, a 3-hydroxypropyl group, a 4-hydroxybutyl group, a 5-hydroxypentyl group, a 6-hydroxyhexyl group, a 2-aminoethyl group, a 3-aminopropyl group, a 1-carboxyethyl group, a 2-carboxyethyl group, a 3-carboxypropyl group, a 1-carboxyphenethyl group, a 2-carbonylethyl group, a 3-carbonylpropyl group, a 1-acetylmethyl group, a 2-acetylethyl group, a 3-acetylpropyl group, a 2-sulfoethyl group, a 3-sulfopropyl group, a 1-cyanomethyl group, a 2-cyanoethyl group, a 3-cyanopropyl group, a 4-cyanobutyl group, a 2-acetamidoethyl group, a 3-acetamidopropyl group, a 4-acetoamidobutyl group, a 1-carboxamidomethyl group, a 2-carboxyamidoethyl group, a 3-carboxamidopropyl group, and a 4-carboxamidobutyl group.

Among the alkyl groups having the above-described polar group. B represents preferably any of a 2-hydroxyethyl group, a 3-hydroxypropyl group, a 4-hydroxybutyl group, a 5-hydroxypentyl group, a 6-hydroxyhexyl group, a 1-cyanomethyl group, a 2-cyanoethyl group, a 3-cyanopropyl group, a 2-acetamidoethyl group, a 1-carboxamidomethyl group, a 2-carboxyamidoethyl group, or a 3-carboxamidopropyl group and more preferably any of a 2-hydroxyethyl group, a 3-hydroxypropyl group, a 2-cyanoethyl group, a 3-cyanopropyl group, a 2-acetamidoethyl group, or a 1-carboxyamidomethyl group.

In a case where B in Formula (3) represents an organic group having one or more carbon-carbon unsaturated bonds, examples of B include an organic group having at least one selected from an aromatic hydrocarbon, an unsaturated heterocycle, an alkenyl group, and an alkynyl group.

In a case where B in Formula (3) represents an organic group having one or more carbon-carbon unsaturated bonds, examples of B include a phenyl group, a methoxyphenyl group, a fluorinated phenyl group, an acetamidophenyl group, a carboxamidophenyl group, a cyanophenyl group, a naphthyl group, a phenethyl group, a methoxyphenethyl group, a fluorinated phenethyl group, a benzyl group, a methoxybenzyl group, a naphthylmethyl group, a methoxynaphthyl group, a pyrrolyl group, a pyrazolyl group, a methylpyrazolylmethyl group, an imidazolyl group, a furyl group, a furfuryl group, an oxazolyl group, an isoxazolyl group, a thienyl group, a thienylethyl group, a thiazolyl group, a methylthiazolylethyl group, an isothiazolyl group, a pyridyl group, a pyrimidinyl group, a pyridazinyl group, a pyrazinyl group, an indolinyl group, a benzofuranyl group, a benzothienyl group, a benzimidazolyl group, a benzoxazolyl group, a benzothiazolyl group, a benzopyrazolyl group, a benzoisoxazolyl group, a benzoisothiazolyl group, a quinolyl group, an isoquinolyl group, a quinazolinyl group, a quinoxalinyl group, a phthalazinyl group, a cinnolinyl group, a vinyl group, an allyl group, a butenyl group, a propynyl group, a propargyl group, a butynyl group, a methylbutynyl group, a pentynyl group, a methylpentynyl group, and a hexynyl group.

Among the above-described organic groups having one or more carbon-carbon unsaturated bonds, B represents preferably any of a phenyl group, a methoxyphenyl group, an acetamidophenyl group, a carboxamidophenyl group, a cyanophenyl group, a naphthyl group, a phenethyl group, a methoxyphenethyl group, a fluorinated phenethyl group, a thienylethyl group, an allyl group, a butenyl group, or a propargyl group and particularly preferably any of a phenyl group, a methoxyphenyl group, a carboxamidophenyl group, an allyl group, or a butenyl group. In this case, the end group represented by Formula (3) has sufficient hydrophobicity and is not extremely bulky, and the movement of the hydroxy group in Formula (3) is not hindered so that inhibition of the interaction between the hydroxy group and the protective layer can be suppressed.

In a case where B in Formula (3) represents a hydrogen atom, B forms a hydroxy group with the oxygen atom in Formula (3).

It is more preferable that the end group represented by Formula (3) is represented by any of Formulae (3-1) to (3-3).

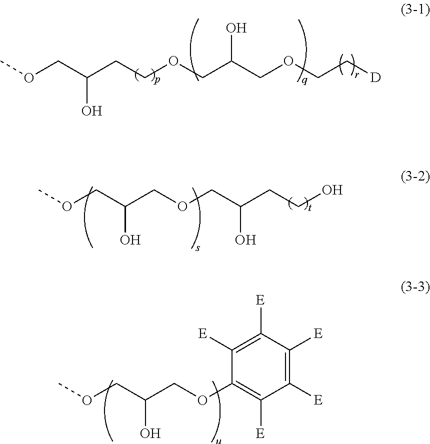

(In Formula (3-1), p represents an integer of 0 to 3, q represents an integer of 0 to 2. r represents an integer of 0 to 5. The total value of p and r is in a range of 1 to 5. D represents a polar group, a vinyl group, an ethynyl group, or an aryl group which may have a substituent.)

(In Formula (3-2), s represents an integer of 0 to 2. t represents an integer of 1 to 5.)

(In Formula (3-3), u represents an integer of 1 to 3. Five H's each independently represent a polar group, an alkoxy group having 1 to 8 carbon atoms, a halogeno group, or a hydrogen atom. Here, in a case where the five E's include a polar group, the number of polar groups in the five E's is 1.)

Each polar group contained in the end group represented by Formulae (3-1) to (3-3) is bonded to different carbon atoms. In addition, carbon atoms to which the polar groups are bonded are bonded to each other through a linking group having a carbon atom to which the polar group is not bonded. Therefore, the end group represented by Formulae (3-1) to (3-3) can be aligned such that both the end polar group and the hydroxy group adjacent to the end polar group can be in close contact with the protective layer by a linking group having a carbon atom to which the polar group is not bonded. Therefore, a lubricating layer in which a strong interaction with the protective layer is obtained can be formed.

In the end group represented by Formulae (3-4) to (3-3), the polar groups in the end group are unlikely to aggregate, and the interaction with the protective layer is likely to occur as compared with the end group in which carbon atoms to which the polar groups are bonded are directly bonded to each other. Therefore, in the end group represented by Formulae (3-1) to (3-3), the end portion in the fluorine-containing ether compound is unlikely to float, and the adhesion to the protective layer is unlikely to be degraded.

In the end group represented by Formula (3-1), a linking group between a carbon atom to which D is bonded and a carbon atom to which a hydroxy group adjacent to D disposed at the end is bonded has an ether oxygen atom. The above-described linking group has a linear structure formed of 2 to 7 atoms including a carbon atom to which a polar group is not bonded.

In the end group represented by Formula (3-1), the linking group has a linear structure formed of two or more atoms including a carbon atom to which a polar group is not bonded. Therefore, the distance between D and the hydroxy group adjacent to D is appropriate. Therefore, since the interaction between D and the hydroxy group adjacent to D in the molecule can be suppressed, the polar group can efficiently adhere to the protective layer. Further, since the end group represented by Formula (3-1) has the above-described structure, even in a case where the end group has an ether oxygen atom, a fluorine-containing ether compound having satisfactory hydrophobicity is obtained. In addition, the molecular mobility is appropriate, the molecular aggregation is unlikely to occur, and thus the adhesion to the protective layer is excellent.

The end group represented by Formula (3-1), has a linear structure formed of 7 or less atoms including a carbon atom to which a polar group is not bonded, in which the above-described linking group has an ether oxygen atom. Therefore, the adhesion between the lubricating layer containing the fluorine-containing ether compound and the protective layer is not impaired due to an extreme increase in hydrophobicity of the linking group.

Therefore, the fluorine-containing ether compound having an end group represented by Formula (3-1) can form a lubricating layer which has excellent adhesion to the protective layer, exhibits high corrosion resistance, and can suppress spin-off.

In the end group represented by Formula (3-1), the total value of p and r is 1 to 5 and preferably 1 to 3. The end group represented by Formula (3-1) has a linking group between a carbon atom to which D is bonded and a carbon atom to which a hydroxy group adjacent to D disposed at the end is bonded. The carbon atom in the above-described linking group prevents the intramolecular interaction between the adjacent polar groups from occurring with a higher priority than the interaction between the polar group and the protective layer. In this manner, the carbon atom in the above-described linking group improves the adhesion between the polar group in Formula (3-1) and the protective layer.

In a case where the number of carbon atoms in the above-described linking group is extremely large, the flexibility of the end group represented by Formula (3-1) is degraded, and thus the entire surface of the protective layer may be difficult to uniformly coat. In the end group represented by Formula (3-1), since the total value of p and r is 5 or ess, the alkylene chain of the main chain portion of Formula (3-1) is not extremely long. Therefore, since the rigid alkylene chain is long, it is possible to prevent degradation of the flexibility of the end portion so that the end portion can be prevented from floating due to a weak interaction with the protective layer. p represents an integer of 0 to 3, preferably 0 or 1, and more preferably 0. r represents an integer of 0 to 5, preferably 1 or 2, and more preferably 1.

D in Formula (3-1) represents a polar group, a vinyl group, an ethynyl group, or an aryl group which may have a substituent. In a case where D represents a polar group, D is preferably any of the preferred examples of the polar group included in R¹ and R³. Among the above-described polar groups, a polar group selected from the group consisting of a hydroxy group, a cyano group, and a group having an amide bond is more preferable. In a case where D represents an aryl group which may have a substituent, an aryl group which may have a substituent contained in the organic group which can be used in a case where B in Formula (3) described above represents an organic group having one or more carbon-carbon unsaturated bonds can be used.

q in Formula (3-1) represents an integer of 0 to 2. The number of polar groups in Formula (3-1) is q+2 in a case where) represents a polar group, and the number thereof is q+1 in a case where 1) represents a vinyl group or an ethynyl group. As described above, the numbers of polar groups included in R¹ and R³ are each preferably 2 or 3. Therefore, q in Formula (3-1) is preferably 0 or 1 in a case where D represents a polar group, and is preferably 1 or 2 in a case where D represents a vinyl group or an ethynyl group. In a case where D represents an aryl group which may have a substituent, it is preferable to select q so that the number of polar groups in Formula (3-1) is 2 or 3.

In the end group represented by Formula (3-2), the linking group between a carbon atom to which the end hydroxy group is bonded and a carbon atom to which a hydroxy group adjacent to the end hydroxy group is bonded does not have an oxygen atom. Therefore, since the intramolecular interaction is small and the intramolecular aggregation is unlikely to occur, the adhesion to the protective layer is excellent.

In the end group represented by Formula (3-2), the linking group has a linear structure formed of 1 to 5 atoms including a carbon atom to which a hydroxy group is not bonded. Since the linking group has a linear structure formed of one or more atoms including a carbon atom to which a hydroxy group is not bonded, a distance between the end hydroxy group and the hydroxy group adjacent to the end hydroxy group is appropriate. Therefore, a fluorine-containing ether compound which is unlikely to cause intramolecular aggregation and has satisfactory hydrophobicity is obtained.

The end group represented by Formula (3-2) has a linear structure formed of 5 or less atoms including a carbon atom to which a hydroxy group is not bonded, in which the above-described linking group does not have an oxygen atom. Therefore, the hydrophobicity of the linking group is not extremely high, the adhesion to the protective layer is not impaired, and the linking group is not extremely bulky so that the influence on the hindrance of movement of the hydroxy group is small.

In the end group represented by Formula (3-2), t represents an integer of 1 to 5. In the end group represented by Formula (3-2), in a case where the number of carbon atoms in the linking group disposed between carbon atoms to which the polar groups are bonded is extremely large as in a case of the end group represented by Formula (3-1), the flexibility of the end group represented by Formula (3-2) is degraded, and thus the entire surface of the protective layer may be difficult to uniformly coat. In the end group represented by Formula (3-2), since t represents 5 or less, the alkylene chain in the main chain portion of Formula (3-2) is not extremely long. Therefore, since the rigid alkylene chain is long, the flexibility of the end portion is not degraded so that it is possible to prevent the interaction between the end hydroxy group and the protective layer from being degraded. t represents preferably 1 or 2 and more preferably 1.

Therefore, since the fluorine-containing ether compound having an end group represented by Formula (3-2) has a linear structure formed of 1 to 5 atoms including a carbon atom to which a hydroxy group is not bonded, in which the above-described linking group in Formula (3-2) does not have an oxygen atom and a hydroxy group is not bonded to a carbon atom, a lubricating layer having excellent adhesion to the protective layer, high corrosion resistance, and suppressed spin-off can be formed.

s in Formula (3-2) represents an integer of 0 to 2. The number of polar groups in Formula (3-2) is s+2, and as described above, the numbers of polar groups included in R¹ and R³ are each preferably 2 or 3. Therefore, it is preferable that s in Formula (3-2) represents 0 or 1.

In the end group represented by Formula (3-3), five E's each independently represent a polar group, an alkoxy group having 1 to 8 carbon atoms, a halogeno group, or a hydrogen atom. In a case where the five E's include a polar group, the number of polar groups in the five E's is 1.

In the end group represented by Formula (3-3), in a case where the five E's include a polar group, the linking group between a carbon atom to which the polar group represented by E is bonded and a carbon atom to which the hydroxy group adjacent to the polar group represented by E is bonded includes an oxygen atom forming an ether bond. The above-described linking group has a structure formed of 3 to 5 atoms including a carbon atom to which the polar group is not bonded. The number of atoms in the above-described linking group denotes the number of atoms in the shortest distance between a carbon atom to which the polar group included in E is bonded and a carbon atom to which a hydroxy group adjacent to the polar group included in E is bonded.

In the end group represented by Formula (3-3), in a case where the five E's include a polar group, the linking group has a structure which has an oxygen atom forming an ether bond and is formed of three or more atoms including a carbon atom to which the polar group is not bonded. Therefore, the distance between the polar group represented by E, and the hydroxy group adjacent to the polar group represented by E is appropriate. In addition, since the benzene ring is rigid, free rotation is difficult to carry out. Therefore, the interaction between the polar group represented by E, which is a substituent bonded to the benzene ring, and the hydroxy group adjacent to the polar group represented by E can be suppressed.

u in Formula (3-3) represents an integer of 1 to 3. In a case where t in Formula (3-3) represents 2 or 3, a linking group between carbon atoms to which a plurality of hydroxy groups adjacent to each other are bonded, included in —(CH₂CH(OH)CH₂O)ₙ—, has a linear structure which has an oxygen atom forming an ether bond and is formed of three atoms having a carbon atom to which a hydroxy group is not bonded. Since the above-described linking group has a linear structure formed of three atoms including a carbon atom to which a hydroxy group is not bonded, the distance between adjacent hydroxy groups is appropriate. Therefore, the interaction between hydroxy groups in the molecule can be suppressed.

Therefore, in the fluorine-containing ether compound having an end group represented by Formula (3-3), the interaction between the polar groups in the molecule can be suppressed. Therefore, the lubricating layer containing the fluorine-containing ether compound having an end group represented by Formula (3-3) has excellent adhesion to the protective layer and has a high spin-off suppression effect.

In the end group represented by Formula (3-3), in a case where the five E's do not include a polar group, the hydrophobicity of the molecule is higher than in a case where one of the five E's is a polar group. Therefore, the lubricating layer containing the fluorine-containing ether compound having an end group represented by Formula (3-3) can further suppress the attraction of water, which causes corrosion, and thus the corrosion resistance is enhanced.

In the end group represented by Formula (3-3), in a case where E represents a polar group, it is preferable that E is any of the preferred examples of the polar group included in R¹ and R³. Among the above-described polar groups. E represents more preferably a polar group selected from the group consisting of a cyano group and a group having an amide bond and still more preferably a cyano group, an acetamido group (—NHC(═O)CH₃), or a carboxamide group (—C(═O)NH₂), in a ease where the polar group is a cyano group, an acetamido group, or a carboxamide group, this is because a fluorine-containing ether compound can form a lubricating layer having stronger interaction with the protective layer. As for another reason, the acidity of the cyano group, the acetamido group, or the carboxamide group is not extremely high, and thus the fluorine-containing ether compound having these groups has almost no influence on the corrosion of the substrate. In a case where E does not represent a polar group, it is preferable that R's each independently represent a methoxy group, a fluoro group, or a hydrogen atom.

The number of polar groups in Formula (3-3) is (u+1) in a case where five E's include a polar group, and the number thereof is u in a case where five E's do not include a polar group. As described above, the numbers of polar groups included in R¹ and R³ are each preferably 2 or 3. Therefore, u in Formula (3-3) represents preferably 1 or 2 in a case where the five E's include a polar group, and is preferably 2 or 3 in a case where the five E's do not include a polar group.

In a case where the five E's in Formula (3-3) include a polar group, four E's other than the polar group may be partially or entirely the same as or different from each other. In a case where the five E's include a polar group, it is preferable that all the four E's other than the polar group are the same as each other. In a case where the five E's do not include a polar group, some or all of the five E's may be the same as or different from each other.

In a case where the five E's in Formula (3-3) include a polar group, the polar group may be at any position of the five E's.

### (PFPE Chain Represented by R²)

In the fluorine-containing ether compound represented by Formula (1). R² represents a perfluoropolyether chain. In a case where the lubricant containing the fluorine-containing ether compound according to the present embodiment is applied onto the protective layer to form a lubricating layer, the surface of the protective layer is coated with the PFPE chain represented by R², lubricity is imparted to the lubricating layer, and a frictional force between the magnetic head and the protective layer is reduced. The PFPE chain represented by R² is appropriately selected according to the performance required for the lubricant containing the fluorine-containing ether compound.

Examples of the PFPE chain represented by R² include a polymer or a copolymer of perfluoroalkylene oxide. Examples of the perfluoroalkylene oxide include perfluoromethylene oxide, perfluoroethylene oxide, perfluoro-n-propylene oxide, perfluoroisopropylene oxide, and perfluorobutylene oxide.

It is preferable that R² in Formula (1) represents a PFPE chain represented by Formula (4) derived from, for example, a polymer or a copolymer of perfluoroalkylene oxide.

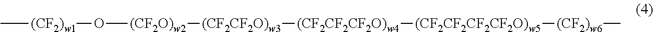

(In Formula (4), w2, w3, w4, and w5 represent an average degree of polymerization, and each independently represent 0 to 20. Here, all of w2, w3, w4, and w5 do not represent 0 at the same time, w1 and w6 represent an average value representing the number of CF₂'s, and each independently represent 1 to 3. An arrangement order of (CF₂O), (F₂CFO), (CF₂CF₂CF₂O), and (CF₂CF₂CF₂CF₂O), which are the repeating units in Formula (4), is not particularly limited.)

In Formula (4), w2, w3, w4, and w5 represent an average degree of polymerization, and each independently represent 0 to 20, preferably 0 to 15, and more preferably 0 to 10.

In Formula (4), w1 and w6 represent an average value indicating the number of CF₂'s, and each independently represent 1 to 3, w1 and w6 are determined depending on the structure of the repeating unit disposed at the end of the chain structure in the PFPE chain represented by Formula (4).

(CF₂O), (CF₂CF₂O), (CF₂CF₂CF₂O), and (CF₂CF₂CF₂CF₂O) in Formula (4) are repeating units. The arrangement order of the repeating units in Formula (4) is not particularly limited. In addition, the number of kinds of repeating units in Formula (4) is not particularly limited.

R² in Formula (1) is preferably any one selected from the PFPE chains represented by Formulae (4-1) to (4-4).

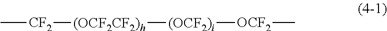

(In Formula (4-1), h and i represent an average degree of polymerization, where h represents 1 to 20 and i represents 0 to 20.)

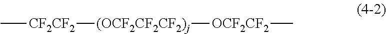

(In Formula (4-2), j represents an average degree of polymerization and represents 1 to 15.)

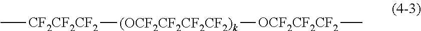

(In Formula (4-3), k represents an average degree of polymerization and represents 1 to 10.)

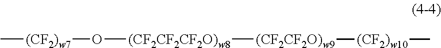

(In Formula (4-4), w8 and w9 represent an average degree of polymerization, and each independently represents 1 to 20, w7 and w10 represent an average value representing the number of CFs, and each independently represent 1 or 2.)

In a case where R² represents any one selected from the PFPE chains represented by Formulae (4-1) to (4-4), the fluorine-containing ether compound is a compound capable of obtaining a lubricating layer having satisfactory lubricity. In addition, in a case where R² represents any one selected from the PFPE chains represented by Formulae (4-1) to (4-4), the ratio of the number of oxygen atoms (number of ether bonds (—O—)) to the number of carbon atoms in the PFPE chain is appropriate. Therefore, the fluorine-containing ether compound has moderate hardness. Therefore, the fluorine-containing ether compound applied onto the protective layer is unlikely to be aggregated on the protective layer, and a lubricating layer having a thinner thickness can be formed with a sufficient coating rate. In addition, the lubricating layer containing the fluorine-containing ether compound in which R² represents any one selected front the PFPE chains represented by Formulae (4-1) to (4-4) is more dense and capable of further suppressing spin-off, which is preferable.

In Formula (4-1), the arrangement order of (OCF₂CF₂) and (OCF₂) which are the repeating units is not particularly limited. In Formula (4-1), the number b of (OCF₂CF₂)'s and the number i of (OCF₂)'s may be the same as or different from each other. The PFPE chain represented by Formula (4-1) may be a polymer of (OCF₂CF₂). In addition, the PFPE chain represented by Formula (4-1) may be any of a random copolymer, a block copolymer, or an alternating copolymer formed of (OCF₂CF₂) and (OCF₂).

In Formulae (4-1) to (4-3), since h representing the average degree of polymerization represents 1 to 20, i represents 0 to 20, j represents 1 to 15, and k represents 1 to 10, a fluorine-containing ether compound capable of obtaining a lubricating layer having satisfactory lubricity is obtained. In addition, in Formulae (4-1) to (4-3), since h and i representing the average degree of polymerization represent 20 or less, j represents 15 or less, and k represents 10 or less, the viscosity of the fluorine-containing ether compound is not extremely high, and a lubricant containing the fluorine-containing ether compound is easy to apply, which is preferable, h, i, j, and k indicating the average degree of polymerization represent preferably 1 to 10, more preferably 1.5 to 8, and still more preferably 2 to 7 from the viewpoint that the fluorine-containing ether compound that is easily wet and spreads on the protective layer and easily obtains a lubricating layer having a uniform film thickness is obtained.

In Formula (4-4), the arrangement order of (CF₂CF₂CF₂O) and (CF₂CF₂O), which are the repeating units, is not particularly limited. In Formula (4-4), the number w8 of (CF₂CF₂CF₂O)'s representing the average degree of polymerization and the number w9 of (CF₂CF₂O)'s may be the same as or different from each other. Formula (4-4) may include any of a random copolymer, a block copolymer, or an alternating copolymer formed of a monomer unit (CF₂CF₂CF₂O) or (CF₂CF₂O).

In Formula (4-4), w8 and w9 representing the average degree of polymerization each independently represent 1 to 20, preferably 1 to 15, and more preferably 1 to 10, w7 and w10 in Formula (4-4) represent an average value indicating the number of CF₂'s, and each independently represent 1 or 2, w7 and w10 are determined according to the structure of the repeating unit or the like disposed at an end of the chain structure in the PFPE chain represented by Formula (4-4).

Specifically, the fluorine-containing ether compound represented by Formula (1) is preferably any of compounds represented by Formulae (AA) to (AS), (BA) to (BH), and (CA) to (CK). In a case where the compound represented by Formula (1) is any of compounds represented by Formulae (AA) to (AS), (BA) to (BH), and (CA) to (CK), a raw material is easily available, and a lubricating layer having more satisfactory corrosion resistance, and is capable of suppressing spin-off even in a case of having a small thickness can be formed.

In the compounds represented by Formulae (AA) to (AS), (BA) to (B), and (CA) to (CK), Rf₁, Rf₂, and Rf₃ representing a PFPE chain each have the following structures. That is, in the compounds represented by Formulae (AA) to (AS). (BA) to (BF), (CA), (CB), and (CE), Rf₁ represents a PFPE chain represented by Formula (4-1). In the compounds represented by Formulae (B). (CC), and (CF) to (CK). Rf₁ represents a PFPE chain represented by Formula (4-2). In the compounds represented by Formulae (BH) and (CD). Rf₁ represents a PFPE chain represented by Formula (4-3). Further, in Formulae (AA) to (AS). (BA) to (BH), and (CA) to (CK), h and i in Rf₁, j in Rf₂, and k in Rf₃ representing a PFPE chain are values indicating an average degree of polymerization, and thus am not necessarily integers.

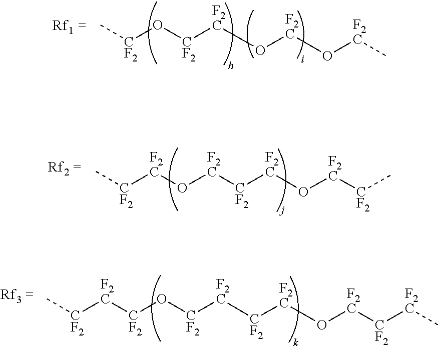

The compound represented by any of Formulae (AA) to (AS) is a compound in which R¹ and R³ in Formula (1) represent an end group represented by any of Formulae (2-1) to (2-6). R² represents a PFPE chain represented by Formula (4-1).

The compound represented by any of Formulae (BA) to (BE) is a compound in which one of R¹ or R³ in Formula (1) represents an end group represented by Formula (2-1) and the other represents an end group represented by any of Formulae (3-1) and (3-2). R² represents a PFPE chain represented by Formula (4-1).

The compound represented by Formula (BF) is a compound in which one of R¹ or R³ in Formula (1) represents an end group represented by Formula (2-1) and the other represents an end group represented by Formula (3). R² represents a PFPE chain represented by Formula (4-1). The end group represented by Formula (3) does not correspond to any of Formulae (3-1) to (3-3).

The compound represented by any of Formulae (BU) and (BH) is a compound in which R¹ and R³ in Formula (1) represents an end group represented by Formula (2-1). R² represents a PFPE chain represented by Formula (4-2) or (4-3).

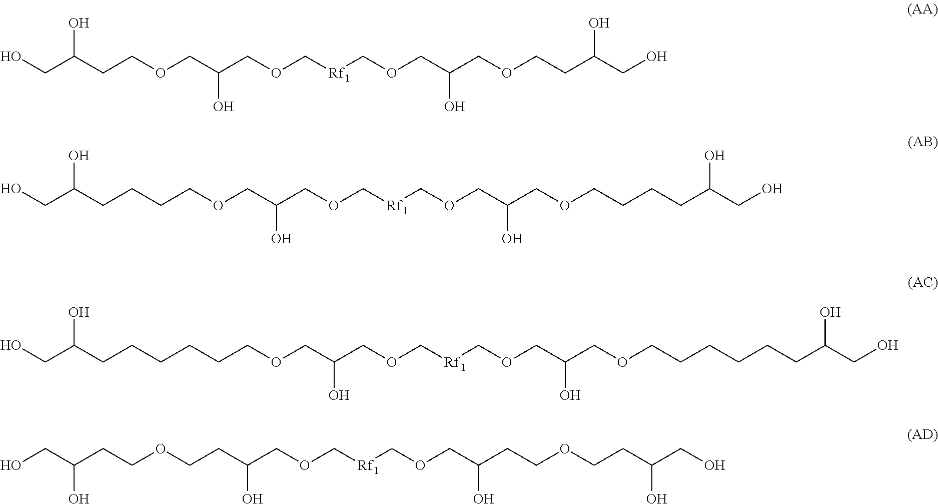

(In Rf₁ in Formula (AA), h and i represent an average degree of polymerization, where h represents 1 to 20 and i represents 0 to 20.)

(In Rf₁ in Formula (AB), h and i represent an average degree of polymerization, where represents 1 to 20 and i represents 0 to 20.)

(In Rf₁ in Formula (AC), h and i represent an average degree of polymerization, where h represents 1 to 20 and i represents 0 to 20.)

(In Rf₁ in Formula (AD), h and i represent an average degree of polymerization, where h represents 1 to 20 and i represents 0 to 20.)

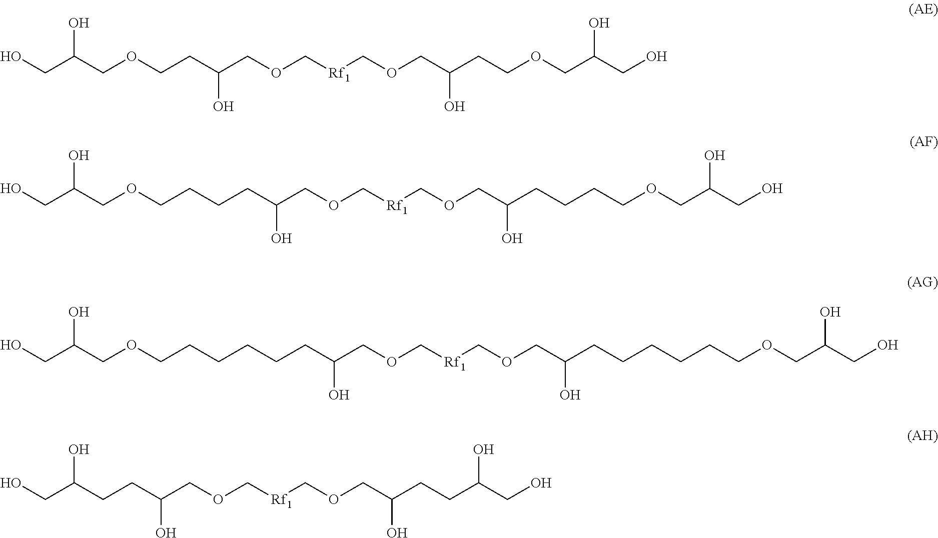

(In Rf₁ in Formula (AE), h and i represent an average degree of polymerization, where h represents 1 to 20 and i represents 0 to 20.)

(In Rf_{f} in Formula (AF), h and i represent an average degree of polymerization, where h represents 1 to 20 and i represents 0 to 20.)

(In Rf₁ in Formula (AG), h and i represent an average degree of polymerization, where h represents 1 to 20 and i represents 0 to 20.)

(In Rf₁ in Formula (AH), h and i represent an average degree of polymerization, h represents 1 to 20, and i represents 0 to 20.)

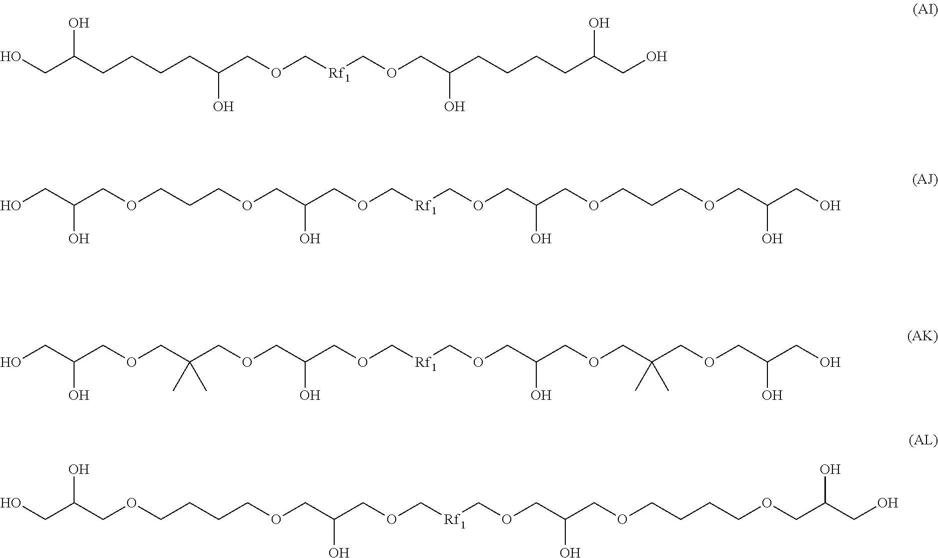

(In Rf₁ in Formula (AI), h and i represent an average degree of polymerization, where h represents 1 to 20 and i represents 0 to 20.)

(In Rf₁ in Formula (AJ), h and i represent an average degree of polymerization, where h represents 1 to 20 and i represents 0 to 20.)

(In Rf₁ in Formula (AK), h and i represent an average degree of polymerization, where h represents 1 to 20 and i represents 0 to 20.)

(In Rf₁ in Formula (AL), h and i represent an average degree of polymerization, where h represents 1 to 20 and i represents 0 to 20.)

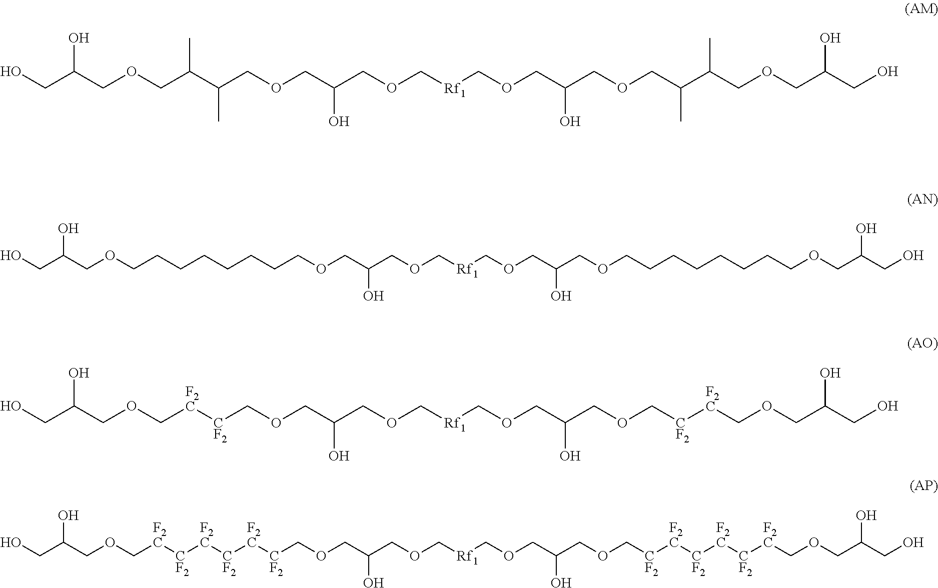

(In Rf₁ in Formula (AM), h and i represent an average degree of polymerization, where h represents 1 to 20 and i represents 0 to 20.)

(In Rf₁ in Formula (AN), h and i represent an average degree if polymerization, where h represents 1 to 20 and i represents 0 to 20.)

(In Rf₁ in Formula (AO), h and i represent an average degree of polymerization, where h represents 1 to 20 and i represents 0 to 20.)

(In Rf₁ in Formula (AP), h and i represent an average degree of polymerization, where h represents 1 to 20 and i represents 0 to 20.)

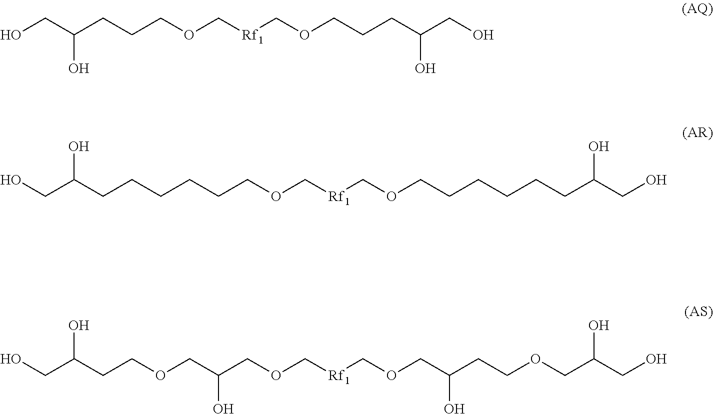

(In Rf₁ in Formula (AQ), h and i represent an average degree of polymerization, where h represents 1 to 20 and i represents 0 to 20.)

(In Rf₁ in Formula (AR), h and i represent an average degree of polymerization, where h represents 1 to 20 and i represents 0 to 20.)

(In Rf₁ in Formula (AS), h and i represent an average degree of polymerization, where h represents 1 to 20 and i represents 0 to 20.)

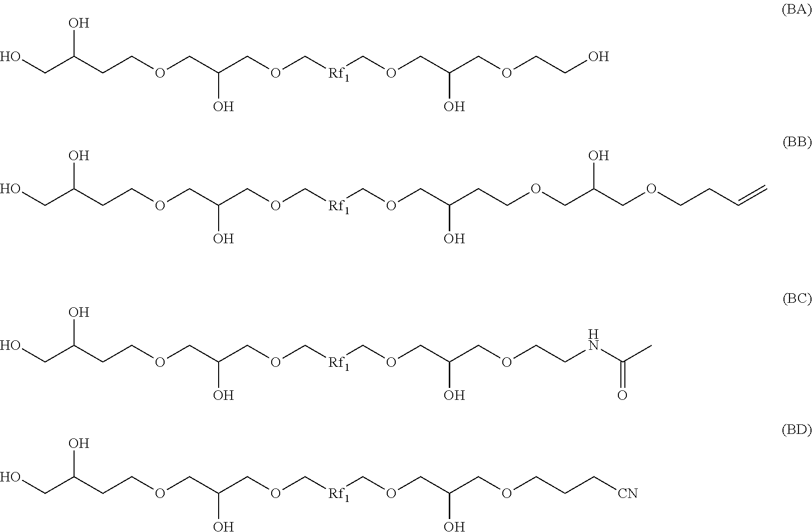

(In Rf₁ of Formula (BA), h and i represent an average degree of polymerization, where h represents 1 to 20 and i represents 0 to 20.)

(In Rf₁ in Formula (BB), h and i represent an average degree of polymerization, where h represents 1 to 20 and i represents 0 to 20.)

(In Rf₁ in Formula (BC), h and i represent an average degree of polymerization, where h represents 1 to 20 and i represents 0 to 20.)

(In Rf₁ in Formula (BD), h and i represent an average degree of polymerization, where h represents 1 to 20 and i represents 0 to 20.)

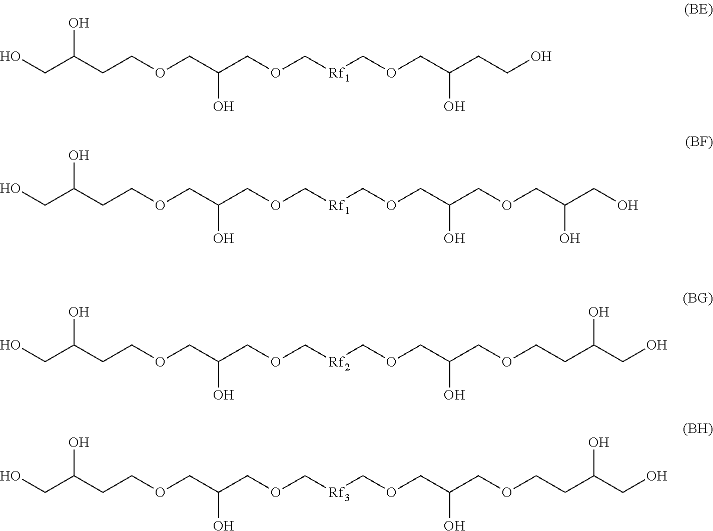

(In Rf₁ in Formula (BE), h and i represent an average degree of polymerization, where h represents 1 to 20 and i represents 0 to 20.)

(In Rf₁ in Formula (BF), h and i represent an average degree of polymerization, where h represents 1 to 20 and i represents 0 to 20.)

(In Rf₂ in Formula (BG), j represents an average degree of polymerization and represents 1 to 15.)

(In Rf₁ in Formula (BH), k represents an average degree of polymerization and represents 1 to 10.)

The compound represented by any of Formulae (CA) to (CK) is a compound in which R¹ and R³ in Formula (1) represent an end group represented by any of Formulae (2-1) to (2-7). R² represents a PFPE chain represented by any of Formulae (4-1) to (4-3).

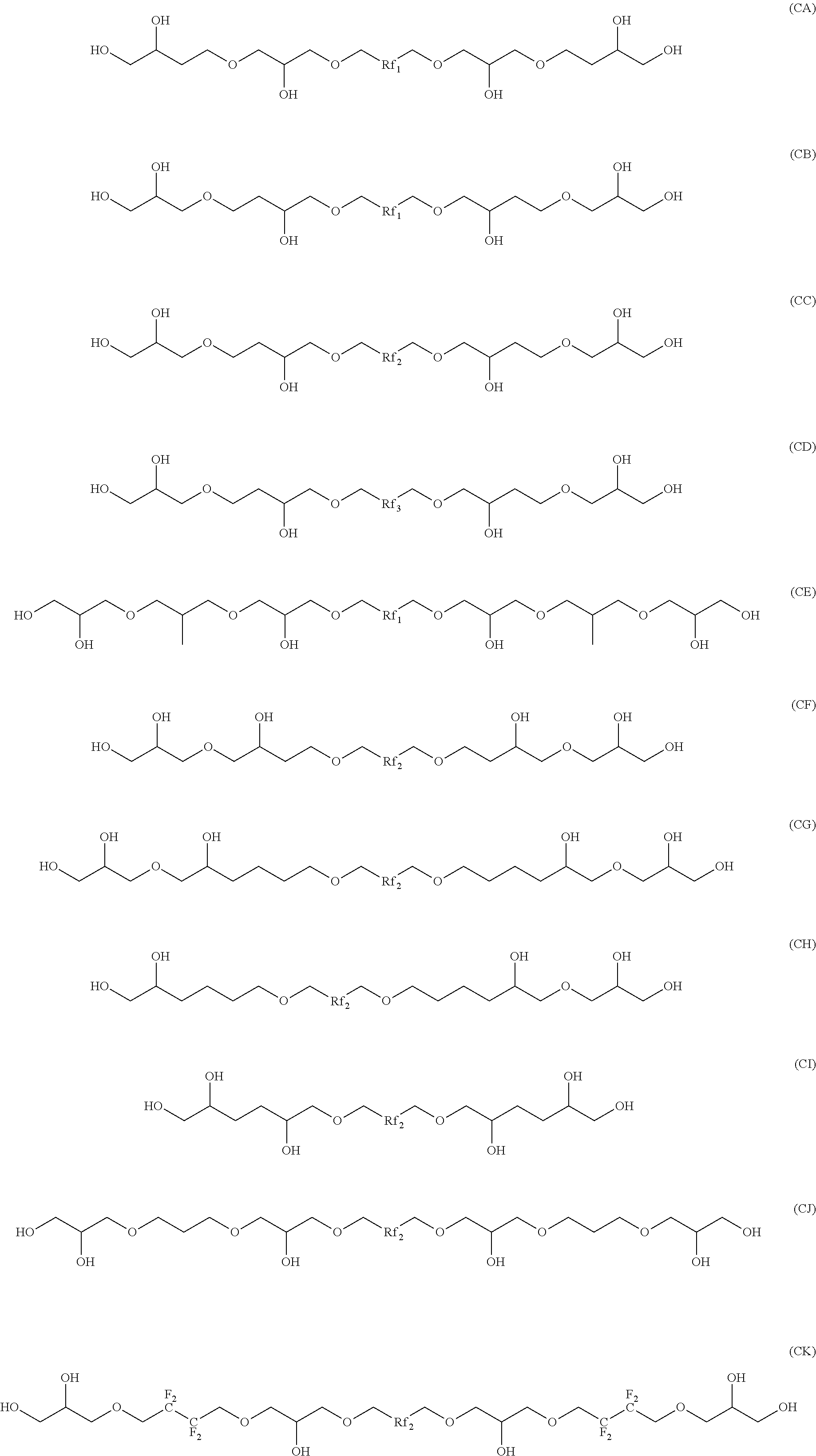

(In Rf₁ in Formula (CA), h and i represent an average degree of polymerization, where h represents 1 to 20 and i represents 0.)

(In Rf₁ of Formula (CB), h and i represent an average degree of polymerization, where h represents 1 to 20 and i represents 0).

(In Rf₂ in Formula (CC), j represents an average degree of polymerization, and represents 1 to 15.)

(In Rf in Formula (CD), k represents an average degree of polymerization and represents 1 to 10.)

(In Rf₁ in Formula (CE), h and i represent an average degree of polymerization, where h represents 1 to 20 and i represents 0 to 20.)

(In Rf₂ in Formula (CF), j represents an average degree of polymerization, and represents 1 to 15.)

(In Rf₂ in Formula (CG), j represents an average degree of polymerization and represents 1 to 15.)

(In Rf₂ in Formula (CH), j represents an average degree of polymerization, and represents 1 to 15.)

(In Rf₂ in Formula (CI), j represents an average degree of polymerization and represents 1 to 15.)

(In Rf₂ in Formula (CJ), j represents an average degree of polymerization and represents 1 to 15.)

(In Rf₂ in Formula (C), j represents an average degree of polymerization, and represents 1 to 15.)

The number-average molecular weight (Mn) of the fluorine-containing ether compound of the present embodiment is preferably in a range of 500 to 10,000) and particularly preferably in a range of 1,000 to 5,000. In a case where the number-average molecular weight thereof is 500 or greater, the lubricating layer formed of the lubricant containing the fluorine-containing ether compound according to the present embodiment has excellent heat resistance. The number-average molecular weight of the fluorine-containing ether compound is more preferably 1000 or greater. In addition, in a case where the number-average molecular weight thereof is 10000 or less, the viscosity of the fluorine-containing ether compound is appropriate, and a lubricating layer with a small film thickness can be easily formed by applying a lubricant containing the fluorine-containing ether compound. The number-average molecular weight of the fluorine-containing ether compound is preferably 5,000 or less from the viewpoint that the viscosity is easy to handle in a case where the fluorine-containing ether compound is applied to a lubricant.

The number-average molecular weight (Mn) of the fluorine-containing ether compound is a value measured by ¹H-NMR and ¹⁹F-NMR using AVANCE III 400 (manufactured by Bruker BioSpin GmbH). Specifically, the number of repeating units of the PFPE chain is calculated from the integrated value measured by ¹⁹F-NMR, and the number-average molecular weight is determined. In the measurement of nuclear magnetic resonance (NMR), the sample is diluted with a solvent of hexafluorobenzene/d-acetone (4/1 v/v) and measured. The peak of hexafluorobenzene is set to −164.7 ppm as the reference of the ¹⁹F-NMR chemical shift, and the peak of acetone is set to 2.2 ppm as the reference of the ¹H-NMR chemical shift.

In the fluorine-containing ether compound of the present embodiment, it is preferable that the polydispersity (ratio of weight-average molecular weight (Mw) to number-average molecular weight (Mn)) is set to 1.3 or less by fractionating the molecular weight by an appropriate method. In the present embodiment, a method for fractionating the molecular weight is not particularly limited, and for example, molecular weight fractionation by a silica gel column chromatography method, a gel permeation chromatography (GPC) method, or the like, molecular weight fractionation by a supercritical extraction method, and the like can be used.

### “Production Method”

A method for producing the fluorine-containing ether compound of the present embodiment is not particularly limited, and the fluorine-containing ether compound can be produced by a known production method of the related art. The fluorine-containing ether compound of the present embodiment can be produced, for example, by the following production method.

(In Case where R¹ and R³ are the Same as Each Other)

In order to produce a compound in which R¹ and R³ in Formula (1) are the same as each other, first, a fluorine-based compound in which hydroxymethyl groups (—CH₂OH) are disposed at both ends of the perfluoropolyether chain corresponding to R² in Formula (1) is prepared.

Next, a hydroxy group of a hydroxymethyl group disposed at both ends of the fluorine-based compound reacts with an epoxy group of an epoxy compound having a group as R¹ in Formula (1) (group as R³). In this manner, a compound having a group corresponding to R¹ (=group corresponding to R³) at both ends of the perfluoropolyether chain corresponding to R² is obtained.

As the epoxy compound having a group as R¹ in Formula (1) (=group as R³), for example, compounds represented by Formulae (5-1) to (5-16) and Formula (5-24) can be used.

In the above-described reaction, a compound having a group as R¹ in Formula (1) (=group as R³) and a leaving group (hereinafter, also simply referred to as “compound having a leaving group”) may be used instead of the epoxy compound. As the compound having a leaving group, compounds represented by Formulae (5-25) to (5-27) can be used. MOM in Formula (5-25) and Formula (5-26) represents a methoxymethyl group. In Formulae (5-25) to (5-27), Ts represents a tosyl group.

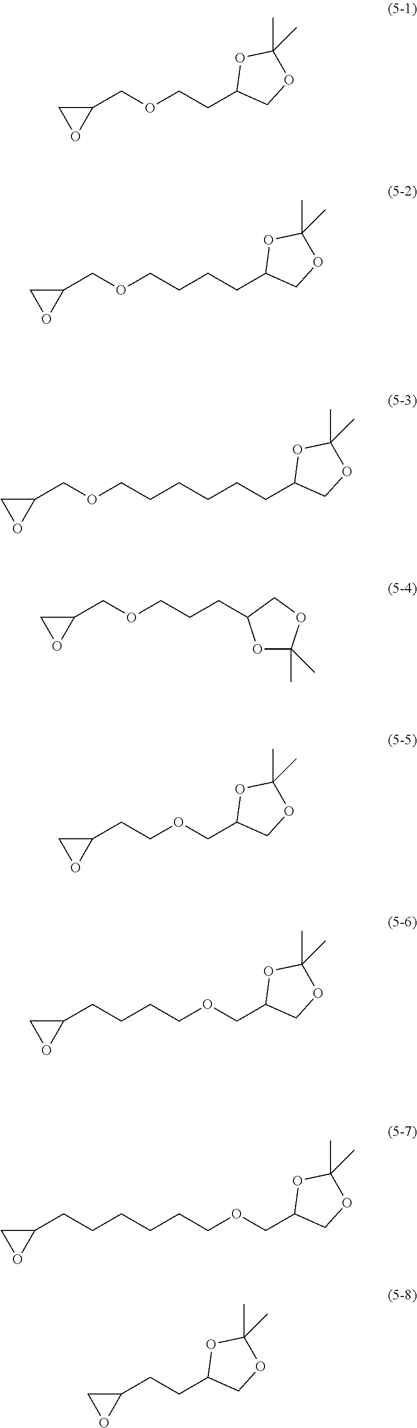

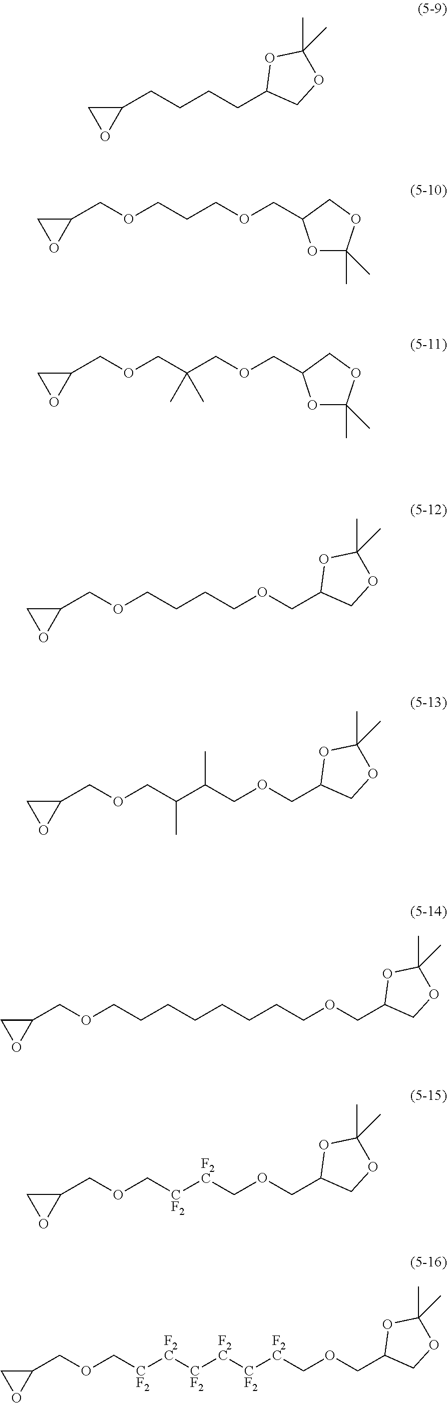

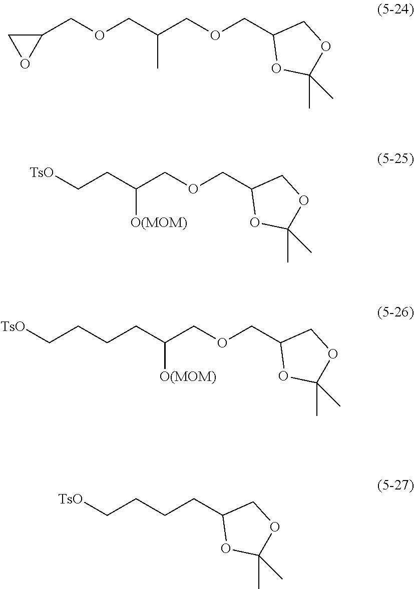

In a case where the fluorine-based compound reacts with the epoxy compound (or the compound having a leaving group), the hydroxy group of the epoxy compound (or the compound having a leaving group) may be protected with an appropriate protective group, and then may react with the fluorine-based compound.

The epoxy compound (for example, an epoxy compound represented by Formula (5-1)) can be produced, for example, by a method of reacting an alcohol compound which may be appropriately protected with a halogen compound having an epoxy group, as shown in Formula (6-1).

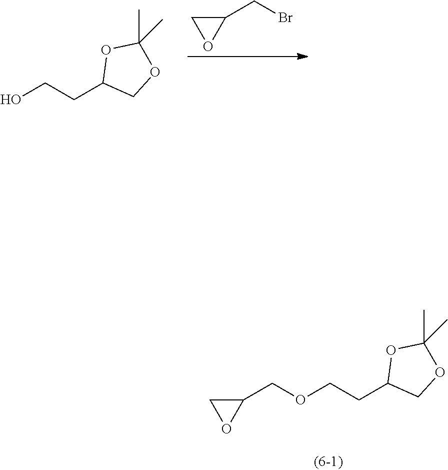

The epoxy compound (for example, an epoxy compound represented by Formula (5-5)) may be produced, for example, by a method of reacting an alcohol compound having a vinyl group with a halogen compound having an alcohol which may be appropriately protected, and then oxidizing the obtained compound by the reaction of m-chloroperbenzoic acid (mCPBA), as shown in Formula (6-2).

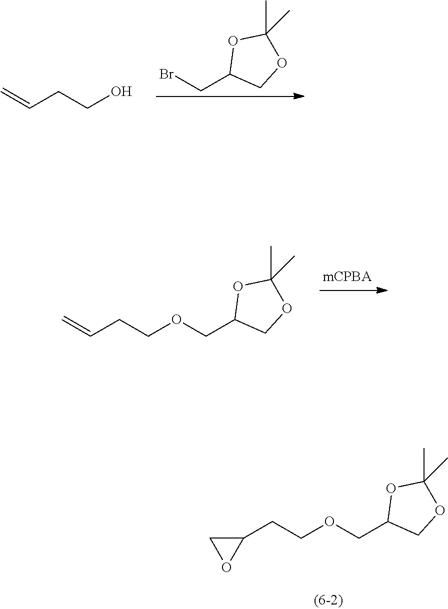

The epoxy compound (for example, an epoxy compound represented by Formula (5-100 may be produced, for example, by a method of sequentially reacting a halogen compound having an alcohol which may be appropriately protected and a halogen compound having an epoxy group with the diol compound, as shown in the following Formula (6-3).

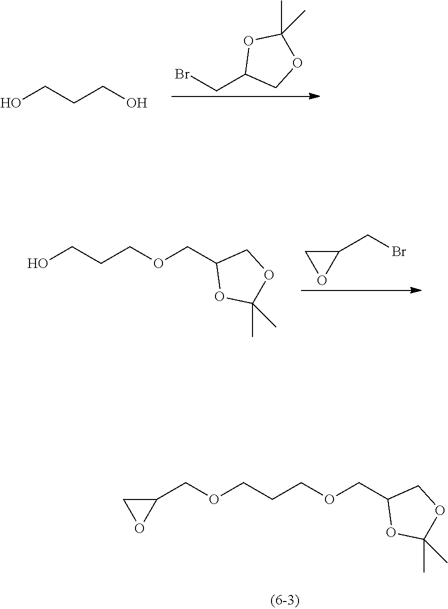

A commercially available product may be purchased and used as the epoxy compound (or a compound having a leaving group).

By performing the above-described step, a compound in which R¹ and R³ in Formula (M) are the same as each other is obtained.

In Formula (1), in order to produce a compound in which R¹ and R³ are the same as each other, the following method may be used.

First, a fluorine-based compound in which a hydroxymethyl group (—CH₂OH) is disposed at both ends of a perfluoropolyether chain corresponding to R² in Formula (1) is prepared. A hydroxy group of a hydroxymethyl group disposed at both ends of the fluorine-based compound reacts with a halogen compound having a vinyl group at an end. In this manner, a compound having a vinyl group is obtained at both ends of the chain structure having a perfluoropolyether chain.

Subsequently, the vinyl groups at both ends of the obtained compound are oxidized to obtain a compound having an epoxy group at both ends of a chain structure having a perfluoropolyether chain. The epoxy groups at both ends are converted into 1,2-diols by being subjected to a ring-opening reaction. By performing the above-described step, a compound in which R¹ and R³ in Formula (1) are the same as each other is obtained.

(In Case where R¹ and R³ are Different from Each Other)

In Formula (1), in order to produce a compound in which R¹ and R³ are different from each other, first, a fluorine-based compound in which a hydroxymethyl group (—C₂H₂OH) is disposed at both ends of a perfluoropolyether chain corresponding to R² in Formula (1) is prepared.

Next, the hydroxy group of the hydroxymethyl group disposed at one end of the fluorine-based compound reacts with the epoxy group of the epoxy compound having the group represented by R¹ in Formula (1). In this manner, an intermediate compound 1 having a group corresponding to R¹ at one end of the perfluoropolyether chain corresponding to R² is obtained (first reaction).

Next, the above-described intermediate compound 1 reacts with the epoxy compound having a group represented by R³ in Formula (I) (second reaction).

In the first reaction and/or the second reaction, a compound having a leaving group may be used instead of the epoxy compound.

In a case where R⁴ and R³ are different from each other, one of R¹ and R³ represents an end group represented by Formula (2), and the other represents an end group that does not correspond to Formula (2), for example, an epoxy compound represented by any of Formulae (5-17) to (5-23) can be used as the epoxy compound corresponding to the end group that does not correspond to Formula (2). In Formulae (5-17), (5-18), and (5-21) to (5-23), THP represents a tetrahydropyranyl group.

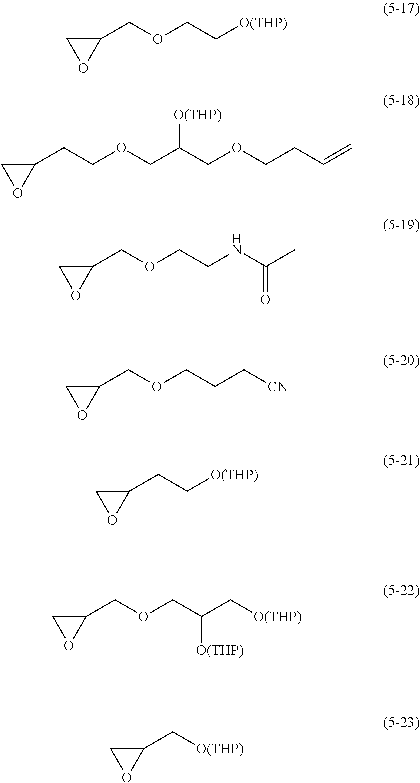

By performing the above-described step, a compound in Formula (I), in which R¹ and R³ are different from each other, is obtained.

### [Lubricant for Magnetic Recording Medium]

A lubricant for a magnetic recording medium according to the present embodiment contains the fluorine-containing ether compound represented by Formula (1).

In the lubricant of the present embodiment, as long as the characteristics are not impaired by containing the fluorine-containing ether compound represented by Formula (1), known materials used as a material for the lubricant can be used in the form of a mixture as necessary.

Specific examples of the known materials include, for example, FOMBLIN (registered trademark) ZDIAC, FOMBLIN ZDEAL, and FOMBLAN AM-2001 (all manufactured by Solvay Solexis), and Moresco A20H (manufactured by Moresco). The known materials used in the form of a mixture with the lubricant according to the present embodiment has a number-average molecular weight of preferably 1000 to 10000.

In a case where the lubricant, of the present embodiment contains other materials of the fluorine-containing ether compound represented by Formula (1), the content of the fluorine-containing ether compound represented by Formula (1) in the lubricant of the present embodiment is preferably 70% by mass or greater, more preferably 90% by mass or greater, and still more preferably 95% by mass or greater.

Since the lubricant of the present embodiment contains the fluorine-containing ether compound represented by Formula (1), a lubricating layer having excellent corrosion resistance and capable of suppressing spin-off can be formed.

### [Magnetic Recording Medium]

The magnetic recording medium according to the present embodiment is formed by sequentially providing at least a magnetic layer, a protective layer, and a lubricating layer on a substrate.

In the magnetic recording medium according to the present embodiment, one or two or more underlayers can be provided between the substrate and the magnetic layer as necessary. In addition, at least one of an adhesive layer or a soft magnetic layer can be provided between the underlayer and the substrate.

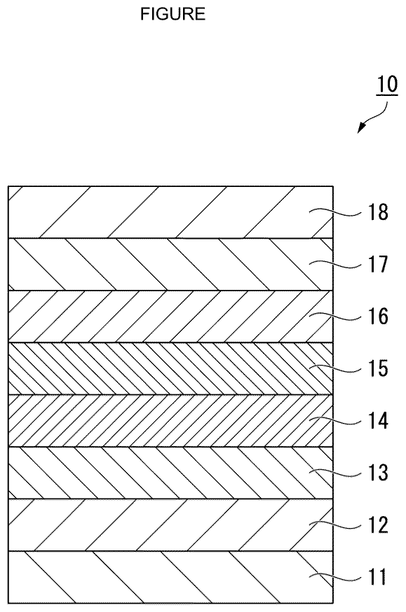

FIG. 1 is a schematic cross-sectional view showing an embodiment of the magnetic recording medium according to the present invention.

The magnetic recording medium 10 according to the present embodiment has a structure in which an adhesive layer 12, a soft magnetic layer 13, a first underlayer 14, a second underlayer 15, a magnetic layer 16, a protective layer 17, and a lubricating layer 18 are sequentially provided on a substrate 11.

### “Substrate”

As the substrate 11, for example, a non-magnetic substrate on which a film made of NiP or a NiP alloy is formed on a base made of a metal or an alloy material such as Al or an Al alloy can be used.

In addition, as the substrate 11, a non-magnetic substrate made of a non-metal material such as glass, ceramics, silicon, silicon carbide, carbon, or a resin may be used, or a non-magnetic substrate in which a film of NiP or a NiP alloy is formed on a base made of these non-metal materials may be used.

### “Adhesive Layer”

The adhesive layer 12 prevents the progression of corrosion of the substrate 11 in a case where the substrate 11 and the soft magnetic layer 13 provided on the adhesive layer 12 are disposed in contact with each other.

The material of the adhesive layer 12 can be appropriately selected from, for example, Cr, a Cr alloy, Ti, a Ti alloy, CrTi, NiAl, and an AlRu alloy. The adhesive layer 12 can be formed by, for example, a sputtering method.

### “Soft Magnetic Layer”

It is preferable that the soft magnetic layer 13 has a structure in which a first soft magnetic film, an intermediate layer formed of a Ru film, and a second soft magnetic film are sequentially laminated. That is, it is preferable that the soft magnetic layer 13 has a structure in which the upper and lower soft magnetic films of the intermediate layer are antiferro-coupled (AFC) by interposing the intermediate layer formed of a Ru film between the two layers of the soft magnetic films.

Examples of the material of the first soft magnetic film and the second soft magnetic film include a CoZrTa alloy and a CoFe alloy.

It is preferable to add any of Zr, Ta, or Nb to the CoFe alloy used in the first soft magnetic film and the second soft magnetic film. In this manner, the amorphization of the first soft magnetic film and the second soft magnetic film is promoted. As a result, it is possible to improve the aligning properties of the first underlayer (seed layer) and to reduce the floating amount of the magnetic head.

The soft magnetic layer 13 can be formed by, for example, a sputtering method.

### “First Underlayer”

The first underlayer 14 is a layer that controls the alignment and the crystal size of the second underlayer 15 and the magnetic layer 16 provided thereon.

Examples of the first underlayer 14 include a Cr layer, a Ta layer, a Ru layer, a CrMo alloy layer, a CoW alloy layer, a CrW alloy layer, a CrV alloy layer, and a CrTi alloy layer.

The first underlayer 14 can be formed by, for example, a sputtering method.

### “Second Underlayer”

The second underlayer 15 is a layer in which the alignment of the magnetic layer 16 is controlled to be satisfactory. The second underlayer 15 is preferably a layer formed of Ru or a Ru alloy.

The second underlayer 15 may consist of a single layer or a plurality of layers. In a case where the second underlayer 15 consists of a plurality of layers, all the layers may be formed of the same material, or at least one layer may be formed of a different material.

The second underlayer 15 can be formed by, for example, a sputtering method.

### “Magnetic Layer”

The magnetic layer 16 is formed of a magnetic film in which a magnetization easy axis is oriented in a direction perpendicular or horizontal to the substrate surface. The magnetic layer 16 is a layer containing Co and Pt. The magnetic layer 16 may be a layer containing an oxide, Cr, B, Cu, Ta, Zr, or the like in order to improve the SNR characteristics.

Examples of the oxide contained in the magnetic layer 16 include SiO₂, SiO, Cr₂O₃, CoO, Ta₂O₃, and TiO₂.

The magnetic layer 16 may consist of one layer or a plurality of magnetic layers formed of materials having different compositions.

For example, in a case where the magnetic layer 16 consists of three layers of a first magnetic layer, a second magnetic layer, and a third magnetic layer which are laminated in this order from the bottom, it is preferable that the first magnetic layer has a granular structure formed of a material containing Co, Cr, and Pt and further containing an oxide. As the oxide contained in the first magnetic layer, for example, an oxide of Cr, Si, Ta, Al, Ti, Mg, or Co is preferably used. Among these, TiO₂, Cr₂O₃, SiO₂, or the like can be particularly suitably used. In addition, it is preferable that the first magnetic layer is formed of a composite oxide to which two or more kinds of oxides are added. Among these, Cr₂O₃—SiO₂, Cr₂O₃—TiO₂, SiO₂—TiO₂, or the like can be particularly suitably used. The first magnetic layer can contain one or more kinds of elements selected from B, Ta, Mo, Cu, Nd, W, Nb, Sm, Tb, Ru, and Re in addition to Co, Cr, Pt, and an oxide.

The same material as that of the first magnetic layer can be used for the second magnetic layer. It is preferable that the second magnetic layer has a granular structure.

It is preferable that the third magnetic layer has a non-granular structure formed of a material containing Co, Cr, and Pt and not containing an oxide. The third magnetic layer can contain one or more elements selected from B, Ta, Mo, Cu, Nd, W, Nb, Sm, Tb, Ru, Re, and Mn in addition to Co, Cr, and Pt.

In a case where the magnetic layer 16 is formed of a plurality of magnetic layers, it is preferable to provide a non-magnetic layer between the adjacent magnetic layers. In a case where the magnetic layer 16 is formed of three layers of a first magnetic layer, a second magnetic layer, and a third magnetic layer, it is preferable to provide a non-magnetic layer between the first magnetic layer and the second magnetic layer and between the second magnetic layer and the third magnetic layer.

As the non-magnetic layer provided between adjacent magnetic layers of the magnetic layer 16, for example, Ru, a Ru alloy, a CoCr alloy, or a CoCrX1 alloy (X1 represents one or two or more kinds of elements selected from Pt, Ta, Zr, Re, Ru, Cu, Nb, Ni, Mn, Ge, Si, O, N, W, Mo, Ti, V, and B) can be suitably used.

It is preferable that, as the non-magnetic layer provided between adjacent magnetic layers of the magnetic layer 16, an alloy material containing an oxide, a metal nitride, or a metal carbide is used. Specifically, for example, SiO₂, Al₂O₃, Ta₂O₅, Cr₂O₃, MgO, Y₂O₃, or TiO₂ can be used as the oxide. As the metal nitride, for example, AlN, Si₃N₄, TaN, or CrN can be used. As the metal carbide, for example, TaC, BC, or SiC can be used.

The non-magnetic layer can be formed by, for example, a sputtering method.

In order to achieve a higher recording density, it is preferable that the magnetic layer 16 is a magnetic layer for perpendicular magnetic recording in which a magnetization easy axis is oriented in a direction perpendicular to the substrate surface. The magnetic layer 16 may be a magnetic layer for in-plane magnetic recording.

The magnetic layer 16 may be formed by any of the known methods in the related art, such as an evaporation method, an ion beam sputtering method, and a magnetron sputtering method. The magnetic layer 16 is usually formed by a sputtering method

### “Protective Layer”

The protective layer 17 protects the magnetic layer 16. The protective layer 17 may be formed of one layer or a plurality of layers. As the protective layer 17, a carbon-based protective layer can be preferably used, and an amorphous carbon protective layer is particularly preferable. In a case where the protective layer 17 is a carbon-based protective layer, the interaction with the polar group (particularly, the hydroxy group) contained in the fluorine-containing ether compound in the lubricating layer 18 is further enhanced, which is preferable.

The adhesion force between the carbon-based protective layer and the lubricating layer 18 can be controlled by forming the carbon-based protective layer with hydrocarbon and/or nitrogenated carbon and adjusting the hydrogen content and/or the nitrogen content in the carbon-based protective layer. The hydrogen content in the carbon-based protective layer is preferably in a range of 3% by atom to 20% by atom in a case of being measured by a hydrogen forward scattering method (HFS). In addition, the nitrogen content in the carbon-based protective layer is preferably in a range of 4% by atom to 15% by atom in a case of being measured by X-ray photoelectron spectroscopy (XPS).

The hydrogen and/or nitrogen contained in the carbon-based protective layer are not necessarily uniformly contained in the entire carbon-based protective layer. It is suitable that the carbon-based protective layer is, for example, a composition gradient layer in which nitrogen is contained on the lubricating layer 18 side of the protective layer 17 and hydrogen is contained on the magnetic layer 16 side of the protective layer 17. In this case, the adhesion force between the magnetic layer 16, the lubricating layer 18, and the carbon-based protective layer is further improved.

The film thickness of the protective layer 17 is preferably in a range of 1 nm to 7 nm. In a case where the film thickness of the protective layer 17 is 1 nm or greater, the performance as the protective layer 17 can be sufficiently obtained. It is preferable that the film thickness of the protective layer 17 is 7 nm or less from the viewpoint of reducing the film thickness of the protective layer 17.

As a film forming method of the protective layer 17, a sputtering method using a target material containing carbon, a CVD (chemical vapor deposition) method using a hydrocarbon raw material such as ethylene or toluene, an ion beam deposition (IBD) method, or the like can be used.

In a case where the carbon-based protective layer is formed as the protective layer 17, the carbon-based protective layer can be formed, for example, by a DC magnetron sputtering method. In particular, in a case where a carbon-based protective layer is formed as the protective layer 17, it is preferable to form an amorphous carbon protective layer by a plasma CVD method. The amorphous carbon protective layer formed by the plasma CVD method has a uniform surface and small roughness.

### “Lubricating Layer”

The lubricating layer 18 prevents contamination of the magnetic recording medium 10. In addition, the lubricating layer 18 reduces a frictional force of the magnetic head of the magnetic recording and reproducing device that slides on the magnetic recording medium 10 to improve the durability of the magnetic recording medium 10.

As shown in FIG. 1, the lubricating layer 18 is formed in contact with the protective layer 17. The lubricating layer 18 is formed by applying the lubricant for a magnetic recording medium according to the embodiment described above onto the protective layer 17. Therefore, the lubricating layer 18 contains the above-described fluorine-containing ether compound.

In a case where the protective layer 17 disposed below the lubricating layer 18 is a carbon-based protective layer, the lubricating layer 18 is particularly bonded to the protective layer 17 with a high binding force. As a result, even in a case where the thickness of the lubricating layer 18 is small, the magnetic recording medium 10 in which the surface of the protective layer 17 is coated at a high coating rate is likely to be obtained, and the contamination of the surface of the magnetic recording medium 10 can be effectively prevented.

The average film thickness of the lubricating layer 18 is preferably in a range of 0.5 nm (5 Å) to 2.0 nm (20 Å) and more preferably in a range of 0.5 nm (5 Å) to 1.2 nm (12 Å). In a case where the average film thickness of the lubricating layer 18 is 0.5 nm or greater, the lubricating layer 18 is formed with a uniform film thickness without being island-like or mesh-like. Therefore, the surface of the protective layer 17 can be coated with the lubricating layer 18 at a high coating rate. In addition, in a case where the average film thickness of the lubricating layer 18 is set to 2.0 nm or less, the lubricating layer 18 can be sufficiently thinned, and the floating amount of the magnetic head can be sufficiently reduced.

### “Method for Forming Lubricating Layer”

Examples of a method of forming the lubricating layer 18 include a method of preparing a magnetic recording medium in the middle of production in which each layer up to the protective layer 17 is formed on the substrate 11, coating the protective layer 17 with a lubricating layer forming solution, and drying the solution.

The lubricating layer forming solution is obtained by dispersing and dissolving the lubricant for a magnetic recording medium according to the embodiment described above in a solvent as necessary, and setting the viscosity and the concentration suitable for a coating method.

Examples of a solvent used in the lubricating layer forming solution include a fluorine-based solvent such as VERTREL (registered trademark) XF (trade name, manufactured by DuPont Mitsui Fluorochemicals Co., Ltd.) or ASAHIKLIN (registered trademark) AE-3000 (trade name, manufactured by ACC Inc.).

A coating method for the lubricating layer forming solution is not particularly limited, and examples thereof include a spin coating method, a spraying method, a paper coating method, and a dipping method.

In a case where the dipping method is used, for example, the following method can be used. First, the substrate 11 on which each layer up to the protective layer 17 is formed is immersed in a lubricating layer forming solution placed in an immersion tank of a dip coating device. Next, the substrate 11 is pulled up front the immersion tank at a predetermined speed. In this manner, the lubricating layer forming solution is applied onto the surface of the protective layer 17 of the substrate 11.

By using the dipping method, the lubricating layer forming solution can be uniformly applied to the surface of the protective layer 17, and the lubricating layer 18 can be formed on the protective layer 17 with a uniform film thickness.

In the present embodiment, it is preferable to perform a thermal treatment on the substrate 11 on which the lubricating layer 18 has been formed. By performing the thermal treatment, the adhesion between the lubricating layer 18 and the protective layer 17 is improved, and the adhesion force between the lubricating layer 18 and the protective layer 17 is improved.

The thermal treatment temperature is preferably in a range of 100° C. to 180° C. and more preferably in a range of 100° C. to 160° C. In a case where the thermal treatment temperature is 100° C. or higher, the effect of improving the adhesion between the lubricating layer 18 and the protective layer 17 is sufficiently obtained. In addition, thermal decomposition of the lubricating layer 18 due to the thermal treatment can be prevented by setting the thermal treatment temperature to 180° C. or lower. The thermal treatment time can be appropriately adjusted according to the thermal treatment temperature and is preferably 10 minutes to 120 minutes.

In the present embodiment, in order to further improve the adhesion force of the lubricating layer 18 to the protective layer 17, a treatment of irradiating the lubricating layer 18 with ultraviolet rays (UV) may be performed before or after the thermal treatment.

The magnetic recording medium 10 according to the present embodiment is formed by sequentially providing at least the magnetic layer 16, the protective layer 17, and the lubricating layer 18 on the substrate 11, in the magnetic recording medium 10 according to the present embodiment, the lubricating layer 18 containing the above-described fluorine-containing ether compound is formed in contact with the protective layer 17. The lubricating layer 18 has satisfactory corrosion resistance and is capable of suppressing spin-off even in a case where the film thickness is small. Accordingly, the magnetic recording medium 10 according to the present embodiment ha, excellent reliability, particularly excellent corrosion resistance, spin-off suppression, and durability. Therefore, the magnetic recording medium 10 according to the present embodiment can contribute to the reduction of the magnetic spacing, and the floating amount of the magnetic head can be reduced (for example, 10 nm or less), and the magnetic recording medium 10 can stably operate for a long period of time even in a severe environment due to the diversification of applications. Therefore, the magnetic recording medium 10 according to the present embodiment is particularly suitable as a magnetic disk mounted on a magnetic disk apparatus of a load unload (LUL) system.

### EXAMPLES

Hereinafter, the present invention will be described in more detail with reference to examples and comparative examples. Further, the present invention is not limited to the following examples.

### Example 1

A compound represented by Formula (AA) was obtained by the following method.

A 100 mL eggplant flask was charged with 5 g of a compound represented by HOCH₂CF₂O(CF₂CF₂O)ₕ(CF₂O)ᵢCF₂CH₂OH (h representing the average degree of polymerization in the formula represent 4.5, and i representing the average degree of polymerization represent 4.5) (number-average molecular weight: 1,000, molecular weight distribution: 1.1), 2.70 g of a compound represented by Formula (5-1), and 5 mL, of t-butanol in a nitrogen gas atmosphere, and the mixture was stirred at room temperature until the mixture was uniform. 0.30 g of potassium tert-butoxide was added to the mixture, and the mixture was stirred at 70° C. for 16 hours to carry out a reaction.

The compound represented by Formula (5-1) was synthesized by protecting a 1,2-diol moiety of 1,2,4-butanetriol with acetone and then reacting a hydroxy group at the 4-position with epibromohydrin.

The reaction solution obtained after the reaction was cooled to room temperature, 10 g of a 10% hydrogen chloride/methanol solution (hydrogen chloride-methanol reagent (5-10%) manufactured by Tokyo Chemical Industry Co., Ltd.) was added thereto, and the mixture was stirred at room temperature for 4 hours. Thereafter, the reaction solution was gradually transferred to a separatory funnel containing 25 mL of saturated aqueous sodium bicarbonate, and then extracted twice with 50 mL of ethyl acetate. The organic layer was washed with 25 mL of saline, 25 mL of saturated aqueous sodium bicarbonate, and 25 mL of saline in this order, and then dewatered with anhydrous sodium sulfate. The drying agent was separated by filtration, and the filtrate was concentrated, and the residues were purified by silica gel column chromatography, thereby obtaining 3.71 g of a compound (AA) (Rf₁ in Formula (AA) is a PFPE chain represented by Formula (4-1), and in Rf₁, h representing the average degree of polymerization represents 4.5, and i representing the average degree of polymerization represents 4.5.).

The structure of the obtained compound (AA) was identified by performing ¹H-NMR measurement and ¹⁹F-NMR measurement based on the following results.

¹H-NMR (acetone-D₆): δ [ppm]=1.65-1.85 (4H), 3.40-3.85 (26H), 3.85-4.10 (4H)

¹⁹F-NMR (acetone-D₆): δ [ppm]=−55.5 to −51.5 (9F), −78.5 (2F), −80.5 (2F). −91.0 to −88.5 (18F)

### Example 2

A compound represented by Formula (AB) was obtained by the following method.

3.85 g of a compound (AB) (Rf₁ in Formula (AB) is a PFPE chain represented by Formula (4-1), and in Rf₁, h representing the average degree of polymerization represents 4.5, and i representing the average degree of polymerization represents 4.5) was obtained by performing the same operation as in Example 1 except that a compound represented by Formula (5-2) was used instead of the compound represented by Formula (5-1).

The compound represented by Formula (5.2) was synthesized by protecting a 1,2-diol moiety of 1,2,6-hexanetriol with acetone and then reacting a hydroxy group at the 6-position with epibromohydrin.

The structure of the obtained compound (AB) was identified by performing ¹H-NMR measurement and ¹⁹F-NMR measurement based on the following results.

¹H-NMR (acetone-D₆): δ [ppm]=1.65-1.85 (12H), 3.40-3.85 (26H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 3

A compound represented by Formula (AC) was obtained by the following method.

3.85 g of a compound (AC) (Rf₁: in Formula (AC) is a PFPE chain represented by Formula (4-1), and in Rf₁, h representing the average degree of polymerization represents 4.5, and i representing the average degree of polymerization represents 4.5) was obtained by performing the same operation as in Example 1 except that a compound represented by Formula (5-3) was used instead of the compound represented by Formula (5-1).

The compound represented by Formula (5-3) was synthesized by protecting a 1,2-diol moiety of 1,2,8-octantriol with acetone and then reacting a hydroxy group at the 8-position with epibromohydrin.

The structure of the obtained compound (AC) was identified by performing ¹H-NMR measurement and ¹⁹F-NMR measurement based on the following results. ¹H-NMR (acetone-D₆): δ [ppm]=1.65-1.85 (20H), 3.40-3.85 (26H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 41

A compound represented by Formula (AD) was obtained by the following method.

3.91 g of a compound (AD) (Rf₁ in Formula (AD) is a PFPE chain represented by Formula (4-1), and in Rf₁, h representing the average degree of polymerization represents 4.5, and i representing the average degree of polymerization represents 4.5) was obtained by performing the same operation as in Example 1 except that a compound represented by Formula (5-4) was used instead of the compound represented by Formula (5-1).

The compound represented by Formula (5-4) was synthesized by the following method. The 1,2-diol moiety of 1,2,4-butanetriol was protected with acetone, and the hydroxy group at the 4-position was brominated and reacted with 3-butene-1-ol, thereby obtaining a compound. The vinyl group of the obtained compound was oxidized using m-chloroperbenzoic acid (mCPBA) to synthesize the compound.

The structure of the obtained compound (AD) was identified by performing ¹H-NMR measurement and ¹⁹F-NMR measurement based on the following results.

¹H-NMR (acetone-D₆): δ [ppm]=1.65-1.85 (8H), 3.40-3.85 (26H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 5

A compound represented by Formula (AE) was obtained by the following method.

3.26 g of a compound (AE) (Rf₁ in Formula (AE) is a PFPE, chain represented by Formula (4-1), and in Rf), h representing the average degree of polymerization represents 4.5, and i representing the average degree of polymerization represents 4.5) was obtained by performing the same operation as in Example 1 except that a compound represented by Formula (5-5) was used instead of the compound represented by Formula (5-1).

The compound represented by Formula (5-5) was synthesized by reacting the compound obtained by brominating the hydroxy group of solketal (2,2-dimethyl-1,3-dioxolane-4-methanol) with 3-butene-1-ol and oxidizing the vinyl group of the obtained compound using m-chloroperbenzoic acid (mCPBA).

The structure of the obtained compound (AE) was identified by performing ¹H-NMR measurement and ¹⁹F-NMR measurement based on the following results.

¹H-NMR (acetone-D₆): δ [ppm]=1.65-1.85 (4H), 3.40-3.85 (26H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 61

A compound represented by Formula (AF) was obtained by the following method.

3.43 g of a compound (AF) (Rf₁ in Formula (AF) is a PFPE chain represented by Formula (4-1), and in Rf₁ h representing the average degree of polymerization represents 4.5, and i representing the average degree of polymerization represents 4.5) was obtained by performing the same operation as in Example 1 except that a compound represented by Formula (5-6) was used instead of the compound represented by Formula (5-4).

The compound represented by Formula (5-6) was synthesized by reacting the compound obtained by brominating the hydroxy group of solketal with 5-hexene-1-ol and oxidizing the vinyl group of the obtained compound using m-chloroperbenzoic acid (mCPBA).

The structure of the obtained compound (AF) was identified by performing ¹H-NMR measurement and ¹⁹F-NMR measurement based on the following results.

¹H-NMR (acetone-D₆): δ [ppm]=1.65-1.85 (121H), 3.40-3.85 (26H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 71

A compound represented by Formula (AG) was obtained by the following method.

3.56 g of a compound (AG) (Rf₁ in Formula (AG) is a PFPE chain represented by Formula (4-1), and in Rf₁, h representing the average degree of polymerization represents 4.5, and i representing the average degree of polymerization represents 4.5) was obtained by performing the same operation as in Example 1 except that a compound represented by Formula (5-7) was used instead of the compound represented by Formula (5-1).

The compound represented by Formula (5-7) was synthesized by reacting the compound obtained by brominating the hydroxy group of solketal with 7-octene-1-ol and oxidizing the vinyl group of the obtained compound using m-chloroperbenzoic acid (mCPBA).

The structure of the obtained compound (AG) was identified by performing ¹H-NMR measurement and ¹⁹F-NMR measurement based on the following results.

¹H-NMR (acetone-D₆): δ [ppm]=1.65-1.85 (20H), 3.40-3.85 (26H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 8

A compound represented by Formula (AH) was obtained by the following method.

3.40 g of a compound (AH) (Rf₁ in Formula (AH) is a PFPE chain represented by Formula (4-1), and in Rf₁ h representing the average degree of polymerization represents 4.5, and i representing the average degree of polymerization represents 4.5) was obtained by performing the same operation as in Example 1 except that a compound represented by Formula (5-8) was used instead of the compound represented by Formula (5-1).

The compound represented by Formula (5-8) was synthesized by the following method. An epoxy group of 1,2-epoxy-5-hexene was ring-opened using dilute sulfuric acid. The compound was synthesized by protecting the 1,2-diol moiety generated by the ring opening using acetone and oxidizing the vinyl group of the obtained compound using m-chloroperbenzoic acid (mCPBA).

The structure of the obtained compound (AH) was identified by performing ¹H-NMR measurement and ¹⁹F-NMR measurement based on the following results.

¹H-NMR (acetone-D₆): δ [ppm]=1.65-1.85 (8H), 3.40-3.85 (18H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 9

A compound represented by Formula (AI) was obtained by the following method.

3.45 g of a compound (AI) (Rf₁ in Formula (AI) is a PFPE chain represented by Formula (4-1), and in Rf₁, h representing the average degree of polymerization represents 4.5, and i representing the average degree of polymerization represents 4.5) was obtained by performing the same operation as in Example 1 except that a compound represented by Formula (5-9) was used instead of the compound represented by Formula (5-1).

The compound represented by Formula (5-9) was synthesized by the following method. One vinyl group of 1,7-octadiene was oxidized using m-chloroperbenzoic acid (mCPBA). The epoxy group generated by oxidation was ring-opened using dilute sulfuric acid. The compound was synthesized by protecting the 1,2-diol moiety generated by the ring opening using acetone and oxidizing the vinyl group of the obtained compound using mCPBA.

The structure of the obtained compound (AI) was identified by performing ¹H-NMR measurement and ¹⁹F-NMR measurement based on the following results.

¹H-NMR (acetone-D₆): δ [ppm]=1.65-1.85 (16H), 3.40-3.85 (18H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 10

A compound represented by Formula A) was obtained by the following method.

3.68 g of a compound (AJ) (Rf₁ in Formula (AJ) is a PFPE chain represented by Formula (4-1), and in Rf₁, h representing the average degree of polymerization represents 4.5, and i representing the average degree of polymerization represents 4.5) was obtained by performing the same operation as in Example 1 except that a compound represented by Formula (5-10) was used instead of the compound represented by Formula (5-1).

The compound represented by Formula (5.10) was synthesized by reacting one hydroxy group of 1,3-propanediol with the compound obtained by brominating the hydroxy group of solketal and reacting the other hydroxy group with epibromohydrin.

The structure of the obtained compound (AJ) was identified by performing ¹H-NMR measurement and ¹⁹F-NMR measurement based on the following results.

¹H-NMR (acetone-D₆): δ [ppm]=1.65-1.85 (4H), 3.40-3.85 (34H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 11

A compound represented by Formula (AK) was obtained by the following method.

3.76 g of a compound (AK) (Rf₁ in Formula (AK) is a PFPE chain represented by Formula (4-1), and in Rf₁, h representing the average degree of polymerization represents 4.5, and i representing the average degree of polymerization represents 4.5) was obtained by performing the same operation as in Example 1 except that a compound represented by Formula (5-11) was used instead of the compound represented by Formula (5-1).

The compound represented by Formula (5-11) was synthesized by reacting one hydroxy group of 2,2-dimethyl-1,3-propanediol with the compound obtained by brominating the hydroxy group of solketal and reacting the other hydroxy group with epibromohydrin.

The structure of the obtained compound (AK) was identified by performing ¹H-NMR measurement and ¹⁹F-NMR measurement based on the following results.

¹H-NMR (acetone-D₆): δ [ppm]=1.20-1.25 (12H), 3.40-3.85 (34H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 121

A compound represented by Formula (AL) was obtained by the following method.

3.78 g of a compound (AL) (Rf₁ in Formula (AL) is a PFPE chain represented by Formula (4-1), and in Rf₁, h representing the average degree of polymerization represents 4.5, and i representing the average degree of polymerization represents 4.5) was obtained by performing the same operation as in Example 1 except that a compound represented by Formula (5-12) was used instead of the compound represented by Formula (5-1).

The compound represented by Formula (5-12) was synthesized by reacting one hydroxy group of 1,4-butanediol with the compound obtained by brominating the hydroxy group of solketal and reacting the other hydroxy group with epibromohydrin.

The structure of the obtained compound (AL) was identified by performing ¹H-NMR measurement and ¹⁹F-NMR measurement based on the following results.

¹H-NMR (acetone-D₆): δ [ppm]=1.65-1.85 (8H), 3.40-3.85 (34H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 13

A compound represented by Formula (AM) was obtained by the following method.

3.85 g of a compound (AM) (Rf₁ in Formula (AM) is a PFPE chain represented by Formula (4-1), and in Rf₁, h representing the average degree of polymerization represents 4.5, and i representing the average degree of polymerization represents 4.5) was obtained by performing the same operation as in Example 1 except that a compound represented by Formula (5-13) was used instead of the compound represented by Formula (5-1).

The compound represented by Formula (5-13) was synthesized by reacting one hydroxy group of 2,3-dimethyl-1,4-butanediol with the compound obtained by brominating the hydroxy group of solketal and reacting the other hydroxy group with epibromohydrin.

The structure of the obtained compound (AM) was identified by performing ¹H-NMR measurement and ¹⁹F-NMR measurement based on the following results. ¹H-NMR (acetone-16): δ [ppm]=1.20-1.25 (12H), 1.65-1.85 (4H), 3.40-3.85 (34H), 3.85-4.10 (41H)

¹⁹F-NMR (acetone-D₆): δ [ppm]=−55.5 to −51.5 (9F), −78.5 (2F), −80.5 (2F), −91.0 to −88.5 (18F)

### Example 14

A compound represented by Formula (AN) was obtained by the following method.

3.81 g of a compound (AN) (Rf₁ in Formula (AN) is a PFPE chain represented by Formula (4-1), and in Rf₁, h representing the average degree of polymerization represents 4.5, and i representing the average degree of polymerization represents 4.5) was obtained by performing the same operation as in Example 1 except that a compound represented by Formula (5-14) was used instead of the compound represented by Formula (5-1.

The compound represented by Formula (5-14) was synthesized by reacting one hydroxy group of 1,8-octanediol with the compound obtained by brominating the hydroxy group of solketal and reacting the other hydroxy group with epibromohydrin.

The structure of the obtained compound (AN) was identified by performing ¹H-NMR measurement and ¹⁹F-NMR measurement based on the following results.

¹H-NMR (acetone-D₆): δ [ppm]=1.65-1.85 (24H), 3.40-3.85 (34H), 3.85-4.10 (4H)

¹⁹F-NMR (acetone-D₆): δ [ppm]=−55.5 to −51.5 (9F), −78.5 (2F), −80.5 (2F). −91.0 to −88.5 (18F)

### Example 15

A compound represented by Formula (AO) was obtained by the following method.

4.02 g of a compound (AO) (Rf₁ in Formula (AO) is a PFPE chain represented by Formula (4-1), and in Rf₁, h representing the average degree of polymerization represents 4.5, and i representing the average degree of polymerization represents 4.5) was obtained by performing the same operation as in Example 1 except that a compound represented by Formula (5-15) was used instead of the compound represented by Formula (5-1).

The compound represented by Formula (5-15) was synthesized by reacting one hydroxy group of 2,2,3,3-tetrafluoro-1,4-butanediol with the compound obtained by brominating the hydroxy group of solketal and reacting the other hydroxy group with epibromohydrin.

The structure of the obtained compound (AO) was identified by performing ¹H-NMR measurement and ¹⁹F-NMR measurement based on the following results.

¹H-NMR (acetone-D₆): δ [ppm]=3.40-3.85 (26H), 3.85-4.10 (12H)

¹⁹F-NMR (acetone-D₆): δ [ppm]=−55.5 to −51.5 (9F), −78.5 (2F), −80.5 (2F), −91.0 to −88.5 (18F), −128.5 to 130.0 (8F)

### Example 16

A compound represented by Formula (AP) was obtained by the following method.

4.31 g of a compound (AP) (Rf₁ in Formula (AP) is a PFPE chain represented by Formula (4-1), and in Rf₁, h representing the average degree of polymerization represents 4.5, and i representing the average degree of polymerization represents 4.5) was obtained by performing the same operation as in Example 1 except that a compound represented by Formula (5-16) was used instead of the compound represented by Formula (5-1).

The compound represented by Formula (5-16) was synthesized by reacting one hydroxy group of 2,2,3,3,4,4,5,5,6,6,7,7-dodecafluoro-1,8-octanediol with the compound obtained by brominating the hydroxy group of solketal and reacting the other hydroxy group with epibromohydrin.

The structure of the obtained compound (AP) was identified by performing ¹H-NMR measurement and ¹⁹F-NMR measurement based on the following results.

¹H-NMR (acetone-D₆): δ [ppm]=3.40-3.85 (26H), 3.85-4.10 (12H)

¹⁹F-NMR (acetone-D₆): δ [ppm]=−55.5 to −51.5 (9F), −78.5 (2F), −80.5 (2F), −91.0 to −88.5 (18F), −122.5 to −124.5 (16F), −128.5 to 130.0 (8F)

### Example 17

A compound represented by Formula (AQ) was obtained by the following method.

A 100 mL eggplant flask was charged with 5 g of a compound represented by HOCH₂CF₂O(CF₂CF₂O)ₕ(CF₂O)ᵢ(CF₂CH₂OH (h representing the average degree of polymerization in the formula represents 4.5, and i representing the average degree of polymerization represents 4.5) (number-average molecular weight: 1,000, molecular weight distribution: 1.1), 4.50 g of 1-bromo-4-pentene, and 10 mL, of N,N-dimethylformamide in a nitrogen gas atmosphere, and the mixture was stirred at room temperature until the mixture was uniform. 1.20 g of sodium hydride (purity: 60%, containing mineral oil) was added to the mixture, and the mixture was stirred at 70° C. for 16 hours to carry out a reaction.

The reaction product obtained after the reaction was cooled to 25° C., transferred to a separatory funnel charged with 50 mL of water, and extracted three times with 100 mL of ethyl acetate. The organic layer was washed with water and dewatered with anhydrous sodium sulfate. The drying agent, was separated by filtration, and the filtrate was concentrated, and the residues were purified by silica gel column chromatography, thereby obtaining 4.76 g of a compound represented by Formula (7-1) as an intermediate compound.

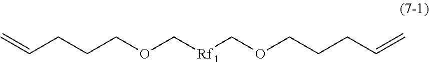

(Rf₁ in Formula (7-1) is a PFPE chain represented by Formula (4-1). In Rf₁, h representing the average degree of polymerization represents 4.5, and i representing the average degree of polymerization represents 4.5.)

Subsequently, a 100 mL eggplant flask was charged with 4.76 g of the compound represented by Formula (7-1) as an intermediate compound obtained above, 20 mL of methylene chloride, and 3.15 g of m-chloroperbenzoic acid (nCPBA (purity: 73%, containing water)) in a nitrogen gas atmosphere, and the mixture was stirred at room temperature for 16 hours to carry out a reaction.

50 mL of an aqueous solution of 3.18 g of sodium sulfite was added to the reaction solution obtained after the reaction to deactivate excess mCPBA. The generated solid was separated by filtration, transferred to a separatory funnel, and extracted three times with 100 mL of methylene chloride. The organic layer was washed with water and dewatered with anhydrous sodium sulfate. The drying agent was separated by filtration, and the filtrate was concentrated, and the residues were purified by silica gel column chromatography, thereby obtaining 4.21 g of a compound represented by Formula (7-2) as an intermediate compound.

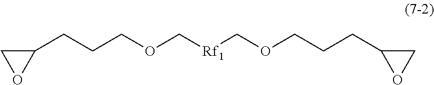

(Rf₁ in Formula (7-2) is a PPE chain represented by Formula (4-1). In Rf₁, h representing the average degree of polymerization represents 4.5, and i representing the average degree of polymerization represent 4.5.)

Subsequently, a 100 mL eggplant flask was charged with 4.21 g of the compound represented by Formula (7-2) as an intermediate compound obtained above, 20 mL of acetone, and 20 ml of 10% dilute sulfuric acid in a nitrogen gas atmosphere, and the mixture was stirred at room temperature for 16 hours to carry out a reaction.

20 mL of saturated aqueous sodium bicarbonate was added to the reaction solution obtained after the reaction to neutralize the reaction solution, and the generated solid was separated by filtration. Thereafter, the mixture was transferred to a separatory funnel and extracted three times with 100 mL of ethyl acetate. The organic layer was washed with water and dewatered with anhydrous sodium sulfate. The drying agent was separated by filtration, the filtrate was concentrated, and the residues were purified by silica gel column chromatography, thereby obtaining 3.32 g of a compound (AQ) Rf₁ in Formula (AQ) is a PFPE chain represented by Formula (4-1), and in Rf₁, h representing the average degree of polymerization represents 4.5, and i representing the average degree of polymerization represents 4.5).

The structure of the obtained compound (AQ) was identified by performing ¹H-NMR measurement and ¹⁹F-NMR measurement based on the following results.

¹H-NMR (acetone-D₆): δ [ppm]=1.65-1.85 (8H), 3.40-3.85 (14H), 3.85-4.10 (4H)

¹⁹F-NMR (acetone-D₆): δ [ppm]=−55.5 to −51.5 (9F), −78.5 (21F), −80.5 (2F), −91.0 to −88.5 (18F)

### Example 18

A compound represented by Formula (AR) was obtained by the following method.

3.35 g of a compound (AR) (Rf₁ in Formula (AR) is a PFPE chain represented by Formula (4-1), and in Rf₁, h representing the average degree of polymerization represents 4.5, and i representing the average degree of polymerization represents 4.5) was obtained by performing the same operation as in Example 17 except that 1-bromo-7-octene was used instead of 1-bromo-4-pentene.

The structure of the obtained compound (AR) was identified by performing ¹H-NMR measurement and ¹⁹F-NMR measurement, based on the following results.

¹H-NMR (acetone-Deo: δ [ppm]=1.65-1.85 (20H), 3.40-3.85 (14H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 19

A compound represented by Formula (AS) was obtained by the following method.

### First Reaction

A 100 mL eggplant flask was charged with 12.5 g of a compound represented by HOCH₂CF₂O(CF₂CF₂O)ₕ(CF₂O)ᵢCF₂CH₂OH (h representing the average degree of polymerization in the formula represents 4.5, and i representing the average degree of polymerization represents 4.5) (number-average molecular weight: 1,000, molecular weight distribution: 1.1), 2.70 g of the compound represented by Formula (5-1), and 12 mL of t-butanol in a nitrogen gas atmosphere, and the mixture was stirred at room temperature until the mixture was uniform. 1.10 g of potassium tert-butoxide was added to the mixture, and the mixture was stirred at 70° C. for 16 hours to carry out a reaction.

The reaction product obtained after the reaction was cooled to 25° C., transferred to a separatory funnel charged with 100 mL of water, and extracted three times with 100 mL of ethyl acetate. The organic layer was washed with water and dewatered with anhydrous sodium sulfate. The drying agent was separated by filtration, and the filtrate was concentrated, and the residues was purified by silica gel column chromatography, thereby obtaining 7.21 g of a compound represented by in Formula (8) as an intermediate compound 1.

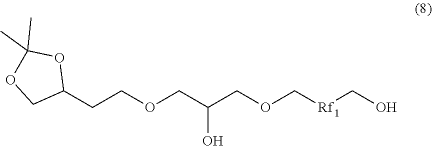

(Rf₁ in Formula (8) is a PFPE chain represented by Formula (4-1). In Rf₃, h representing the average degree of polymerization represents 4.5, and i representing the average degree of polymerization represents 4.5.)

### Second Reaction

Subsequently, a 100 mL eggplant flask was charged with 7.21 g of the compound represented by Formula (8) as an intermediate compound 1 obtained above, 2.70 g of the compound represented by Formula (5-5), and 20 mL of t-butanol in a nitrogen gas atmosphere, and the mixture was stirred at room temperature until the mixture is uniform. 0.55 g of potassium tert-butoxide was added to the mixture, and the mixture was stirred at 70° C. for 16 hours to carry out a reaction.

The reaction solution obtained after the reaction was cooled to room temperature, 50 g of a 10% hydrogen chloride/methanol solution (hydrogen chloride-methanol reagent (5-10%), manufactured by Tokyo Chemical Industry Co., Ltd.) was added thereto, and the mixture was stirred at room temperature for 4 hours. Thereafter, the reaction solution was gradually transferred to a separatory funnel containing 100 mL of saturated aqueous sodium bicarbonate, and then extracted twice with 200 mL of ethyl acetate. The organic layer was washed with 100 mL of saline, 100 mL of saturated aqueous sodium bicarbonate, and 100 mL of saline in this order, and then dewatered with anhydrous sodium sulfate. The drying agent was separated by filtration, and the filtrate was concentrated, and the residues were purified by silica gel column chromatography, thereby obtaining 3.44 g of a compound (AS) (Rf₁ in Formula (AS) is a PFPE chain represented by Formula (4-1), and in Rf₁, h representing the average degree of polymerization represents 4.5, and i representing the average degree of polymerization represents 4.5.).

The structure of the obtained compound (AS) was identified by preforming ¹H-NMR measurement and ¹⁹F-NMR measurement based on the following results.

¹H-NMR (acetone-D₆): δ [ppm]=1.65-1.85 (4H), 3.40-3.85 (26H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 20

A compound represented by Formula (BA) was obtained by the following method.

3.61 g of a compound (BA) (Rf₁: in Formula (BA) is a PFPE chain represented by Formula (4-1), and in Rf₁, h representing the average degree of polymerization represents 4.5, and i representing the average degree of polymerization represents 4.5) was obtained by performing the same operation as in Example 19 except that a compound represented by Formula (5-17) was used instead of the compound represented by Formula (5-5).

The compound represented by Formula (5-17) was synthesized by protecting a hydroxy group of ethylene glycol monoallyl ether using dihydropyran and then oxidizing a vinyl group with m-chloroperbenzoic acid (mCPBA).

The structure of the obtained compound (BA) was identified by performing ¹H-NMR measurement and ¹⁹F-NMR measurement based on the following results.

¹H-NMR (acetone-D₆): δ [ppm]=1.65-1.85 (2H), 3.40-3.85 (24H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 21

A compound represented by Formula (BB) was obtained by the following method.

3.61 g of a compound (BB) (Rf₁ in Formula (BB) is a PFPE chain represented by Formula (4-1), and in Rf₁, h representing the average degree of polymerization represents 4.5, and i representing the average degree of polymerization represents 4.5) was obtained by performing the same operation as in Example 19 except that a compound represented by Formula (5-18) was used instead of the compound represented by Formula (5-5).

The compound represented by Formula (5-18) was synthesized by the following method. Epichlorohydrin reacted with 3-butene-1-ol in an amount twice the molar amount of the epichlorohydrin. A hydroxy group generated by the reaction was protected with dihydropyran. One vinyl group of the compound obtained by the reaction was synthesized by oxidation with m-chloroperbenzoic acid (mCPBA).

The structure of the obtained compound (BB) was identified by performing ¹H-NMR measurement and ¹⁹F-NMR measurement based on the following results.

¹H-NMR (acetone-D₆): δ [ppm]=1.65-1.85 (4H), 3.40-3.85 (27H), 3.85-4.10 (4H), 5.20-5.80 (3H)

¹⁹F-NMR (acetone-D₆): δ [ppm]=−55.5 to −51.5 (9F) −78.5 (2F), −80.5 (2F), −91.0 to −88.5 (18F)

### Example 22

A compound represented by Formula (BC) was obtained by the following method.

3.53 g of a compound (BC) (Rf₁ in Formula (BC) is a PEPE chain represented by Formula (4-1), and in Rf), h representing the average degree of polymerization represents 4.5, and i representing the average degree of polymerization represents 4.5) was obtained by performing the same operation as in Example 19 except that a compound represented by Formula (5-19) was used instead of the compound represented by Formula (5-5).

The compound represented by Formula (5-19) was synthesized by reacting a hydroxy group of 2-acetoamidoethanol with epibromohydrin.

The structure of the obtained compound (BC) was identified by performing ¹H-NMR measurement and ¹⁹F-NMR measurement, based on the following results.

¹H-NMR (acetone-D₆): δ [ppm]=1.65-1.85 (2H), 1.90 (3H), 3.40-3.85 (23H) 3.85-4.10 (4H), 6.70-6.80 (1H)

¹⁹F-NMR (acetone-D₆): δ [ppm]=−55.5 to −51.5 (9F), −78.5 (2F), −80.5 (2F), −91.0 to −88.5 (18F)

### Example 23

A compound represented by Formula (BD) was obtained by the following method.

3.72 g of a compound (BD) (Rf₁ in Formula (BD) is a PFPE chain represented by Formula (4-1), and in Rf₁, h representing the average degree of polymerization represents 4.5, and i representing the average degree of polymerization represents 4.5) was obtained by performing the same operation as in Example 19 except that a compound represented by Formula (5-20) was used instead of the compound represented by Formula (5-5).

The compound represented by Formula (5-20) was synthesized by reacting a hydroxy group of 3-cyanopropanol with epibromohydrin.

The structure of the obtained compound (BD) was identified by performing ¹H-NMR measurement and ¹⁹F-NMR measurement based on the following results.

¹H-NMR (acetone-D₆): δ [ppm]=1.65-1.85 (4H), 1.90-2.00 (2H), 3.40-3.85 (21H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 24

A compound represented by Formula (BE) was obtained by the following method.

3.72 g of a compound (BE) (Rf₁ in Formula (BE) is a PFPE chain represented by Formula (4-1), and in Rf₁, h representing the average degree of polymerization represents 4.5, and i representing the average degree of polymerization represents 4.5) was obtained by performing the same operation as in Example 19 except that a compound represented by Formula (5-21) was used instead of the compound represented by Formula (5-5).

The compound represented by Formula (5-21) was synthesized by protecting a hydroxy group of 3-butene-1-ol using dihydropyran and then oxidizing a vinyl group with m-chloroperbenzoic acid (mCPBA).

The structure of the obtained compound (BE) was identified by performing ¹H-NMR measurement and ¹⁹F-NMR measurement based on the following results.

¹H-NMR (acetone-D₆): δ [ppm]=1.65-1.85 (4H), 3.40-3.85 (20H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 25

A compound represented by Formula (BF) was obtained by the following method.

3.72 g of a compound (BF) (Rf₁ in Formula (BF) is a PFPE chain represented by Formula (4-1), and in Rf₁, h representing the average degree of polymerization represents 4.5, and i representing the average degree of polymerization represents 4.5) was obtained by performing the same operation as in Example 19 except that a compound represented by Formula (5-22) was used instead of the compound represented by Formula (5-5).

The compound represented by Formula (5-22) was synthesized by protecting two hydroxy groups of 3-allyloxy-1,2-propanediol with dihydropyran and then oxidizing a vinyl group with m-chloroperbenzoic acid (nCPBA).

The structure of the obtained compound (BF) was identified by performing ¹H-NMR measurement and ¹⁹F-NMR measurement based on the following results.

¹H-NMR (acetone-D₆): δ [ppm]=1.65-1.85 (2H), 3.40-3.85 (26H), 3.85-4.10 (4H)

¹⁹F-NMR (acetone-Mk): δ [ppm]=−55.5 to −51.5 (9F), −78.5 (2F), −80.5 (2F), −91.0 to −88.5 (18F)

### Example 26

A compound represented by Formula (BG) was obtained by the following method.

3.48 g of a compound (BG) (Rf₂ in Formula (BG) is a PFPE chain represented by Formula (4-2), and in Rf₁, j representing the average degree of polymerization represents 4.5) was obtained by performing the same operation as in Example 1 except that a compound represented by HOCH₂CF₂CF₂O(CF₂CF₂CF₂O)ⱼCF₂CF₂CH₂OH (j representing the average degree of polymerization in the formula represents 4.5) (number-average molecular weight: 1,000, molecular weight distribution: 1.1) was used instead of the compound represented by HOCH₂CF₂CH₂O(CF₂CF₂O)ₕ(CF₂O)ⱼCF₂CH₂OH.

The structure of the obtained compound (BG) was identified by performing ¹H-NMR measurement and ¹⁹F-NMR measurement based on the following results.

¹H-NMR (acetone-D₆): δ [ppm]=1.65-1.85 (4H), 3.40-3.85 (26H), 3.85-4.10 (4H)

¹⁹F-NMR (acetone-D₆): δ [ppm]=−84.0 to −83.0 (18F), −86.4 (41F), −124.3 (4F), −130.0 to −129.0 (9F)

### Example 27

A compound represented by Formula (BH) was obtained by the following method.

3.44 g of a compound (BG) (Rf₃ in Formula (BH) is a PFPE chain represented by Formula (4-3), and in Rf₃, k representing the average degree of polymerization represents 3.0) was obtained by performing the same operation as in Example 1 except that a compound represented by HOCH₂CF₂CF₂CF₂O(CF₂CF₂CF₂CF₂O)ₖCF₂CF₂CF₂CH₂OH (k representing the average degree of polymerization in the formula represents 3.0) (number-average molecular weight: 1,000, molecular weight distribution: 1.1) was used instead of the compound represented by HOCH₂CF₂O(CF₂CF₂O)ₕ(CF₂O)ⱼCF₂CH₂OH.

The structure of the obtained compound (BH) was identified by performing ¹H-NMR measurement and ¹⁹F-NMR measurement based on the following results.

¹H-NMR (acetone-D₆): δ [ppm]=1.65-1.85 (4H), 3.40-3.85 (26H), 3.85-4.10 (4H)

¹⁹F-NMR (acetone-D₆): δ [ppm]=−84.0 to −83.0 (16F), −122.5 (4F), −126.0 (12F), −129.0 to −128.0 (4F)

### Example 28

A compound represented by Formula (CA) was obtained by the following method.

3.15 g of a compound (CA) (Rf₁ in Formula (CA) is a PFPE chain represented by Formula (4-1), and in Rf₁, h representing the average degree of polymerization represents 7.0 and i representing the average degree of polymerization represents 0) was obtained by performing the same operation as in Example 1 except that a compound represented by HOCH₂CF₂O(CF₂CF₂O)ₕ(CF₂O)CF₂CH₂OH (h representing the average degree of polymerization in the formula represents 7.0 and i representing the average degree of polymerization represents 0) was used instead of the compound represented by HOCH₂CF₂O(CF₂CF₂O)ₙ(CF₂O)ᵢCF₂CH₂OH (h representing the average degree of polymerization in the formula represents 4.5 and i representing the average degree of polymerization represents 4.5).

The structure of the obtained compound (CA) was identified by performing ¹H-NMR measurement and ¹⁹F-NMR measurement based on the following results.

¹H-NMR (acetone-D₆): δ [ppm]=1.65-1.85 (4H), 3.40-3.85 (26H), 3.85-4.10 (4H)

¹⁹F-NMR (acetone-D₆): δ [ppm]=−78.5 (4F), −91.0 to −88.5 (28F)

### Example 29

A compound represented by Formula (CB) was obtained by the following method.

3.08 g of a compound (CB) (Rf₁ in Formula (CB) is a PFPE chain represented by Formula (4-1), and in Rf₁, h representing the average degree of polymerization represents 7.0, and i representing the average degree of polymerization represents 0) was obtained by performing the same operation as in Example 28 except that a compound represented by Formula (5-5) was used instead of the compound represented by Formula (5-1).

The structure of the obtained compound (CB) was identified by performing ¹H-NMR measurement and ¹⁹F-NMR measurement based on the following results.

¹H-NMR (acetone-D₆: δ [ppm]=1.65-1.85 (4H), 3.40-3.85 (26H), 3.85-4.10 (4H)

¹⁹F-NMR (acetone-D₆): δ [ppm]=−78.5 (4F), −91.0 to −88.5 (28F)

### Example 30

A compound represented by Formula (CC) was obtained by the following method.

3.24 g of a compound (CC) (Rf₂ in Formula (CC) is a PFPE chain represented by Formula (4-2), and in Rf₂, j representing the average degree of polymerization represents 4.5) was obtained by performing the same operation as in Example 26 except that a compound represented by Formula (5-5) was used instead of the compound represented by Formula (5-1).

The structure of the obtained compound (CC) was identified by performing ¹H-NMR measurement and ¹⁹F-NMR measurement based on the following results.

¹H-NMR (acetone-D₆): δ [ppm]=1.65-1.85 (4H), 3.40-3.85 (26H), 3.85-4.10 (4H)

¹⁹F-NMR (acetone-D₆): δ [ppm]=−84.0 to −83.0 (18F), −86.4 (4F), −124.3 (4F), −130.0 to −129.0 (9F)

### Example 31

A compound represented by Formula (CD) was obtained by the following method.

2.95 g of a compound (CD) (Rf₃ in Formula (CD) is a PFPE chain represented by Formula (4-3), and in Rf₃, k representing the average degree of polymerization represents 3.0) was obtained by performing the same operation as in Example 27 except that a compound represented by Formula (5-5) was used instead of the compound represented by Formula (5-1).

The structure of the obtained compound (CD) was identified by performing ¹H-NMR measurement and ¹⁹F-NMR measurement based on the following results.

¹H-NMR (acetone-D₆): δ [ppm]=1.65-1.85 (4H), 3.40-3.85 (26H), 3.85-4.10 (4H)

¹⁹F-NMR (acetone-D₆): δ [ppm]=−84.0 to −83.0 (6F), −122.5 (4F), −126.0 (12F), −129.0 to −128.0 (4F)

### Example 32

A compound represented by Formula (CE) was obtained by the following method.

3.35 g of a compound (CE) (Rf₁ in Formula (CE) is a PFPE chain represented by Formula (4-1), and in Rf₁, h representing the average degree of polymerization represents 4.5 and i representing the average degree of polymerization represents 4.5) was obtained by performing the same operation as in Example 1 except that a compound represented by Formula (5-24) was used instead of the compound represented by Formula (5-1). The compound represented by Formula (5-24) was synthesized by reacting one hydroxy group of 2-methyl-1,3-propanediol with the compound obtained by brominating the hydroxy group of solketal and reacting the other hydroxy group with epibromohydrin.

The structure of the obtained compound (CE) was identified by performing ¹H-NMR measurement and ¹⁹F-NMR measurement based on the following results.

¹H-NMR (acetone-D₆): δ [ppm]=1.20-1.25 (6H), 1.25-1.35 (2H), 3.40-3.85 (34H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 33

A compound represented by Formula (CF) was obtained by the following method.

A 100 mL eggplant flask was charged with 5 g of a compound represented by HOCH₂CF₂CF₂(CF₂CF₂CF₂O)ⱼCF₂CF₂CH₂OH (j representing the average degree of polymerization in the formula represents 4.5) (number-average molecular weight: 1,000, molecular weight distribution: 1.1), 4.83 g of the compound represented by Formula (5-25), and 20 mL of N,N-dimethylformamide in a nitrogen gas atmosphere, and the mixture was stirred at room temperature until the mixture was uniform. 7.14 g of cesium carbonate was added to the mixture, and the mixture was stirred at 70° C. for 16 hours to carry out a reaction.

The compound represented by Formula (5-25) was synthesized by the following method. First, a hydroxy group of 3-butene-1-ol was protected using dihydropyran, and then an alkenyl group was oxidized with in-chloroperbenzoic acid. Thereafter, the obtained compound reacted with solketal, and the secondary hydroxy group of the generated compound was protected using chloromethyl methyl ether. The obtained compound represented by Formula (5-25A) was treated with an acid to selectively deprotect the THP group, and a primary hydroxy group reacted with p-toluenesulfonyl chloride, thereby obtaining a compound represented by Formula (5-25).

The selective deprotection of the THP group was carried out by adding 0.52 g (2.07 nmol) of p-toluenesulfonic acid pyridinium serving as an acid catalyst to a mixture obtained by mixing 3.60 g (10.3 mmol) of the compound represented by Formula (5-25A) and a mixed solvent formed by mixing 16 g of 2-propanol and 16 g of acetone, and stirring the mixture at a reaction temperature of 55° C. for 7 hours in an air atmosphere.

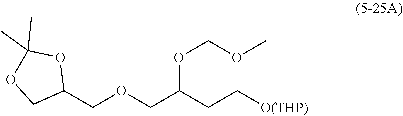

The reaction solution obtained after the reaction of the fluorine-based compound with the compound represented by Formula (5-25) was cooled to room temperature, 10 g of a 10% hydrogen chloride/methanol solution (hydrogen chloride-methanol reagent (5-10%), manufactured by Tokyo Chemical Industry Co., Ltd.) was added thereto, and the mixture was stirred at room temperature for 4 hours. Thereafter, the reaction solution was gradually transferred to a separatory funnel containing 25 mL, of saturated aqueous sodium bicarbonate, and then extracted twice with 50 mL of ethyl acetate. The organic layer was washed with 25 ml of saline, 25 mL of saturated aqueous sodium bicarbonate, and 25 mL of saline in this order, and then dewatered with anhydrous sodium sulfate. The drying agent was separated by filtration, and the filtrate was concentrated, and the residues were purified by silica gel column chromatography, thereby obtaining 2.81 g of a compound (CF) (Rf₁ in Formula (CF) is a PFPE chain represented by Formula (4-1), and in Rf₁, h representing the average degree of polymerization represents 4.5, and i representing the average degree of polymerization represents 4.5).

The structure of the obtained compound (CF) was identified by performing KH-NMR measurement and ¹⁹F-NMR measurement based on the following results.

¹H-NMR (acetone-D₆): δ [ppm]=1.65-1.85 (4H), 3.40-3.85 (26H), 3.85-4.10 (4H)

¹⁹F-NMR (acetone-D₆): δ [ppm]=−84.0 to −83.0 (18F), −86.4 (4F), −124.3 (4F), −130.0 to −129.0 (9F)

### Example 34

A compound represented by Formula (CO) was obtained by the following method.

2.92 g of a compound (CG) (Rf₂ in Formula (CG) is a PFPE chain represented by Formula (4-2), and in Rf₂, j representing the average degree of polymerization represents 4.5) was obtained by performing the same operation as in Example 33 except that a compound represented by Formula (5-26) was used instead of the compound represented by Formula (5-25).

The compound represented by Formula (5-26) was synthesized by the following method. First, a hydroxy group of 5-hexene-1-ol was protected using dihydropyran, and then the alkenyl group was oxidized with n-chloroperbenzoic acid. Thereafter, the obtained compound reacted with solketal, and the secondary hydroxy group of the generated compound was protected using chloromethyl methyl ether. The obtained compound was treated with an acid to selectively deprotect the THP group, and a primary hydroxy group reacted with p-toluenesulfonyl chloride, thereby obtaining a compound represented by Formula (5-26).

The structure of the obtained compound (CG) was identified by performing ¹H-NMR measurement and ¹⁹F-NMR measurement based on the following results.

¹H-NMR (acetone-D₆): δ [ppm]=1.65-1.85 (12H), 3.40-3.85 (26H), 3.85-4.10 (4H)

¹⁹F-NMR (acetone-Dr): δ [ppm]=−84.0 to −83.0 (18F), −86.4 (4F), −124.3 (4F), −130.0 to −129.0 (9F)

### Example 35

A compound represented by Formula (CH) was obtained by the following method.

A 100 mL eggplant flask was charged with 12 g of a compound represented by HOCH₂CF₂CF₂O(CF₂CF₂F₂O)CF₂CF₂CH₂OH (j representing the average degree of polymerization in the formula represents 4.5) (number-average molecular weight: 1,000, molecular weight distribution: 1.1), 1.81 g of the compound represented by Formula (5-27), and 20 mL of N,N-dimethylformamide in a nitrogen gas atmosphere, and the mixture was stirred at room temperature until the mixture was uniform. 5.51 g of cesium carbonate was added to the mixture, and the mixture was stirred at 50° C. for 16 hours to carry out a reaction.

The compound represented by Formula (5-27) was synthesized by protecting a 1,2-diol moiety of 1,2,6-hexanetriol with acetone and then reacting a hydroxy group at the 6-position with p-toluenesulfonyl chloride.

The reaction product obtained after the reaction was cooled to 25° C., transferred to a separatory funnel charged with 100 mL of water, and extracted three times with 100 mL of ethyl acetate. The organic layer was washed with water and dewatered with anhydrous sodium sulfate. The drying agent was separated by filtration, and the filtrate was concentrated, and the residues were purified by silica gel column chromatography, thereby obtaining 5.96 g of a compound represented by Formula (9) as an intermediate compound 1.

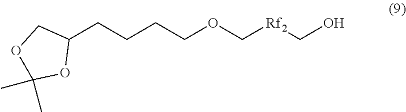

(Rf₂ in Formula (9) is a PFPE chain represented by Formula (4-2), in Rf₁ j representing the average degree of polymerization represents 4.5.)

Subsequently, a 100 mL eggplant flask was charged with 5.96 g of the compound represented by Formula (9) as an intermediate compound 1 obtained above, 2.76 g of the compound represented by Formula (5-26), and 20 mL of N,N-dimethylformamide in a nitrogen gas atmosphere, and the mixture was stirred at room temperature until the mixture is uniform. 3.65 g of cesium carbonate was added to the mixture, and the mixture was stirred at SOC for 16 hours to carry out a reaction.

The reaction solution obtained after the reaction was cooled to room temperature, 50 g of a 10% hydrogen chloride/methanol solution (hydrogen chloride-methanol reagent (5-10%), manufactured by Tokyo Chemical Industry Co., Ltd.) was added thereto, and the mixture was stirred at room temperature for 4 hours. Thereafter, the reaction solution was gradually transferred to a separatory funnel containing 100 mL of saturated aqueous sodium bicarbonate, and then extracted twice with 200 mL of ethyl acetate. The organic layer was washed with 100 mL of saline, 100 mL of saturated aqueous sodium bicarbonate, and 100 ml of saline in this order, and then dewatered with anhydrous sodium sulfate. The drying agent, was separated by filtration, and the filtrate was concentrated, and the residues were purified by silica gel column chromatography, thereby obtaining 2.41 g of a compound (CH) (Rf₂ in Formula (CH) is a PFPE chain represented by Formula (4-2), and in Rf₂, j representing the average degree of polymerization represents 4.5).

The structure of the obtained compound (CH) was identified by performing ¹H-NMR measurement and ¹⁹F-NMR measurement based on the following results.

¹H-NMR (acetone-1.6): δ [ppm]=1.65-1.85 (12H), 3.40-3.85 (20H), 3.85-4.10 (4H)

¹⁹F-NMR (acetone-D₆): δ [ppm]=−84.0 to −83.0 (18F), −86.4 (4F), −124.3 (4F), −130.0 to −129.0 (9F)

### Example 36

A compound represented by Formula (CI) was obtained by the following method.

3.13 g of a compound (CI) (Rf₂ in Formula (CI) is a PFPE chain represented by Formula (4-2), and in Rf₂, j representing the average degree of polymerization represents 4.5) was obtained by performing the same operation as in Example 26 except that a compound represented by Formula (5-8) was used instead of the compound represented by Formula (5-1).

The structure of the obtained compound (CI) was identified by performing ¹H-NMR measurement and ¹⁹F-NMR measurement based on the following results.

¹H-NMR (acetone-D₆): δ [ppm]=1.65-1.85 (8H), 3.40-3.85 (18H), 3.85-4.10 (4H)

¹⁹F-NMR (acetone-D₆): δ [ppm]=−84.0 to −83.0 (18F), −86.4 (4F), −124.3 (41F), −130.0 to −129.0 (9F)

### Example 37

A compound represented by Formula (CJ) was obtained by the following method.

3.52 g of a compound (C) (Rf₂ in Formula (CJ) is a PFPE chain represented by Formula (4-2), and in Rf₂, j representing the average degree of polymerization represents 4.5) was obtained by performing the same operation as in Example 26 except that a compound represented by Formula (5-10) was used instead of the compound represented by Formula (5-1).

The structure of the obtained compound (CJ) was identified by performing ¹H-NMR measurement and ¹⁹F-NMR measurement based on the following results.

¹H-NMR (acetone-D₆): δ [ppm]=1.65-1.85 (4H), 3.40-3.85 (34H), 3.85-4.10 (4H)

¹⁹F-NMR (acetone-D₆): δ [ppm]=−84.0 to −83.0 (18F), −86.4 (4F), −124.3 (4F), 130.0 to −129.0 (9F)

### Example 38

A compound represented by Formula (CK) was obtained by the following method.

3.61 g of a compound (CK) (Rf₂ in Formula (CK) is a PFPE chain represented by Formula (4-2), and in Rf₂, j representing the average degree of polymerization represents 4.5) was obtained by performing the same operation as in Example 26 except that a compound represented by Formula (5-15) was used instead of the compound represented by Formula (5-1).

The structure of the obtained compound (CK) was identified by performing ¹H-NMR measurement and ¹⁹F-NMR measurement based on the following results.

¹H-NMR (acetone-D₆): δ [ppm]=3.40-3.85 (26H), 3.85-4.10 (121H)

¹⁹F-NMR (acetone-Do: δ [ppm]=−84.0 to −83.0 (18F), −86.4 (4F), −124.3 (4F), −130.0 to −128.5 (17F)

The structures of R¹, R², and R³ in a case where the compounds (AA) to (AS), (BA) to (BH), and (CA) to (CK) of Examples 1 to 38 obtained as described above were each applied to Formula (1) are listed in Tables 1 and 2.

TABLE 1

| Compound | R¹ | | R² | R³ | |
|---|---|---|---|---|---|
| (AA) | (2-1) | a = 1/b = 1 | (4-1) | (2-1) | a = 1/b = 1 |
| (AB) | (2-1) | a = 1/b = 3 | (4-1) | (2-1) | a = 1/b = 3 |
| (AC) | (2-1) | a = 1/b = 5 | (4-1) | (2-1) | a = 1/b = 5 |
| (AD) | (2-1) | a = 2/b = 1 | (4-1) | (2-1) | a = 2/b = 1 |
| (AE) | (2-2) | c = 1 | (4-1) | (2-2) | c = 1 |
| (AF) | (2-2) | c = 3 | (4-1) | (2-2) | c = 3 |
| (AG) | (2-2) | c = 5 | (4-1) | (2-2) | c = 5 |
| (AH) | (2-3) | d = 2 | (4-1) | (2-3) | d = 2 |
| (AI) | (2-3) | d = 4 | (4-1) | (2-3) | d = 4 |
| (AJ) | (2-4) | e = 1/R [symbol] = hydrogen atom/ | (4-1) | (2-4) | e = 1/R [symbol] = hydrogen atom/ |
| | | R [symbol] = hydrogen atom | | | R [symbol] = hydrogen atom |
| (AK) | (2-4) | e = 1/R [symbol] = methyl group/ | (4-1) | (2-4) | e = 1/R [symbol] = methyl group/ |
| | | R [symbol] = methyl group | | | R [symbol] = methyl group |
| (AL) | (2-4) | e = 2/R [symbol] = hydrogen atom/ | (4-1) | (2-4) | e = 2/R [symbol] = hydrogen atom/ |
| | | R [symbol] = hydrogen atom | | | R [symbol] = hydrogen atom |
| (AM) | (2-4) | e = 2/R [symbol] = hydrogen atom/ | (4-1) | (2-4) | e = 2/R [symbol] = hydrogen atom/ |
| | | R [symbol] = methyl group | | | R [symbol] = methyl group |
| (AN) | (2-4) | e = 6/R [symbol] = hydrogen atom/ | (4-1) | (2-4) | e = 6/R [symbol] = hydrogen atom/ |
| | | R [symbol] = hydrogen atom | | | R [symbol] = hydrogen atom |
| (AO) | (2-5) | f = 2 | (4-1) | (2-5) | f = 2 |
| (AP) | (2-5) | f = 6 | (4-1) | (2-5) | f = 6 |
| (AQ) | (2-6) | g = 2 | (4-1) | (2-6) | g = 2 |
| (AR) | (2-6) | g = 5 | (4-1) | (2-6) | g = 5 |
| (AS) | (2- [symbol] ) | a = 1/b = 1 | (4-1) | (2-2) | c = 1 |

[symbol]

indicates data missing or illegible when filed

TABLE 2

| Compound | R¹ | | R² | R³ | |
|---|---|---|---|---|---|
| (BA) | (2-1) | a = 1/b = 1 | (4-1) | (3-1) | p = 0/q = 0/r = 1/ |
| | | | | | D = hydroxy group |
| (BB) | (2-1) | a = 1/b = 1 | (4-1) | (3-1) | p = 1/q = 1/r = 1/ |
| | | | | | D = vinyl group |
| (BC) | (2-1) | a = 1/b = 1 | (4-1) | (3-1) | p = 0/q = 0/r = 1/ |
| | | | | | D = acetamid [symbol] group |
| (BD) | (2-1) | a = 1/b = 1 | (4-1) | (3-1) | p = 0/q = 0/r = 2/ |
| | | | | | D = cyano group |
| (BE) | (2-1) | a = 1/b = 1 | (4-1) | (3-2) | [symbol] = 0/ [symbol] = 1 |
| (BF) | (2-1) | a = 1/b = 1 | (4-1) | (3) | l = 2/m = 1/n = 1/ |
| | | | | | B = hydrogen atom |
| (BG) | (2-1) | a = 1/b = 1 | (4-2) | (2-1) | a = 1/b = 1 |
| (BH) | (2-1) | a = 1/b = 1 | (4-3) | (2-1) | a = 1/b = 1 |
| (CA) | (2-1) | a = 1/b = 1 | (4-1) | (2-1) | a = 1/b = 1 |
| (CB) | (2-2) | c = 1 | (4-1) | (2-2) | c = 1 |
| (CC) | (2-2) | c = 1 | (4-2) | (2-2) | c = 1 |
| (CD) | (2-2) | c = 1 | (4-3) | (2-2) | c = 1 |
| (CE) | (2-4) | e = 1/ | (4-1) | (2-4) | e = 1/ |
| | | R [symbol] = hydrogen atom/ | | | R [symbol] = hydrogen atom/ |
| | | R [symbol] = methyl group | | | R [symbol] = methyl group |
| (CF) | (2-7) | g2 = 1 | (4-2) | (2-7) | g2 = 1 |
| (CG) | (2-7) | g2 = 3 | (4-2) | (2-7) | g2 = 3 |
| (CH) | (2-6) | g = 3 | (4-2) | (2-7) | g2 = 3 |
| (CI) | (2-3) | d = 2 | (4-2) | (2-3) | d = 2 |
| (CJ) | (2-4) | e = 1/ | (4-2) | (2-4) | e = 1/ |
| | | R [symbol] = hydrogen atom/ | | | R [symbol] = hydrogen atom/ |
| | | R [symbol] = hydrogen atom | | | R [symbol] = hydrogen atom |
| (CK) | (2-5) | f = 2 | (4-2) | (2-5) | f = 2 |

[symbol]

indicates data missing or illegible when filed

### Comparative Example 1

A compound represented by Formula (ZA) was synthesized by the method described in Patent Document 1.

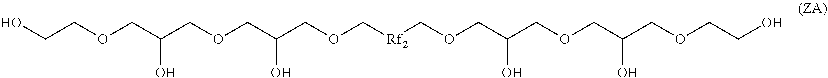

(Rf₂ in Formula (ZA) is a PFPE chain represented by Formula (4-2). In Rf₂, j representing the average degree of polymerization represents 4.5.)

### Comparative Example 2

A compound represented by Formula (ZB) was synthesized by the method described in Patent Document 2.

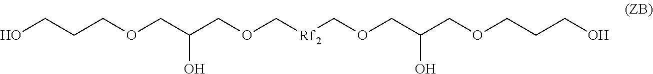

(Rf₁ in Formula (ZB) is a PFPE chain represented by Formula (4-2). In Rf₂, j representing the average degree of polymerization represents 4.5.)

### Comparative Example 3

A compound represented by Formula (ZC) was synthesized by the method described in Patent Document 3,

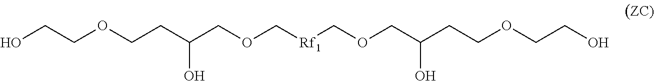

(Rf₁ in Formula (ZC) is a PFPE chain represented by Formula (4-1). In Rf₁, h representing the average degree of polymerization represents 4.5, and i representing the average degree of polymerization represents 4.5.)

### Comparative Example 4

A compound represented by the following formula (Z)) was synthesized by the following method.

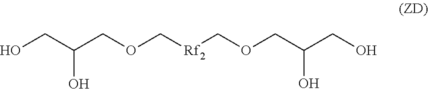

(Rf₂ in Formula (ZD) is a PFPE chain represented by Formula (4-2). In Rf₂, j representing the average degree of polymerization represents 4.5.)

2.81 g of a compound (ZD) was obtained by performing the same operation as in Example 1 except that a compound represented by Formula (5-23) was used instead of the compound represented by Formula (5-1) and a compound represented by HOCH₂CF₂CF₂O(CF₂CF₂CF₂O)ⱼCF₂CF₂CH₂OH (j representing the average degree of polymerization in the formula represents 4.5) (number-average molecular weight: 1,000, molecular weight distribution: 1.1) was used instead of the compound represented by HOCH₂CF₂O(CF₂CF₂O)ₕ(CF₂O)ᵢCF₂CH₂OH.

The structure of the obtained compound (ZI)) was identified by performing ¹H-NMR measurement and ¹⁹F-NMR measurement based on the following results.

¹H-NMR (acetone-D₆): δ [ppm]=3.40-3.85 (14H), 3.85-4.10 (4H)

¹⁹F-NMR (acetone-D₆): δ [ppm]=−84.0 to −83.0 (18F), −86.4 (4F), −124.3 (4F), −130.0 to −129.0 (9F)

### Comparative Example 5

A compound represented by Formula (ZE) was synthesized by the method described in Patent Document 4.

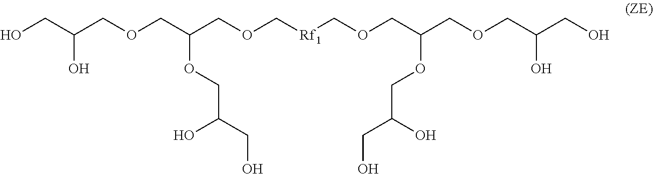

(Rf₁ in Formula (ZE) is a PFPE chain represented by Formula (4-1). In Rf₁, h representing the average degree of polymerization represents 4.5, and i representing the average degree of polymerization represents 4.5.)

The number-average molecular weights (Mn) of the compounds of Examples 1 to 38 and Comparative Examples 1 to 5, which wee obtained as described above, were measured by the above-described method. The results are listed in Tables 3 and 4.

Next, a lubricating layer forming solution was prepared using the compounds obtained in Examples 1 to 38 and Comparative Examples 1 to 5 by the following method. Next, a lubricating layer of a magnetic recording medium was formed by the following method using the obtained lubricating layer forming solution, thereby obtaining magnetic recording media of Examples 1 to 38 and Comparative Examples 1 to 5.

### “Solution for Forming Lubricating Layer”

The compounds obtained in Examples 1 to 38 and Comparative Examples 1 to 5 were each dissolved in VERTREL (registered trademark) XF (trade name, manufactured by DuPont Mitsui Fluorochemicals Co., Ltd.), which is a fluorine-based solvent, and diluted with VERTREL XF such that the film thickness in a case where the solution was applied onto the protective layer reached 9.0 Å to 9.5 Å, thereby preparing a lubricating layer forming solution.

### “Magnetic Recording Medium”

A magnetic recording medium in which an adhesive layer, a soft magnetic layer, a first underlayer, a second underlayer, a magnetic layer, and a protective layer were sequentially provided on a substrate having a diameter of 65 mm was prepared. The protective layer was formed of carbon.

The protective layer of the magnetic recording medium on which each layer up to the protective layer was formed was coated with each lubricating layer forming solution of Examples 1 to 38 and Comparative Examples 1 to 5 by a dipping method. Further, the dipping method was carried out under the conditions of an immersion speed of 10 mm/sec, an immersion time of 30 sec, and a pulling-up speed of 1.2 mm/sec.

Thereafter, the magnetic recording medium coated with the lubricating layer forming solution was placed in a constant temperature tank, and a thermal treatment was performed at 120° C. for 10 minutes to remove the solvent in the lubricating layer forming solution and to improve the adhesion between the protective layer and the lubricating layer so that a lubricating layer was formed on the protective layer, thereby obtaining a magnetic recording medium.

### [Measurement of Film Thickness]

The film thicknesses of the lubricating layers of the magnetic recording media of Examples 1 to 38 and Comparative Examples 1 to 5 obtained above were measured using a Fourier transform infrared spectrophotometer (FT-IR, trade name: Nicolet iS50, manufactured by Thermo Fisher Scientific). The results are listed in Tables 3 and 4.

Next, the magnetic recording media of Examples 1 to 38 and Comparative Examples 1 to 5 were subjected to the corrosion resistance test and the spin-off characteristic test described below. The results are listed in Tables 3 and 4.

### [Corrosion Resistance Test]

The magnetic recording medium was exposed to an environment of a temperature of 85° C., and a relative humidity of 90% for 48 hours. Thereafter, the number of the corrosion spots having a diameter of 5 μm or greater generated on the surface of the magnetic recording medium was counted using an optical surface analyzer (Candela 7140, manufactured by KLA-Tencor Corporation), and evaluated according to the following evaluation criteria.

### “Evaluation Criteria of Corrosion Resistance”

A+: The number of corrosion spots was less than 10.
A: The number of corrosion spots was 100 or more and less than 300.
R: The number of corrosion spots was 300 or more and less than 500.
C: The number of corrosion spots was 500 or more and less than 1.000.
D: The number of corrosion spots was 1,000 or more.

### [Spin-Off Characteristic Test]

The magnetic recording medium was mounted on a spin stand, and spin stand rotated in an environment of 80° C. at a rotation speed of 10000 rpm for 72 hours. Before and after this operation, the film thickness of the lubricating layer at a position with a radius of 20 mm from the center of the magnetic recording medium was measured using FT-IR, and the rate of a decrease in the film thickness of the lubricating layer before and after the test was calculated. The spin-off characteristics were evaluated according to the evaluation criteria described below using the calculated rate of a decrease in the film thickness.

### “Evaluation Criteria of Spin-Off Characteristics”

A+: The rate of a decrease in the film thickness was less than 2%.
A: The rate of a decrease in the film thickness was 2% or greater and less than 3%.
B: The rate of a decrease in the film thickness was 3% or greater and less than 5%.
C: The rate of a decrease in the film thickness was 5% or greater and less than 10%.
D: The rate of a decrease in the film thickness was 10% or greater.

### [Comprehensive Evaluation]

Based on the results of the corrosion resistance test and the spin-off characteristic test, the comprehensive evaluation was performed according to the following criteria.

### “Comprehensive Evaluation Criteria”

A: Both the evaluation of the corrosion resistance test and the evaluation of the spin-off characteristic test were A+ or A.
B: One of the evaluation of the corrosion resistance test or the evaluation of the spin-off characteristic test was B, and the other was A+, A, or B.
C: One of the evaluation of the corrosion resistance test or the evaluation of the spin-off characteristic test was C, and the other was A+, A, b, or C.
D: At least one of the evaluation of the corrosion resistance test or the evaluation of the spin-off characteristic test was D.

TABLE 3

| | | | | Corrosion | Spin-off | |
|---|---|---|---|---|---|---|
| | | Molecular | thickness | resistance | characteristic | Comprehensive |
| | Compound | weight | (Å) | test | test | evaluation |
| Example 1 | (AA) | 1315 | 9.0 | A | A+ | A |
| Example 2 | (AB) | 1365 | 9.4 | A | A+ | A |
| Example 3 | (AC) | 1418 | 9.2 | A | A | A |
| Example 4 | (AD) | 1341 | 9.2 | A | A+ | A |
| Example 5 | (AE) | 1322 | 9.5 | A | A+ | A |
| Example 6 | (AF) | 1363 | 9.3 | A | A+ | A |
| Example 7 | (AG) | 1416 | 9.1 | A | A | A |
| Example 8 | (AH) | 1284 | 9.0 | A+ | A | A |
| Example 9 | (AI) | 1301 | 9.4 | A+ | B | B |
| Example 10 | (AJ) | 1406 | 9.5 | A | A+ | A |
| Example 11 | (AK) | 1467 | 9.5 | A | A+ | A |
| Example 12 | (AL) | 1436 | 9.5 | A | A+ | A |
| Example 13 | (AM) | 1500 | 9.3 | A | A+ | A |
| Example 14 | (AN) | 1552 | 9.4 | A | A | A |
| Example 15 | (AO) | 1473 | 9.3 | A+ | A | A |
| Example 16 | (AP) | 1680 | 9.2 | A+ | A | A |
| Example 17 | (AQ) | 1202 | 9.5 | A+ | A | A |
| Example 18 | (AR) | 1289 | 9.3 | A+ | B | B |
| Example 19 | (AS) | 1320 | 9.2 | A | A+ | A |

TABLE 4

| | | | | Corrosion | Spin-off | |
|---|---|---|---|---|---|---|
| | | Molecular | thickness | resistance | characteristic | Comprehensive |
| | Compound | weight | (Å) | test | test | evaluation |
| Example 20 | (BA) | 1279 | 9.4 | A | A | A |
| Example 21 | (BB) | 1372 | 9.0 | A | A | A |
| Example 22 | (BC) | 1328 | 9.4 | A | A | A |
| Example 23 | (BD) | 1304 | 9.5 | A | A | A |
| Example 24 | (BE) | 1241 | 9,0 | A | A | A |
| Example 25 | (BF) | 1302 | 9.5 | A | A | A |
| Example 26 | (BG) | 1323 | 9.0 | A | A+ | A |
| Example 27 | (BH) | 1319 | 9.3 | A | A+ | A |
| Example 28 | (CA) | 1324 | 9.3 | A | A+ | A |
| Example 29 | (CB) | 1321 | 9.4 | A | A+ | A |
| Example 30 | (CC) | 1313 | 9.1 | A | A+ | A |
| Example 31 | (CD) | 1310 | 9.0 | A | A+ | A |
| Example 32 | (CE) | 1328 | 9.2 | A | A+ | A |
| Example 33 | (CF) | 1322 | 9.3 | A | A+ | A |
| Example 34 | (CG) | 1385 | 9.1 | A | A+ | A |
| Example 35 | (CH) | 1307 | 9.4 | A+ | A | A |
| Example 36 | (CI) | 1261 | 9.0 | A+ | A | A |
| Example 37 | (CJ) | 1399 | 9.2 | A | A+ | A |
| Example 38 | (CK) | 1478 | 9.3 | A+ | A | A |
| Comparative | (ZA) | 1380 | 9.0 | D | C | D |
| Example 1 | | | | | | |
| Comparative | (ZB) | 1267 | 9.0 | B | D | D |
| Example 2 | | | | | | |
| Comparative | (ZC) | 1274 | 9.1 | B | D | D |
| Example 3 | | | | | | |
| Comparative | (ZD) | 1132 | 9.4 | C | C | C |
| Example 4 | | | | | | |
| Comparative | (ZE) | 1446 | 9.5 | D | C | D |
| Example 5 | | | | | | |

As listed in Tables 3 and 4 in all of the magnetic recording media of Examples 1 to 38 in which at least one of R¹ or R³ disposed at the end of the perfluoropolyether chain represents an end group represented by Formula (2) and the fluorine-containing ether compounds (AA) to (AS), (BA) to (BH), and (CA) to (CK) satisfying Formula (1) were used, the evaluations of the corrosion resistance test and the spin-off characteristic test were A+, A, or B, and the comprehensive evaluation was A or B. As shown in the results, it was confirmed that the lubricating layers of the magnetic recording media of Examples 1 to 38 had satisfactory corrosion resistance and was capable of suppressing spin-off.

In Examples 8, 9, 15 to 18, 35, 36, and 38 in which the compounds (AH), (AI), (AO) to (AR), (CH), (CI), and (CK) in which R¹ and R³ represent an end group represented by any of Formulae (2-3), (2-5), (2-6), and (2-7) were used, the evaluation of the corrosion resistance test was A+, which showed a satisfactory result.

In Examples 1, 2, 4 to 6, 10 to 13, 19, and 26 to 34, and 37 in which compounds (AA), (AB), (AD). (BG). (BH), and (CA) in which R¹ and R³ represent an end group represented by Formula (2-1) and the sum of a and b was 4 or less, compounds (AE), (AF), (CB), (CC), and (CD) in which R¹ and R³ represent an end group represented by Formula (2-2) and c represents 3 or less, compounds (AJ) to (AM), (CE), and (CJ) in which R¹ and R³ represent an end group represented by Formula (2-4) and e represents 2 or less, a compound (AS) in which one of R¹ and R³ represents an end group represented by Formula (2-1), the sum of a and b was 4 or less, the other represents an end group represented by Formula (2-2), and c represents 3 or less, and compounds (CF) and (CG) in which R¹ and R² represent an end group represented by Formula (2-7) and g2 represents 3 or less were used, the evaluation of the spin-off characteristic test was A+, which showed a satisfactory result.

On the other hand, as listed in Tables 3 and 4, in Comparative Examples 1 to 5 in which the compounds (ZA) to (ZB) were used, all the evaluations of the corrosion resistance test and the spin-off characteristic test were any of B, C, or D, and the comprehensive evaluation was any of C or D.

In the compound (ZA) used in Comparative Example 1, the end group did not have a 1,2-diol structure and a carbon atom not bonded to any of the polar group and the ether oxygen atom.

The end group of the compound (ZA) does not have a 1,2-diol structure, and all hydroxy groups thereof greatly contribute to an increase in the polarity of the entire molecule. In addition, since the end group of the compound (ZA) does not have a carbon atom bonded to neither the polar group nor the ether oxygen atom, the hydrophobicity of the molecule is not sufficiently obtained. As shown in the results, in Comparative Example 1, it was considered that the fluorine-containing ether compound easily took in water, which causes corrosion, and the result of the corrosion resistance test was D.

In the compound (ZA), since the distance between the hydroxy groups was larger than the distance between the hydroxy groups of the 1,2-diol structure, all the hydroxy groups were likely to interact with the protective layer. As a result, it was considered that since the interaction between the hydroxy groups in the molecules was small, the lubricant was likely to be scattered with the rotation of the magnetic recording medium, and the evaluation of the spin-off characteristic test was C.

In the compound (ZB) used in Comparative Example 2 and the compound (ZC) used in Comparative Example 3, the end group had a carbon atom that was not bonded to either the polar group or the ether oxygen atom, but did not have a 1,2-diol structure.

In the compound (ZB) and the compound (ZC), the end group had a carbon atom not bonded to any of the polar group and the ether oxygen atom, and thus the hydrophobicity of the molecules was increased. In addition, the total number of hydroxy groups in the compound (ZB) and the compound (ZC) is 4. Therefore, the total number of hydroxy groups in the compound (ZS) and the compound (ZC) is smaller than the total number of hydroxy groups, which is 6, in the compound (ZA) used in Comparative Example 1. Therefore, in the compound (ZB) and the compound (ZC), since the fluorine-containing ether compound is unlikely to take in water, which caused corrosion, the results of the corrosion resistance test of Comparative Example 2 and Comparative Example 3 were more satisfactory than those of Comparative Example 1.

In the compound (ZB) and the compound (ZC), since the distance between the hydroxy groups was larger than the distance between the hydroxy groups of the 1,2-diol structure, all the hydroxy groups easily interacted with the protective layer. In addition, the total number of hydroxy groups in the compounds (ZB) and (ZC) was 4, which was relatively small, and thus almost no hydroxy groups that could contribute to the intermolecular interaction were present. As a result, it was considered that since the intermolecular interaction between the fluorine-containing ether compounds was small, and the lubricant was likely to be scattered with the rotation of the magnetic recording medium, the evaluation of the spin-off characteristic test of Comparative Example 2 and Comparative Example 3 was D.

The compound (ZD) used in Comparative Example 4 and the compound (ZE) used in Comparative Example 5 had a 1,2-diol structure in the end group, but did not have a carbon atom not bonded to either the polar group or the ether oxygen atom.

Since the compound (ZD) and the compound (ZE) did not have a carbon atom not bonded to either the polar group or the ether oxygen atom in the end group, the hydrophobicity of the molecules was decreased. As a result, it was considered that since the fluorine-containing ether compound easily took in water, which caused corrosion, the result of the corrosion resistance test of Comparative Example 4 was C, and the result of the corrosion resistance test of Comparative Example 5 was D. The reason why the result of the corrosion resistance test of Comparative Example 4 was C and the result thereof of Comparative Example 5 was D was considered to be that the total number of hydroxy groups in the compound (ZD) was 4 and the total number of hydroxy groups in the compound (ZE) was 8, and thus the total number of hydroxy groups was larger in Comparative Example 5, which made it easier to take in water.

Since the compound (ZD) and the compound (ZE) did not have a carbon atom not bonded to either the polar group or the ether oxygen atom at the end, the moderate rigidity was unlikely to be imparted to the molecule. Therefore, two hydroxy groups constituting the 1,2-diol structure were likely to interact with each other in the molecule. As a result, it was considered that since the hydroxy groups in the fluorine-containing ether compound were less likely to interact with each other between molecules, and the lubricant was likely to be scattered with the rotation of the magnetic recording medium, the evaluation of the spin-off characteristic test of Comparative Example 4 and Comparative Example 5 was C.

### INDUSTRIAL APPLICABILITY

A lubricating layer having satisfactory corrosion resistance even in a case where the thickness thereof is small and capable of suppressing spin-off can be formed by using the lubricant for a magnetic recording medium, which contain, the fluorine-containing ether compound of the present invention.

### REFERENCE SIGNS LIST

10: magnetic recording medium
11: substrate
12: adhesive layer
13: soft magnetic layer
14: first underlayer
15: second underlayer
16: magnetic layer
17: protective layer
18: lubricating layer

1. A fluorine-containing ether compound which is represented by Formula (1), 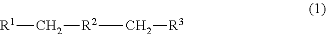
(in Formula (1), R² represents a perfluoropolyether chain; R¹ and R³ represent an end group having one to four polar groups and 1 to 50 carbon atoms; R¹ and R³ may be the same as or different from each other; and at least one of R¹ or R³ represents an end group represented by Formula (2)), 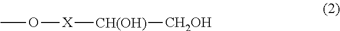
(in Formula (2), X represents a divalent organic group having 2 to 30 carbon atoms, which may have at least one of one or two polar groups or one to three ether oxygen atoms; and X has at least one carbon atom which is bonded to neither of the polar groups nor the ether oxygen atoms).

2. The fluorine-containing ether compound according to claim 1,
wherein the end group represented by Formula (2) is a group represented by any of Formulae (2-1) to (2-7), 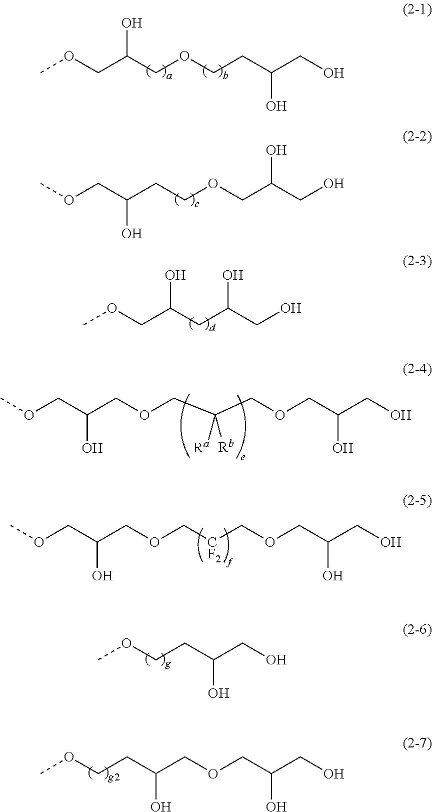
(in Formula (2-1), a represents an integer of 1 to 8; and b represents an integer of 1 to 7),
(in Formula (2-2), c represents an integer of 1 to 7),
(in Formula (2-3), d represents an integer of 1 to 6),
(in Formula (2-4), e represents an integer of 1 to 6; and e pieces of R^{a}'s and R^{b}'s each independently represent a hydrogen atom or a methyl group),
(in Formula (2-5), f represents an integer of 1 to 6),
(in Formula (2-6), g represents an integer of 1 to 6),
(in Formula (2-7), g2 represents an integer of 1 to 6).

3. The fluorine-containing ether compound according to claim 1,
wherein R¹ and R³ in Formula (1) each independently represent an end group represented by Formula (2).

4. The fluorine-containing ether compound according to claim 1,
wherein R¹ and R³ in Formula (1) are the same as each other.

5. The fluorine-containing ether compound according to claim 1,
wherein one of R¹ or R³ in Formula (1) represents an end group represented by Formula (2) and the other represents an end group represented by Formula (3) not corresponding to Formula (2), 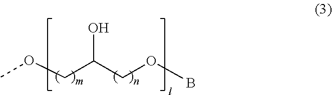
(in Formula (3), 1 represents an integer of 1 to 3; l pieces of m's each independently represent an integer of 1 to 6; l pieces of n's each independently represent an integer of 1 to 6; in one repeating unit, at least one of m or n represents 1; and B represents an alkyl group which may have only one polar group, an organic group having one or more carbon-carbon unsaturated bonds, or a hydrogen atom).

6. The fluorine-containing ether compound according to claim 1,
wherein one of R¹ or R³ in Formula (1) represents an end group represented by Formula (2) and the other represents an end group represented by any of Formulae (3-1) to (3-3), 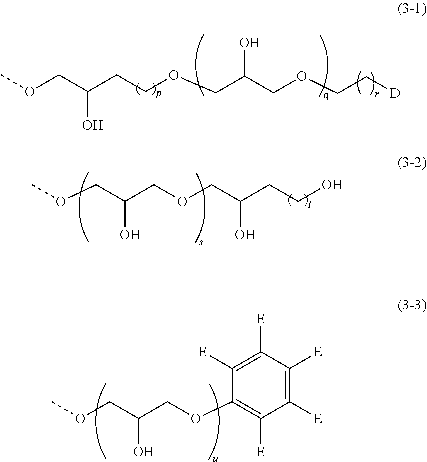
(in Formula (3-1), p represents an integer of 0 to 3, q represents an integer of 0 to 2, r represents an integer of 0 to 5, a total value of p and r is in a range of 1 to 5, and D represents a polar group, a vinyl group, an ethynyl group, or an aryl group which may have a substituent),
(in Formula (3-2), s represents an integer of 0 to 2; and t represents an integer of 1 to 5),
(in Formula (3-3), u represents an integer of 1 to 3; and five E's each independently represent a polar group, an alkoxy group having 1 to 8 carbon atoms, a halogeno group, or a hydrogen atom, where a number of polar groups in the five E's is 1 in a case where the five E's include polar groups).

7. The fluorine-containing ether compound according to claim 1,
wherein a total number of polar groups included in R¹ and R³ in Formula (1) is in a range of 4 to 6.

8. The fluorine-containing ether compound according to claim 1,
wherein R² in Formula (1) represents a perfluoropolyether chain represented by Formula (4), 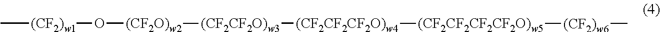
(in Formula (4), w2, w3, w4, and w5 represent an average degree of polymerization, and each independently represent 0 to 20, where all of w2, w3, w4, and w5 do not represent 0 at the same time; w1 and w6 represent an average value representing the number of CF₂'s, and each independently represent 1 to 3; and an arrangement order of (CF₂O), (CF₂CF₂O), (CF₂CF₂CF₂O), and (CF₂CF₂CF₂CF₂O), which are the repeating units in Formula (4), is not particularly limited).

9. The fluorine-containing ether compound according to claim 1,
wherein R² in Formula (1) represents any one selected from the group consisting of perfluoropolyether chains represented by any of Formulae (4-1) to (4-4), 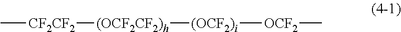
(in Formula (4-1), h and i represent an average degree of polymerization, where h represents 1 to 20 and i represents 0 to 20), 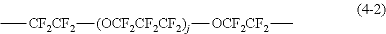
(in Formula (4-2), j represents an average degree of polymerization and represents 1 to 15), 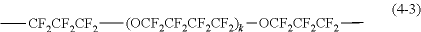
(in Formula (4-3), k represents an average degree of polymerization and represents 1 to 10), 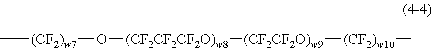
(in Formula (4-4), w8 and w9 represent an average degree of polymerization, and each independently represents 1 to 20; and w7 and w10 represent an average value representing the number of CF₂'s, and each independently represent 1 or 2).

10. The fluorine-containing ether compound according to claim 1,
wherein the fluorine-containing ether compound has a number-average molecular weight of 500 to 10000.

11. A lubricant for a magnetic recording medium, comprising:
the fluorine-containing ether compound according to claim 1.

12. A magnetic recording medium, which is provided with at least a magnetic layer, a protective layer, and a lubricating layer in this order on a substrate,
wherein the lubricating layer contains the fluorine-containing ether compound according to claim 1.

13. The magnetic recording medium according to claim 12,
wherein the lubricating layer has an average film thickness of 0.5 nm to 2.0 nm.
